(19) United States
(12) Patent Application Publication
(10) Pub. No.: US 2014/0315933 A1
(43) Pub. Date: Oct. 23, 2014

(54) CHEMICAL COMPOUNDS

(71) Applicant: Pfizer Limited, Sandwich (GB)

(72) Inventors: Robert McKenzie Owen, Great Abington (GB); Robert Ian Storer, Great Abington (GB)

(73) Assignee: PFIZER LIMITED, Sandwich (GB)

(21) Appl. No.: 14/255,110

(22) Filed: Apr. 17, 2014

Related U.S. Application Data
(60) Provisional application No. 61/930,025, filed on Jan. 22, 2014.
(60) Provisional application No. 61/813,796, filed on Apr. 19, 2013.

(51) Int. Cl. C07D 417/12; A61K 31/426; C07D 285/08; A61K 31/433; A61K 31/506; A61K 31/415; C07D 261/16; A61K 31/42; A61K 31/4439; C07D 285/135; C07D 277/52; C07D 231/42

(52) U.S. Cl. CPC C07D 417/12; C07D 277/52; A61K 31/426; C07D 285/08; A61K 31/433; C07D 231/42; A61K 31/415; C07D 261/16; A61K 31/42; A61K 31/4439; C07D 285/135; A61K 31/506
USPC 514/269; 548/197; 514/370; 548/128; 514/361; 548/371.7; 514/407; 548/246; 514/380; 546/270.7; 514/342; 548/138; 514/363; 546/268.7; 544/319

(57) ABSTRACT

The present invention relates to new sulfonamide URAT-1 inhibitor compounds of formula (I) or pharmaceutically acceptable salts thereof:

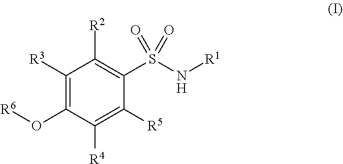

to compositions containing them, to processes for their preparation and to intermediates used in such processes and to methods of treatment; wherein R¹, R², R³, R⁴, R⁵ and R⁶ are as defined in the description.

This application claims the benefit under 35 U.S.C. §119(e) of U.S. Provisional Patent Application No. 61/930,025, filed on Jan. 22, 2014, and U.S. Provisional Patent Application No. 61/813,796, filed on Apr. 19, 2013, the disclosures of which are hereby incorporated by reference in their entirety.

### FIELD OF THE INVENTION

The invention relates to sulfonamide derivatives, to their use in medicine, to compositions containing them, to processes for their preparation and to intermediates used in such processes.

Uric acid is the final product of purine metabolism in humans. In humans, unlike many other animals, uric acid is not further broken down, but is predominantly (70%) excreted into the urine with the remaining 30% excreted in faeces. Hyperuricemia is defined as an excessive production or decreased excretion of uric acid and can occur as an overproduction or under excretion of serum uric acid (sUA), or a combination of the both. Renal under excretion of uric acid is the primary cause of hyperuricemia in about 90% of cases, while overproduction is the cause in less than 10%. Increased sUA concentration above 6.8 mg/dL results in crystallisation of uric acid in the form of salts, such as monosodium urate, and to precipitation of these crystals in joints, on tendons and in the surrounding tissues. These crystals (known as tophi) trigger a local immune-mediated inflammatory reaction, leading to gout. The risk of gout increases with increased sUA levels.

Gout is a painful condition that can present in a number of ways, although the most usual is a recurrent attack of acute inflammatory arthritis (a red, tender, hot, swollen joint) often occurring in big toes, heels, knees, wrists and fingers.

Gout is treated by agents to both decrease the cause and effects of uric acid crystal inflammation and pain.

The pain associated with gout is commonly treated with pain and anti-inflammatory drugs such as nonsteroidal anti-inflammatory drugs (NSAIDs), colchicine and steroids. Agents that decrease sUA levels may be used to treat the cause of gout. These include agents that: inhibit the enzymes that result in uric acid production, such as xanthine oxidase inhibitors (e.g. allopurinol, febuxostat or tisopurine), or purine nucleoside phosphorylase (PNP) inhibitors (e.g. ulodesine); metabolise uric acid, such as urate oxidases—also known as uricases (e.g. pegloticase); or increase the excretion of uric acid in the urine (uricosurics), Uricosurics include agents that inhibit the transporters responsible for renal reabsorption of uric acid back into the blood, such as benziodarone, isobromindione, probenecid and sulphinpyrazone, and URAT-1 inhibitors (e.g. benzbromarone).

URAT-1 is also known as solute carrier family 22 (organic anion/cation transporter), member 12, and is encoded by the gene SLC22A12. Human genetic analysis has demonstrated that polymorphisms in the SLC22A12 gene are directly associated with changes in serum uric acid. Inhibitors of uric acid transport, such as URAT-1, are therefore effective in the treatment of gout.

There is a continuing need to provide new treatments for gout that are more effective and/or are better tolerated.

Certain URAT-1 inhibitors for the treatment of gout are known. WO2011/159840 discloses phenylthioacetate URAT-1 inhibitors. Additionally, WO2008/118758, WO2009/012242, WO2010/079443, WO2012/004706, WO2012/004714 and WO2012/004743 disclose sulphonamides.

There is, however, an ongoing need to provide new URAT-1 inhibitors that are good drug candidates.

Furthermore, preferred compounds should have one or more of the following properties: be well absorbed from the gastrointestinal tract; be metabolically stable; have a good metabolic profile, in particular with respect to the toxicity or allergenicity of any metabolites formed; or possess favourable pharmacokinetic properties whilst still retaining their activity profile as URAT-1 inhibitors. They should be non-toxic and demonstrate few side-effects. Ideal drug candidates should exist in a physical form that is stable, non-hygroscopic and easily formulated.

### SUMMARY OF THE INVENTION

We have now found new sulphonamide URAT-1 inhibitors.

According to a first aspect of the invention there is provided a compound of formula (I)

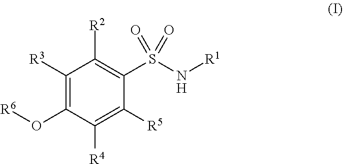

or a pharmaceutically acceptable salt thereof, wherein:

R¹ is a ‘C-linked’ 5-membered heteroaryl containing one, two or three heteroatoms selected from: (a) one to three nitrogen atoms, (b) one or two nitrogen atoms and one sulphur atom and (c) one or two nitrogen atoms and one oxygen atom, wherein said heteroaryl is optionally substituted on a ring carbon atom by, valency permitting, one, two or three X¹;

each X¹ is independently selected from: F; Cl; CN; (C₁-C₄)alkyl optionally substituted by one, two or three F; and (C₁-C₄)alkyloxy optionally substituted by one two or three F;

R², R³ and R⁵ are independently selected from: H; halogen; CN; (C₁-C₄)alkyl optionally substituted by one, two or three F; and (C₁-C₄)alkyloxy optionally substituted by one, two or three F;

R⁴ is selected from: halogen; CN; (C₁-C₄)alkyl optionally substituted by one, two or three F; and (C₁-C₄)alkyloxy optionally substituted by one, two or three F;

R⁶ is phenyl substituted by one, two or three X²; or a ‘C-linked’ 6-membered heteroaryl containing one or two nitrogen atoms wherein said heteroaryl is optionally substituted by one, two or three X²;

each X² is independently selected from: F, Cl; CN; —S(C₁-C₄)alkyl; —NR⁷R⁸; (C₁-C₆)alkyloxy optionally substituted by one, two or three F; (C₃-C₆)cycloalkyloxy; (C₁-C₆)alkyl optionally substituted by one, two or three F; and (C₁-C₆)alkyl substituted by OH; and

each R⁷ and R⁸ is independently H or (C₁-C₄)alkyl or, together with the nitrogen atom to which they are attached, form a saturated 4- to 6-membered nitrogen containing monocycle.

Described below are a number of embodiments (E) of this first aspect of the invention, where for convenience E1 is identical thereto.
E1 A compound of formula (I) as defined above or a pharmaceutically acceptable salt thereof.
E2 A compound according to E1 wherein R¹ is a ‘C-linked’ 5-membered heteroaryl containing one or two nitrogen atoms and one sulphur atom, wherein said heteroaryl is optionally substituted by one or two X¹.
E3 A compound according to E2 wherein R¹ is a ‘C-linked’ 5-membered heteroaryl containing one or two nitrogen atoms and one sulphur atom, wherein said heteroaryl is optionally substituted by X¹.
E4 A compound according to any of E3 wherein R¹ is a ‘C-linked’ 5-membered heteroaryl containing one or two nitrogen atoms and one sulphur atom.
E5 A compound according to E1 wherein R¹ is a ‘C-linked’ 5-membered heteroaryl containing one or two nitrogen atoms and one oxygen atom, wherein said heteroaryl is optionally substituted by one or two X¹.
E6 A compound according to E5 wherein R¹ is a ‘C-linked’ 5-membered heteroaryl containing one or two nitrogen atoms and one oxygen atom, wherein said heteroaryl is optionally substituted by X¹.
E7 A compound according to E6 wherein R¹ is a ‘C-linked’ 5-membered heteroaryl containing one nitrogen atom and one oxygen atom, wherein said heteroaryl is substituted by X¹.
E8 A compound according to any of E1 to E7 wherein each X¹ is independently selected from: F; Cl; and (C₁-C₄)alkyl optionally substituted by one, two or three F.
E9 A compound according to any of E1 to E8 wherein each X¹ is F, Cl or methyl.
E10 A compound according to any of E1 to E9 wherein R², R³ and R⁵ are independently selected from: H; halogen; CN; (C₁-C₃)alkyl; and (C₁-C₃)alkyloxy; and R⁴ is selected from: halogen; CN; (C₁-C₃)alkyl; and (C₁-C₃)alkyloxy.
E11 A compound according to any of E1 to E10 wherein R² and R³ are independently selected from H and F; R⁴ is halogen, CN or (C₁-C₃)alkyl; and R⁵ is H.
E12 A compound according to any of E1 to E11 wherein R² is H or F; R³ is H; R⁴ is Cl, Br, I, CN or (C₁-C₃)alkyl; and R⁵ is H.
E13 A compound according to any of E1 to E12 wherein R⁶ is phenyl substituted by one, two or three X².
E14 A compound according to E13 wherein R⁶ is phenyl substituted by two X².
E15 A compound according to any of E1 to E12 wherein R⁶ is ‘C-linked’ pyridinyl substituted by one, two or three X².
E16 A compound according to E15 wherein R⁶ is C-linked′ pyridinyl substituted by X².
E17 A compound according to any of E1 to E16 wherein each X² is independently selected from: halogen; CN; (C₁-C₄)alkyloxy optionally substituted by one, two or three F; (C₃-C₆)cycloalkyloxy; (C₁-C₄)alkyl optionally substituted by one, two or three F; and (C₁-C₄)alkyl substituted by OH.
E18 A compound according to E1 selected from:
4-(3-chloro-4-cyanophenoxy)-3-cyano-N-(1,3-thiazol-2-yl)benzenesulfonamide;
3-ethyl-4-(2-ethyl-4-fluorophenoxy)-N-(1,2,4-thiadiazol-5-yl)benzenesulfonamide;
4-(2-ethyl-4-fluorophenoxy)-3-iodo-N-(1,2,4-thiadiazol-5-yl)benzenesulfonamide;
3-cyano-4-(3,4-difluorophenoxy)-N-(1,3-thiazol-2-yl)benzenesulfonamide;
4-(3-chloro-4-cyanophenoxy)-3-cyano-N-(3-methyl-1,2-oxazol-4-yl)benzenesulfonamide;
4-(3-chloro-4-cyanophenoxy)-3-cyano-N-(1,3-thiazol-4-yl)benzenesulfonamide;
4-[4-chloro-2-(difluoromethoxy)phenoxy]-3-cyano-N-(1,3-thiazol-4-yl)benzenesulfonamide;
3-cyano-4-(2-ethyl-4-fluorophenoxy)-N-(1,3-thiazol-2-yl)benzenesulfonamide;
3-cyano-4-(3,4-difluorophenoxy)-N-(1,3-thiazol-4-yl)benzenesulfonamide;
3-cyano-4-[2-fluoro-4-(hydroxymethyl)phenoxy]-N-(1,3-thiazol-2-yl)benzenesulfonamide;
5-chloro-4-(3,4-difluoro-2-methylphenoxy)-2-fluoro-N-(1,3,4-thiadiazol-2-yl)benzenesulfonamide;
4-(2-ethyl-4-fluorophenoxy)-3-iodo-N-(1,3-thiazol-4-yl)benzenesulfonamide;
3-cyano-4-[(2-ethoxypyridin-3-yl)oxy]-N-(1,2,4-thiadiazol-5-yl)benzenesulfonamide;
5-bromo-4-(2-ethyl-4-fluorophenoxy)-2-fluoro-N-(1,3-thiazol-4-yl)benzenesulfonamide;
N-(5-chloro-1,3-thiazol-2-yl)-3-cyano-4-(3,4-difluorophenoxy)benzenesulfonamide; or
3-cyano-4-[3-cyano-5-(propan-2-yl)phenoxy]-N-(1,3-thiazol-2-yl)benzenesulfonamide;
   or a pharmaceutically acceptable salt thereof.
E19 The compound according to E1 selected from:
4-[3-chloro-4-(hydroxymethyl)phenoxy]-3-cyano-N-(1,3,4-thiadiazol-2-yl)benzenesulfonamide; or
3-cyano-4-(4-cyano-3,5-dimethylphenoxy)-N-(1,3,4-thiadiazol-2-yl)benzenesulfonamide;
   or a pharmaceutically acceptable salt thereof.

Alkyl and alkoxy groups, containing the requisite number of carbon atoms, can be unbranched or branched. Examples of alkyl include methyl, ethyl, n-propyl, i-propyl, n-butyl, i-butyl, sec-butyl and t-butyl. Examples of alkoxy include methoxy, ethoxy, n-propoxy, i-propoxy, n-butoxy, i-butoxy, sec-butoxy and t-butoxy.

Examples of cycloalkyl include cyclopropyl, cyclobutyl, cyclopentyl and cyclohexyl.

Halo means fluoro, chloro, bromo or iodo.

The term ‘C-linked’ used in the definitions of formula (I) means that the group in question is joined via a ring carbon.

Specific examples of ‘C-linked’ 5-membered heteroaryl containing one, two or three nitrogen atoms include pyrrolyl, pyrazolyl, imidazolyl, isoxazolyl, oxazolyl, isothiazolyl, thiazolyl, triazolyl oxadiazolyl and thiadiazolyl.

Specific examples of ‘C-linked’ 6-membered heteroaryl containing one or two nitrogen atoms include pyridinyl.

Hereinafter, all references to compounds of the invention include compounds of formula (I) or pharmaceutically acceptable salts, solvates, or multi-component complexes thereof, or pharmaceutically acceptable solvates or multi-component complexes of pharmaceutically acceptable salts of compounds of formula (I), as discussed in more detail below.

Preferred compounds of the invention are compounds of formula (I) or pharmaceutically acceptable salts thereof.

Suitable acid addition salts are formed from acids which form non-toxic salts. Examples include the acetate, adipate, aspartate, benzoate, besylate, bicarbonate/carbonate, bisulphate/sulphate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, pyroglutamate, saccharate, stearate, succinate, tannate, tartrate, tosylate, trifluoroacetate and xinofoate salts.

Suitable base salts are formed from bases which form non-toxic salts. Examples include the aluminium, arginine, benzathine, calcium, choline, diethylamine, diolamine, glycine, lysine, magnesium, meglumine, olamine, potassium, sodium, tromethamine and zinc salts.

Hemisalts of acids and bases may also be formed, for example, hemisulphate and hemicalcium salts.

The skilled person will appreciate that the aforementioned salts include ones wherein the counterion is optically active, for example d-lactate or 1-lysine, or racemic, for example dl-tartrate or dl-arginine.

For a review on suitable salts, see “Handbook of Pharmaceutical Salts: Properties, Selection, and Use” by Stahl and Wermuth (Wiley-VCH, Weinheim, Germany, 2002).

Pharmaceutically acceptable salts of compounds of formula (I) may be prepared by one or more of three methods:
(i) by reacting the compound of formula (I) with the desired acid or base;
(ii) by removing an acid- or base-labile protecting group from a suitable precursor of the compound of formula (I) using the desired acid or base; or
(iii) by converting one salt of the compound of formula (I) to another by reaction with an appropriate acid or base or by means of a suitable ion exchange column.

All three reactions are typically carried out in solution. The resulting salt may precipitate out and be collected by filtration or may be recovered by evaporation of the solvent. The degree of ionisation in the resulting salt may vary from completely ionised to almost non-ionised.

The compounds of formula (I) or pharmaceutically acceptable salts thereof may exist in both unsolvated and solvated forms. The term ‘solvate’ is used herein to describe a molecular complex comprising a compound of formula (I) or a pharmaceutically acceptable salt thereof and one or more pharmaceutically acceptable solvent molecules, for example, ethanol. The term ‘hydrate’ is employed when said solvent is water. Pharmaceutically acceptable solvates in accordance with the invention include those wherein the solvent of crystallization may be isotopically substituted, e.g. D₂O, d₆-acetone and d₆-DMSO.

A currently accepted classification system for organic hydrates is one that defines isolated site, channel, or metal-ion coordinated hydrates—see Polymorphism in Pharmaceutical Solids by K. R. Morris (Ed. H. G. Brittain, Marcel Dekker, 1995), incorporated herein by reference. Isolated site hydrates are ones in which the water molecules are isolated from direct contact with each other by intervening organic molecules. In channel hydrates, the water molecules lie in lattice channels where they are next to other water molecules. In metal-ion coordinated hydrates, the water molecules are bonded to the metal ion.

When the solvent or water is tightly bound, the complex will have a well-defined stoichiometry independent of humidity. When, however, the solvent or water is weakly bound, as in channel solvates and hygroscopic compounds, the water/solvent content will be dependent on humidity and drying conditions. In such cases, non-stoichiometry will be the norm.

The compounds of the invention may exist in a continuum of solid states ranging from fully amorphous to fully crystalline. The term ‘amorphous’ refers to a state in which the material lacks long range order at the molecular level and, depending upon temperature, may exhibit the physical properties of a solid or a liquid. Typically such materials do not give distinctive X-ray diffraction patterns and, while exhibiting the properties of a solid, are more formally described as a liquid. Upon heating, a change from solid to liquid properties occurs which is characterised by a change of state, typically second order (‘glass transition’). The term ‘crystalline’ refers to a solid phase in which the material has a regular ordered internal structure at the molecular level and gives a distinctive X-ray diffraction pattern with defined peaks. Such materials when heated sufficiently will also exhibit the properties of a liquid, but the change from solid to liquid is characterised by a phase change, typically first order (‘melting point’).

Also included within the scope of the invention are multi-component complexes (other than salts and solvates) of compounds of formula (I) or pharmaceutically acceptable salts thereof wherein the drug and at least one other component are present in stoichiometric or non-stoichiometric amounts. Complexes of this type include clathrates (drug-host inclusion complexes) and co-crystals. The latter are typically defined as crystalline complexes of neutral molecular constituents which are bound together through non-covalent interactions, but could also be a complex of a neutral molecule with a salt. Co-crystals may be prepared by melt crystallisation, by recrystallisation from solvents, or by physically grinding the components together—see Chem Commun, 17, 1889-1896, by O. Almarsson and M. J. Zaworotko (2004), incorporated herein by reference. For a general review of multi-component complexes, see J Pharm Sci, 64 (8), 1269-1288, by Haleblian (August 1975), incorporated herein by reference.

The compounds of the invention may also exist in a mesomorphic state (mesophase or liquid crystal) when subjected to suitable conditions. The mesomorphic state is intermediate between the true crystalline state and the true liquid state (either melt or solution). Mesomorphism arising as the result of a change in temperature is described as ‘thermotropic’ and that resulting from the addition of a second component, such as water or another solvent, is described as lyotropic′. Compounds that have the potential to form lyotropic mesophases are described as ‘amphiphilic’ and consist of molecules which possess an ionic (such as —COO⁻Na⁺, —COO⁻K⁺, or —SO₃⁻Na⁺) or non-ionic (such as —N⁻N⁺(CH₃)₃) polar head group. For more information, see Crystals and the Polarizing Microscope by N. H. Hartshorne and A. Stuart, 4^{th} Edition (Edward Arnold, 1970), incorporated herein by reference.

The compounds of the invention may be administered as prodrugs. Thus certain derivatives of compounds of formula (I) which may have little or no pharmacological activity themselves can, when administered into or onto the body, be converted into compounds of formula (I) having the desired activity, for example, by hydrolytic cleavage. Such derivatives are referred to as ‘prodrugs’. Further information on the use of prodrugs may be found in ‘Pro-drugs as Novel Delivery Systems, Vol. 14, ACS Symposium Series (T Higuchi and W Stella) and ‘Bioreversible Carriers in Drug Design’, Pergamon Press, 1987 (ed. E B Roche, American Pharmaceutical Association).

Prodrugs can, for example, be produced by replacing appropriate functionalities present in a compound of formula (I) with certain moieties known to those skilled in the art as ‘pro-moieties’ as described, for example, in “Design of Prodrugs” by H Bundgaard (Elsevier, 1985).

Examples of prodrugs include phosphate prodrugs, such as dihydrogen or dialkyl (e.g. di-tert-butyl) phosphate prodrugs. Further examples of replacement groups in accordance with the foregoing examples and examples of other prodrug types may be found in the aforementioned references.

Also included within the scope of the invention are metabolites of compounds of formula (I), that is, compounds formed in vivo upon administration of the drug. Some examples of metabolites in accordance with the invention include, where the compound of formula (I) contains a phenyl (Ph) moiety, a phenol derivative thereof (-Ph>-PhOH);

Compounds of the invention containing one or more asymmetric carbon atoms can exist as two or more stereoisomers. Included within the scope of the invention are all stereoisomers of the compounds of the invention and mixtures of one or more thereof.

Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC).

Alternatively, the racemate (or a racemic precursor) may be reacted with a suitable optically active compound, for example, an alcohol, or, in the case where the compound of formula (I) contains an acidic or basic moiety, a base or acid such as 1-phenylethylamine or tartaric acid. The resulting diastereomeric mixture may be separated by chromatography and/or fractional crystallization and one or both of the diastereoisomers converted to the corresponding pure enantiomer(s) by means well known to a skilled person.

Chiral compounds of the invention (and chiral precursors thereof) may be obtained in enantiomerically-enriched form using chromatography, typically HPLC, on an asymmetric resin with a mobile phase consisting of a hydrocarbon, typically heptane or hexane, containing from 0 to 50% by volume of isopropanol, typically from 2% to 20%, and from 0 to 5% by volume of an alkylamine, typically 0.1% diethylamine. Concentration of the eluate affords the enriched mixture.

Mixtures of stereoisomers may be separated by conventional techniques known to those skilled in the art; see, for example, “Stereochemistry of Organic Compounds” by E. L. Eliel and S. H. Wilen (Wiley, New York, 1994.

The scope of the invention includes all crystal forms of the compounds of the invention, including racemates and racemic mixtures (conglomerates) thereof. Stereoisomeric conglomerates may also be separated by the conventional techniques described herein just above.

The scope of the invention includes all pharmaceutically acceptable isotopically-labelled compounds of the invention wherein one or more atoms are replaced by atoms having the same atomic number, but an atomic mass or mass number different from the atomic mass or mass number which predominates in nature.

Examples of isotopes suitable for inclusion in the compounds of the invention include isotopes of hydrogen, such as ²H and ³H, carbon, such as ¹¹C, ¹³C and ¹⁴C, chlorine, such as ³⁶Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulphur, such as ³⁵S.

Certain isotopically-labelled compounds of the invention, for example, those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection. Substitution with heavier isotopes such as deuterium, i.e. ²H, may afford certain therapeutic advantages resulting from greater metabolic stability, for example, increased in vivo half-life or reduced dosage requirements, and hence may be preferred in some circumstances. Substitution with positron emitting isotopes, such as ¹¹C, ¹⁸F, ¹⁵O and ¹³N, can be useful in Positron Emission Topography (PET) studies for examining substrate receptor occupancy.

Isotopically-labeled compounds of formula (I) can generally be prepared by conventional techniques known to those skilled in the art or by processes analogous to those described in the accompanying Examples and Preparations using an appropriate isotopically-labeled reagent in place of the non-labeled reagent previously employed.

Also within the scope of the invention are intermediate compounds as hereinafter defined, all salts, solvates and complexes thereof and all solvates and complexes of salts thereof as defined hereinbefore for compounds of formula (I). The invention includes all polymorphs of the aforementioned species and crystal habits thereof.

When preparing a compound of formula (I) in accordance with the invention, a person skilled in the art may routinely select the form of intermediate which provides the best combination of features for this purpose. Such features include the melting point, solubility, processability and yield of the intermediate form and the resulting ease with which the product may be purified on isolation.

### BRIEF DESCRIPTION OF THE DRAWINGS

FIG. 1A represents a schematic showing organization of the URAT1(L)GFP construct (N to C terminal direction).

FIG. 1B represents a sequence alignment of the codon optimized URAT1(L)GFP construct with the wild type human URAT1 sequence deposited as NM_{—}144585.

Alignment row 1 is the sequence from accession NM_{—}144585.
Alignment row 2 is the sequence of the construct in the Gateway destination vector pLenti6.3V5/DEST (encoding URAT1(L)GFP) with the nucleotide alignment indicated with NM_{—}144585 above and the nucleotide numbering below.
Alignment row 3 is the amino acid translation with sequence annotation indicated in italics below.

The compounds of the invention may be prepared by any method known in the art for the preparation of compounds of analogous structure. In particular, the compounds of the invention can be prepared by the procedures described by reference to the Schemes that follow, or by the specific methods described in the Examples, or by similar processes to either.

The skilled person will appreciate that the experimental conditions set forth in the schemes that follow are illustrative of suitable conditions for effecting the transformations shown, and that it may be necessary or desirable to vary the precise conditions employed for the preparation of compounds of formula (I). It will be further appreciated that it may be necessary or desirable to carry out the transformations in a different order from that described in the schemes, or to modify one or more of the transformations, to provide the desired compound of the invention.

In addition, the skilled person will appreciate that it may be necessary or desirable at any stage in the synthesis of compounds of the invention to protect one or more sensitive groups, so as to prevent undesirable side reactions. In particular, it may be necessary or desirable to protect alcohol, amino or carboxylic acid groups. The protecting groups used in the preparation of the compounds of the invention may be used in conventional manner. See, for example, those described in ‘Greene's Protective Groups in Organic Synthesis’ by Theodora W Greene and Peter G M Wuts, fourth edition, (John Wiley and Sons, 2006), in particular chapters 1 (“Protection for the Hydroxyl Group . . . ”), 7 (“Protection for the Amino Group”) and 5 (“Protection for the Carboxyl Group”), incorporated herein by reference, which also describes methods for the removal of such groups.

Unless stated otherwise, in the following general processes R¹, R², R³, R⁴, R⁵ and R⁶ are as previously defined for a compound of formula (I); PG is a suitable amino protecting group, such as methoxymethyl, tert-butyl, tert-butyl carbamate, allyl or dimethoxybenzyl; and Hal is a suitable halogen, such as F or Cl. Where ratios of solvents are given, the ratios are by volume.

### DETAILED DESCRIPTION

According to a first process, compounds of formula (I) may be prepared from compounds of formula (II) and (III), as illustrated by Scheme 1.

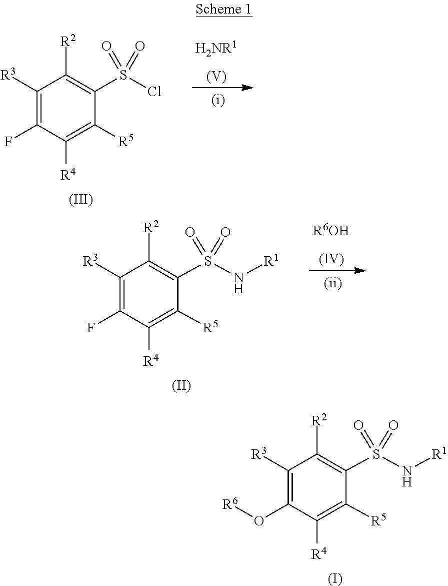

Compounds of formula (I) may be prepared from compounds of formula (II) according to reaction step (ii) by nucleophilic aromatic substitution reaction with compounds of formula (IV) under basic reaction conditions. Convenient conditions are potassium carbonate in DMF or DMSO; cesium carbonate in DMSO; or potassium phosphate in DMSO; and at from room temperature to elevated temperature. Typical conditions comprise potassium carbonate in DMSO at 80-100° C. for 18 hours.

Compounds of formula (II) may be prepared from compounds of formula (III) according to reaction step (i) by displacement of a sulfonyl chloride with compounds of formula (V) under basic reaction conditions. Convenient conditions are pyridine in DCM; 1,4-diazabicyclo[2.2.2]octane in acetonitrile; N-methylmorpholine in THF; or an excess of compound of formula (V). Preferred conditions comprise pyridine in DCM at room temperature.

According to a second process, compounds of formula (I) may be prepared from compounds of formulae (II) and (VIII), as illustrated by Scheme 2.

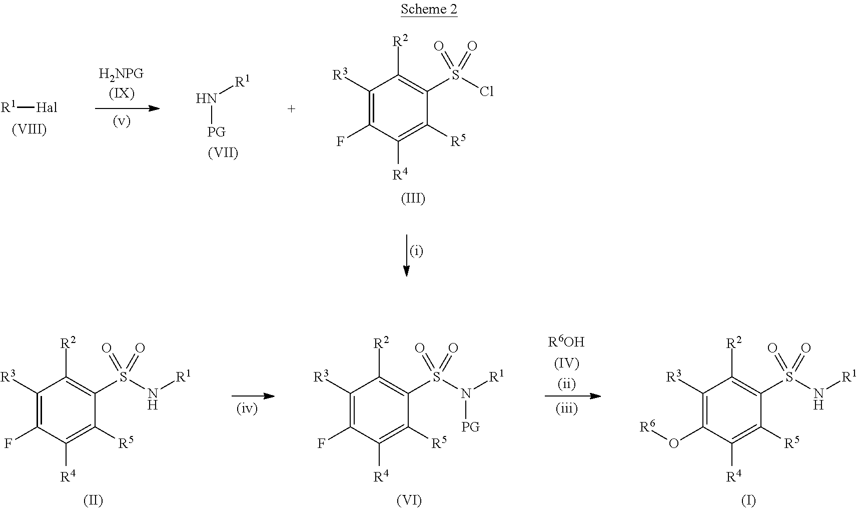

Compounds of formula (I) may be prepared from compound of formula (VI) according to process step (ii), under the conditions described in Scheme 1 step (ii), followed by deprotection step (iii), typically mediated by an inorganic or organic acid. Preferred conditions comprise potassium carbonate in DMSO at room temperature, followed by trifluoroacetic acid in DCM or HCl in 1,4-dioxane. It is also possible that deprotection step (iii) may occur under the conditions for effecting the nucleophilic aromatic substitution of step (ii).

Compounds of formula (VI) may be prepared from compounds of formula (II) according to process step (iv) by introduction of a suitable protecting group, such as tert-butyl, tert-butyl carbamate, allyl or dimethoxybenzyl, under basic reaction conditions or Mitsunobu reaction conditions. Typical conditions comprise potassium carbonate in THF at room temperature.

Alternatively, compounds of formula (VI) may be prepared from compounds of formula (III) according to process step (i) according to the conditions described in Scheme 1 step (i), or by using sodium or lithium hexamethyldisilazane in THF at from −78° C. to room temperature.

Compounds of formulae (II) and (III) may be prepared as described in Scheme 1.

Compounds of formula (VII) may be prepared from compounds of formula (VIII) according to reaction step (v) by a nucleophilic aromatic substitution reaction with compounds of formula (IX) under basic reaction conditions. Preferred conditions comprise diisopropylethylamine in n-butanol at 100° C. or potassium carbonate in DMSO at 110° C.

According to a third process, compounds of formula (I) may be prepared from compounds of formula (XIII) as illustrated by Scheme 3.

Compounds of formula (I) may be prepared from compounds of formula (X) according to the conditions described in Scheme 2, step (i).

Compounds of formula (X) may be prepared from compounds of formula (XI) according to process step (vii), an oxidation reaction in the presence of trichloroisocyanuric acid. Preferred conditions comprise trichloroisocyanuric acid with benzyltrimethylammonium chloride and sodium carbonate in acetonitrile and water.

Compounds of formula (XI) may be prepared from compounds of formula (XII) according to process step (ii), a nucleophilic aromatic substitution reaction with compounds of formula (IV) as described in Scheme 1, step (i).

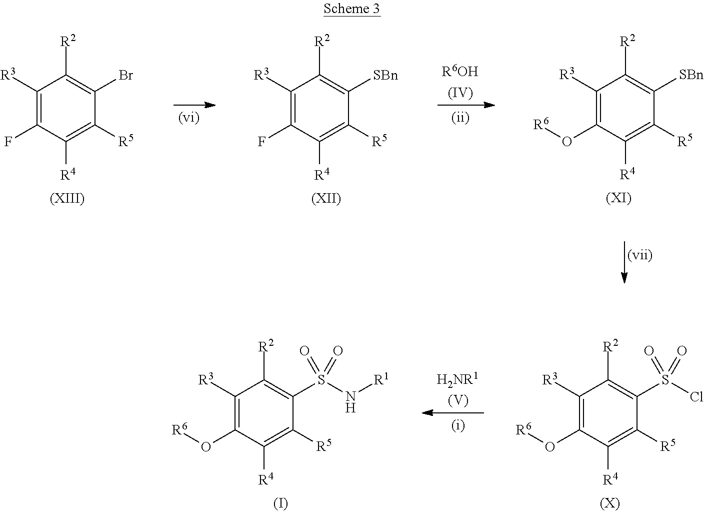

Compounds of formula (XII) may be prepared from compounds of formula (XIII) according to process step (vi), a cross-coupling reaction with benzylmercaptan in the presence of a suitable catalyst. Conveniently the catalyst is a palladium catalyst. Preferred conditions comprise diisopropylethyamine with [1,1-bis(di-tert-butylphosphino)]ferrocene palladium (II) in toluene at 60° C.

Compounds of formula (X) may also be prepared from compounds of formula (XV) as illustrated by Scheme 4.

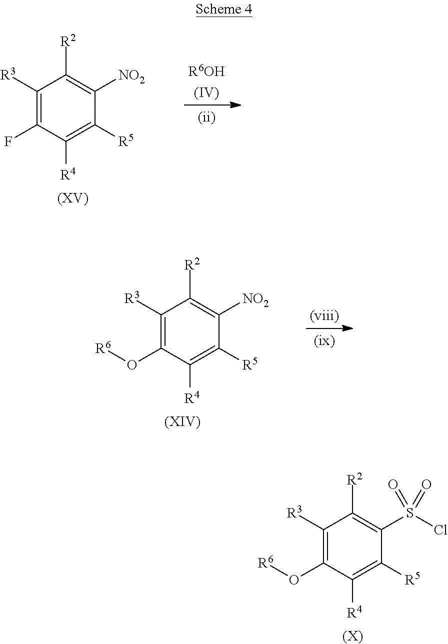

Compounds of formula (X) may be prepared from compounds of formula (XIV) according to process steps (viii), a reduction reaction, followed by process step (ix), a Sandmeyer reaction. Conveniently the reduction may be effected by hydrogenation, use of a suitable metal reducing agent or use of sodium dithionite. Suitable reduction conditions comprise iron powder and calcium chloride in ethanol/water. Suitable Sandmeyer reaction conditions include sodium nitrite in HCl, acetic acid and water, with the addition of sulfur dioxide in acetic acid and copper chloride at 0° C.

Compounds of formula (XIV) may be prepared from compounds of formula (XII) according to process step (ii), under the conditions described in Scheme 1 step (ii).

According to a fifth process, compounds of formula (I) may be prepared from compounds of formula (XVI) as illustrated by Scheme 5.

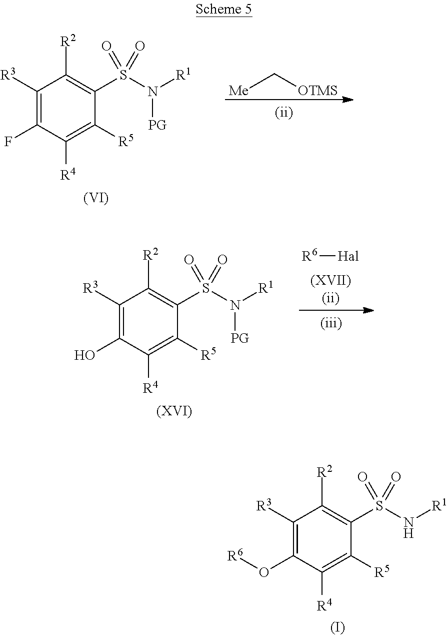

Wherein Hal is a suitable halogen such as Cl, Br.

Compounds of formula (I) may be prepared from compounds of formula (XVI) according to process step (ii) under the conditions described in Scheme 1 (step (ii), followed by deprotection step (iii) under the conditions described in Scheme 2 step (iii). Convenient process step (ii) conditions include sodium hydride in DMF or DMSO, at from room temperature to 100° C.

Compounds of formula (XVI) may be prepared from compounds of formula (VI) according to process step (ii), a nucleophilic aromatic substitution reaction with trimethylsilylethanol, under the conditions described in Scheme 1.

Compounds of formula (II) wherein R¹ is

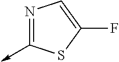

may also be prepared by fluorination of the corresponding des-fluoro compounds of formula (II) as illustrated by Scheme 6.

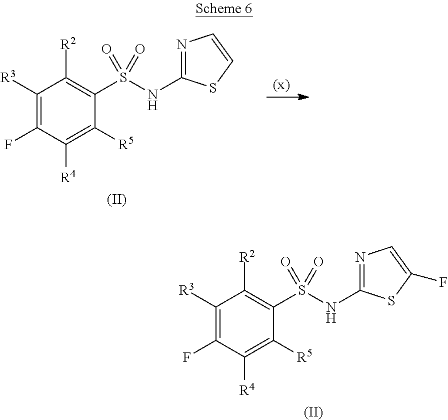

Convenient step (x) fluorination conditions comprise Selectfluor™ in MeCN/water, followed by dehydration using triethylamine and acetic anhydride in DCM at room temperature.

Compounds of formula (I) wherein R⁶ is

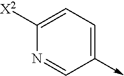

may also be prepared from the corresponding compounds of formula (I) wherein X² is F by nucleophilic aromatic substitution, as illustrated by Scheme 7.

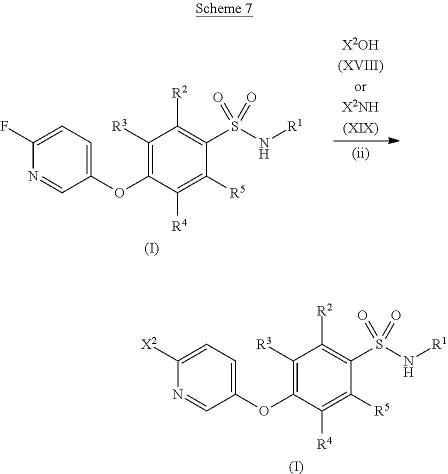

The step (ii) nucleophilic aromatic substitution reaction may be effected under the conditions described in Scheme 1 step (ii). Preferred conditions comprise an excess of compounds of formula (XIX) in DMF at 90° C., or sodium hydride in THF at room temperature.

Compounds of formula (I) wherein R⁶ is

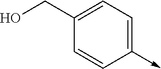

may also be prepared by reduction of the corresponding aldehydes of formula (XX), as illustrated by Scheme 8.

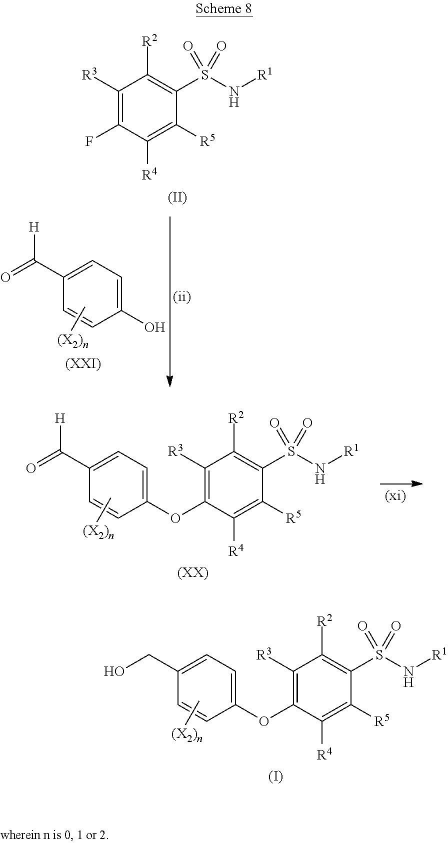

Compounds of formula (I) may be prepared from compounds of formula (XX) by reduction according to process steps (xi). Convenient step (xi) reduction conditions comprise sodium borohydride in methanol at room temperature.

Compounds of formula (XX) may be prepared from compounds of formula (II) by nucleophilic aromatic substitution according to process steps (ii). This nucleophilic aromatic substitution reaction may be effected under the conditions described in Scheme 1 step (ii). Preferably the reaction is carried out in an excess of compound of formula (XXI), a solvent such as DMSO, a base such as potassium phosphate and at elevated temperature, such as about 100° C.

The skilled person will appreciate that a compound of formula (I) wherein R², R³, R⁴ or R⁵ is Cl, Br or I may be converted into the corresponding compound of formula (I) wherein the group in question is H, by dehalogenation in the presence of a suitable catalyst. Typical conditions comprise zinc dust in acetic acid at room temperature.

Compounds of formula (III), (IV), (V), (VIII), (IX), (XIII), (XV), (XVII), (XVIII), (XIX) and (XXI) are commercially available, known from the literature, easily prepared by methods well known to those skilled in the art, or can be made according to preparations described herein.

All new processes for preparing compounds of formula (I), and corresponding new intermediates employed in such processes, form further aspects of the present invention.

Compounds of the invention intended for pharmaceutical use may be administered as crystalline or amorphous products or may exist in a continuum of solid states ranging from fully amorphous to fully crystalline. They may be obtained, for example, as solid plugs, powders, or films by methods such as precipitation, crystallization, freeze drying, spray drying, or evaporative drying. Microwave or radio frequency drying may be used for this purpose.

They may be administered alone or in combination with one or more other compounds of the invention or in combination with one or more other drugs (or as any combination thereof). Generally, they will be administered as a formulation in association with one or more pharmaceutically acceptable excipients. The term ‘excipient’ is used herein to describe any ingredient other than the compound(s) of the invention. The choice of excipient will to a large extent depend on factors such as the particular mode of administration, the effect of the excipient on solubility and stability, and the nature of the dosage form.

In another aspect the invention provides a pharmaceutical composition comprising a compound of the invention together with one or more pharmaceutically acceptable excipients.

Pharmaceutical compositions suitable for the delivery of compounds of the present invention and methods for their preparation will be readily apparent to those skilled in the art. Such compositions and methods for their preparation may be found, for example, in “Remington's Pharmaceutical Sciences”, 19th Edition (Mack Publishing Company, 1995).

Suitable modes of administration include oral, parenteral, topical, inhaled/intranasal, rectal/intravaginal, and ocular/aural administration.

Formulations suitable for the aforementioned modes of administration may be formulated to be immediate and/or modified release. Modified release formulations include delayed-, sustained-, pulsed-, controlled-, targeted and programmed release.

The compounds of the invention may be administered orally. Oral administration may involve swallowing, so that the compound enters the gastrointestinal tract, or buccal or sublingual administration may be employed by which the compound enters the blood stream directly from the mouth. Formulations suitable for oral administration include solid formulations such as tablets, capsules containing particulates, liquids, or powders, lozenges (including liquid-filled), chews, multi- and nano-particulates, gels, solid solution, liposome, films, ovules, sprays, liquid formulations and buccal/mucoadhesive patches.

Liquid formulations include suspensions, solutions, syrups and elixirs. Such formulations may be employed as fillers in soft or hard capsules and typically comprise a carrier, for example, water, ethanol, polyethylene glycol, propylene glycol, methylcellulose, or a suitable oil, and one or more emulsifying agents and/or suspending agents. Liquid formulations may also be prepared by the reconstitution of a solid, for example, from a sachet.

The compounds of the invention may also be used in fast-dissolving, fast-disintegrating dosage forms such as those described in Expert Opinion in Therapeutic Patents, 11 (6), 981-986, by Liang and Chen (2001).

For tablet dosage forms, depending on dose, the drug may make up from 1 weight % to 80 weight % of the dosage form, more typically from 5 weight % to 60 weight % of the dosage form. In addition to the drug, tablets generally contain a disintegrant. Examples of disintegrants include sodium starch glycolate, sodium carboxymethyl cellulose, calcium carboxymethyl cellulose, croscarmellose sodium, crospovidone, polyvinylpyrrolidone, methyl cellulose, microcrystalline cellulose, lower alkyl-substituted hydroxypropyl cellulose, starch, pregelatinised starch and sodium alginate. Generally, the disintegrant will comprise from 1 weight % to 25 weight %, preferably from 5 weight % to 20 weight % of the dosage form.

Binders are generally used to impart cohesive qualities to a tablet formulation. Suitable binders include microcrystalline cellulose, gelatin, sugars, polyethylene glycol, natural and synthetic gums, polyvinylpyrrolidone, pregelatinised starch, hydroxypropyl cellulose and hydroxypropyl methylcellulose. Tablets may also contain diluents, such as lactose (monohydrate, spray-dried monohydrate, anhydrous and the like), mannitol, xylitol, dextrose, sucrose, sorbitol, microcrystalline cellulose, starch and dibasic calcium phosphate dihydrate.

Tablets may also optionally comprise surface active agents, such as sodium lauryl sulfate and polysorbate 80, and glidants such as silicon dioxide and talc. When present, surface active agents may comprise from 0.2 weight % to 5 weight % of the tablet, and glidants may comprise from 0.2 weight % to 1 weight % of the tablet.

Tablets also generally contain lubricants such as magnesium stearate, calcium stearate, zinc stearate, sodium stearyl fumarate, and mixtures of magnesium stearate with sodium lauryl sulphate. Lubricants generally comprise from 0.25 weight % to 10 weight %, preferably from 0.5 weight % to 3 weight % of the tablet. Other possible ingredients include anti-oxidants, colourants, flavouring agents, preservatives and taste-masking agents.

Exemplary tablets contain up to about 80% drug, from about 10 weight % to about 90 weight % binder, from about 0 weight % to about 85 weight % diluent, from about 2 weight % to about 10 weight % disintegrant, and from about 0.25 weight % to about 10 weight % lubricant. Tablet blends may be compressed directly or by roller to form tablets. Tablet blends or portions of blends may alternatively be wet-, dry-, or melt-granulated, melt congealed, or extruded before tabletting. The final formulation may comprise one or more layers and may be coated or uncoated; it may even be encapsulated. The formulation of tablets is discussed in “Pharmaceutical Dosage Forms: Tablets”, Vol. 1, by H. Lieberman and L. Lachman (Marcel Dekker, New York, 1980).

Suitable modified release formulations for the purposes of the invention are described in U.S. Pat. No. 6,106,864. Details of other suitable release technologies such as high energy dispersions and osmotic and coated particles are to be found in “Pharmaceutical Technology On-line”, 25(2), 1-14, by Verma et al (2001). The use of chewing gum to achieve controlled release is described in WO 00/35298.

The compounds of the invention may also be administered directly into the blood stream, into muscle, or into an internal organ. Suitable means for parenteral administration include intravenous, intraarterial, intraperitoneal, intrathecal, intraventricular, intraurethral, intrasternal, intracranial, intramuscular and subcutaneous. Suitable devices for parenteral administration include needle (including microneedle) injectors, needle-free injectors and infusion techniques.

Parenteral formulations are typically aqueous solutions which may contain excipients such as salts, carbohydrates and buffering agents (preferably to a pH of from 3 to 9), but, for some applications, they may be more suitably formulated as a sterile non-aqueous solution or as a dried form to be used in conjunction with a suitable vehicle such as sterile, pyrogen-free water.

The preparation of parenteral formulations under sterile conditions, for example, by lyophilisation, may readily be accomplished using standard pharmaceutical techniques well known to those skilled in the art.

The solubility of compounds of formula (I) used in the preparation of parenteral solutions may be increased by the use of appropriate formulation techniques, such as the incorporation of solubility-enhancing agents. Formulations for parenteral administration may be formulated to be immediate and/or modified release. Modified release formulations include delayed-, sustained-, pulsed-, controlled-, targeted and programmed release. Thus compounds of the invention may be formulated as a solid, semi-solid, or thixotropic liquid for administration as an implanted depot providing modified release of the active compound. Examples of such formulations include drug-coated stents and poly(dl-lactic-coglycolic)acid (PGLA) microspheres.

The compounds of the invention may also be administered topically to the skin or mucosa, that is, dermally or transdermally. Typical formulations for this purpose include gels, hydrogels, lotions, solutions, creams, ointments, dusting powders, dressings, foams, films, skin patches, wafers, implants, sponges, fibres, bandages and microemulsions. Liposomes may also be used. Typical carriers include alcohol, water, mineral oil, liquid petrolatum, white petrolatum, glycerin, polyethylene glycol and propylene glycol. Penetration enhancers may be incorporated—see, for example, J Pharm Sci, 88 (10), 955-958, by Finnin and Morgan (October 1999).

Other means of topical administration include delivery by electroporation, iontophoresis, phonophoresis, sonophoresis and microneedle or needle-free (e.g. Powderject™ Bioject™, etc.) injection.

The compounds of the invention can also be administered intranasally or by inhalation, typically in the form of a dry powder (either alone, as a mixture, for example, in a dry blend with lactose, or as a mixed component particle, for example, mixed with phospholipids, such as phosphatidylcholine) from a dry powder inhaler or as an aerosol spray from a pressurised container, pump, spray, atomiser (preferably an atomiser using electrohydrodynamics to produce a fine mist), or nebuliser, with or without the use of a suitable propellant, such as 1,1,1,2-tetrafluoroethane or 1,1,1,2,3,3,3-heptafluoropropane. For intranasal use, the powder may comprise a bioadhesive agent, for example, chitosan or cyclodextrin.

The pressurised container, pump, spray, atomizer, or nebuliser contains a solution or suspension of the compound(s) of the invention comprising, for example, ethanol, aqueous ethanol, or a suitable alternative agent for dispersing, solubilising, or extending release of the active, a propellant(s) as solvent and an optional surfactant, such as sorbitan trioleate, oleic acid, or an oligolactic acid.

Prior to use in a dry powder or suspension formulation, the drug product is micronised to a size suitable for delivery by inhalation (typically less than 5 microns). This may be achieved by any appropriate comminuting method, such as spiral jet milling, fluid bed jet milling, supercritical fluid processing to form nanoparticles, high pressure homogenisation, or spray drying.

Capsules (made, for example, from gelatin or hydroxypropylmethylcellulose), blisters and cartridges for use in an inhaler or insufflator may be formulated to contain a powder mix of the compound of the invention, a suitable powder base such as lactose or starch and a performance modifier such as 1-leucine, mannitol, or magnesium stearate. The lactose may be anhydrous or in the form of the monohydrate, preferably the latter. Other suitable excipients include dextran, glucose, maltose, sorbitol, xylitol, fructose, sucrose and trehalose.

A suitable solution formulation for use in an atomiser using electrohydrodynamics to produce a fine mist may contain from 1 μg to 20 mg of the compound of the invention per actuation and the actuation volume may vary from 1 μl to 100 μl. A typical formulation may comprise a compound of formula (I), propylene glycol, sterile water, ethanol and sodium chloride. Alternative solvents which may be used instead of propylene glycol include glycerol and polyethylene glycol.

Suitable flavours, such as menthol and levomenthol, or sweeteners, such as saccharin or saccharin sodium, may be added to those formulations of the invention intended for inhaled/intranasal administration.

In the case of dry powder inhalers and aerosols, the dosage unit is determined by means of a valve which delivers a metered amount. Units in accordance with the invention are typically arranged to administer a metered dose or “puff” containing from 1 μg to 100 mg of the compound of formula (I). The overall daily dose will typically be in the range 1 μg to 200 mg which may be administered in a single dose or, more usually, as divided doses throughout the day.

The compounds of the invention may be administered rectally or vaginally, for example, in the form of a suppository, pessary, microbicide, vaginal ring or enema. Cocoa butter is a traditional suppository base, but various alternatives may be used as appropriate.

The compounds of the invention may also be administered directly to the eye or ear, typically in the form of drops of a micronised suspension or solution in isotonic, pH-adjusted, sterile saline. Other formulations suitable for ocular and aural administration include ointments, biodegradable (e.g. absorbable gel sponges, collagen) and non-biodegradable (e.g. silicone) implants, wafers, lenses and particulate or vesicular systems, such as niosomes or liposomes. A polymer such as crossed-linked polyacrylic acid, polyvinylalcohol, hyaluronic acid, a cellulosic polymer, for example, hydroxypropylmethylcellulose, hydroxyethylcellulose, or methyl cellulose, or a heteropolysaccharide polymer, for example, gelan gum, may be incorporated together with a preservative, such as benzalkonium chloride. Such formulations may also be delivered by iontophoresis.

The compounds of the invention may be combined with soluble macromolecular entities, such as cyclodextrin and suitable derivatives thereof or polyethylene glycol-containing polymers, in order to improve their solubility, dissolution rate, taste-masking, bioavailability and/or stability for use in any of the aforementioned modes of administration.

Drug-cyclodextrin complexes, for example, are found to be generally useful for most dosage forms and administration routes. Both inclusion and non-inclusion complexes may be used. As an alternative to direct complexation with the drug, the cyclodextrin may be used as an auxiliary additive, i.e. as a carrier, diluent, or solubiliser. Most commonly used for these purposes are alpha-, beta- and gamma-cyclodextrins, examples of which may be found in International Patent Applications Nos. WO 91/11172, WO 94/02518 and WO 98/55148.

For administration to human patients, the total daily dose of the compounds of the invention is typically in the range 1 mg to 10 g, such as 10 mg to 1 g, for example 25 mg to 500 mg depending, of course, on the mode of administration and efficacy. For example, oral administration may require a total daily dose of from 50 mg to 100 mg. The total daily dose may be administered in single or divided doses and may, at the physician's discretion, fall outside of the typical range given herein. These dosages are based on an average human subject having a weight of about 60 kg to 70 kg. The physician will readily be able to determine doses for subjects whose weight falls outside this range, such as infants and the elderly.

As noted above, the compounds of the invention are useful because they exhibit pharmacological activity in animals, i.e., URAT-1 inhibition. More particularly, the compounds of the invention are of use in the treatment of disorders for which a URAT-1 inhibitor is indicated. Preferably the animal is a mammal, more preferably a human.

In a further aspect of the invention there is provided a compound of the invention for use as a medicament.

In a further aspect of the invention there is provided a compound of the invention for the treatment of a disorder for which a URAT-1 inhibitor is indicated.

In a further aspect of the invention there is provided use of a compound of the invention for the preparation of a medicament for the treatment of a disorder for which a URAT-1 inhibitor is indicated.

In a further aspect of the invention there is provided a method of treating a disorder in an animal (preferably a mammal, more preferably a human) for which a URAT-1 inhibitor is indicated, comprising administering to said animal a therapeutically effective amount of a compound of the invention.

Disorders for which a URAT-1 inhibitor is indicated include diseases associated with high levels of uric acid in humans and other mammals including (but not limited to) hyperuricemia, asymptomatic hyperuricemia, gout (including juvenile forms), gouty arthritis, inflammatory arthritis, joint inflammation, deposition of urate crystals in the joint, tophaceous gout, chronic kidney disease, nephrolithiasis (kidney stones), Lesch-Nyhan syndrome and Kelley-Seegmiller syndrome.

Hyperuricemia may be defined by blood uric acid levels over 6.8 mg/dL. Guidelines for the management of hyperuricemia recommend that therapies aimed at lowering blood uric acid levels should be maintained until such blood uric acid levels are lowered to below 6.0 mg/dL, such as below 5.0 mg/dL.

The skilled person will appreciate that while by definition without symptoms, asymptomatic hyperuricemia may nevertheless lead to the onset of diseases associated with high levels of uric acid.

The skilled person will also appreciate that the compounds of formula (I) may be used in the treatment of hyperuricemia where this is present together with one or more other diseases, such as kidney failure, type 2 diabetes, cardiovascular disease (e.g. hypertension, myocardial infarction, heart failure, coronary artery disease, cerebrovascular disease, atherosclerosis, angina, aneurism, hyperlipidemia and stroke), obesity, metabolic syndrome, myeloproliferative disorders, lymphoproliferative disorders and disorders associated with certain medications, such as a diuretic (e.g. a thiazide), an immunosuppressant (e.g. a cyclosporine therapy), a chemotherapeutic agent (e.g. cisplatin) or aspirin.

The skilled person will also appreciate that the compounds of formula (I) may be used in the treatment of hyperuricemia where this is present following organ transplant.

A URAT-1 inhibitor may be usefully combined with another pharmacologically active compound, or with two or more other pharmacologically active compounds, particularly in the treatment of a disease associated with elevated blood uric acid levels. Such combinations offer the possibility of significant advantages, including patient compliance, ease of dosing and synergistic activity.

In the combinations that follow the compound of the invention may be administered simultaneously, sequentially or separately in combination with the other therapeutic agent or agents.

The compounds of formula (I) may be administered in combination with one or more additional therapeutic agents. Agents of interest include those that also lower blood uric acid levels. Other agents of interest include those that reduce inflammation or pain. The one or more additional therapeutic agents may be selected from any of the agents or types of agent that follow:
a xanthine oxidase inhibitor (e.g. allopurinol, febuxostat or tisopurine);
a purine nucleoside phosphorylase (PNP) inhibitor (e.g. ulodesine);
a uricase (e.g. pegloticase or rasburicase);
a uricosuric, such as an agent that inhibits one or more transporters responsible for reabsorption of uric acid back into the blood at renal or intestinal sites, for example another URAT1 inhibitor (e.g. benzbromarone, PN2107 or RDEA3170); a glucose transporter (GLUT) inhibitor, such as a GLUT9 inhibitor; an organic anion transporter (OAT) inhibitor, such as an OAT4 inhibitor or an OAT10 inhibitor; or an agent which inhibits one or more of the above transporters, such as benziodarone; isobromindione, probenecid, sulphinpyrazone, arhalofenate, tranilast, lesinurad or KUX-1151;
an agent that otherwise exerts blood uric acid lowering effects, such as amlodipine, atorvastatin, fenofibrate or indomethacin;
an anti-inflammatory drug such as an NSAID (e.g. celecoxib), colchicine, a steroid, an interleukin 1 inhibitor (e.g. rilonacept) or an agent that modulates inflammosome signaling cascades (e.g. an IRAK4 inhibitor); or

an agent that reduces pain, such as an ion channel modulator (e.g. an inhibitor of Nav1.7, TRPV1 or TRPM2).

There is also included within the scope the present invention combinations of a compound of the invention together with one or more additional therapeutic agents which slow down the rate of metabolism of the compound of the invention, thereby leading to increased exposure in patients. Increasing the exposure in such a manner is known as boosting. This has the benefit of increasing the efficacy of the compound of the invention or reducing the dose required to achieve the same efficacy as an unboosted dose. The metabolism of the compounds of the invention includes oxidative processes carried out by P450 (CYP450) enzymes, particularly CYP 3A4 and conjugation by UDP glucuronosyl transferase and sulphating enzymes. Thus, among the agents that may be used to increase the exposure of a patient to a compound of the present invention are those that can act as inhibitors of at least one isoform of the cytochrome P450 (CYP450) enzymes. The isoforms of CYP450 that may be beneficially inhibited include, but are not limited to, CYP1A2, CYP2D6, CYP2C9, CYP2C19 and CYP3A4. Suitable agents that may be used to inhibit CYP 3A4 include ritonavir, saquinavir, ketoconazole, N-(3,4-difluorobenzyl)-N-methyl-2-{[(4-methoxypyridin-3-yl)amino]sulfonyl}benzamide and N-(1-(2-(5-(4-fluorobenzyl)-3-(pyridin-4-yl)-1H-pyrazol-1-yl)acetyl)piperidin-4-yl)methanesulfonamide.

It is within the scope of the invention that two or more pharmaceutical compositions, at least one of which contains a compound of the invention, may conveniently be combined in the form of a kit suitable for coadministration of the compositions. Thus the kit of the invention comprises two or more separate pharmaceutical compositions, at least one of which contains a compound of the invention, and means for separately retaining said compositions, such as a container, divided bottle, or divided foil packet. An example of such a kit is the familiar blister pack used for the packaging of tablets, capsules and the like. The kit of the invention is particularly suitable for administering different dosage forms, for example, oral and parenteral, for administering the separate compositions at different dosage intervals, or for titrating the separate compositions against one another. To assist compliance, the kit typically comprises directions for administration and may be provided with a so-called memory aid.

In another aspect the invention provides a pharmaceutical product (such as in the form of a kit) comprising a compound of the invention together with one or more additional therapeutically active agents as a combined preparation for simultaneous, separate or sequential use in the treatment of a disorder for which a URAT-1 inhibitor is indicated.

It is to be appreciated that all references herein to treatment include curative, palliative and prophylactic treatment.

In the non-limiting Examples and Preparations that are set out later in the description, and in the aforementioned Schemes, the following the abbreviations, definitions and analytical procedures may be referred to:

AcOH is acetic acid;

Boc is tert-butyl carbamate;

nBuOH is n-butanol;

CD₃I is iodomethane-d₃;

Cu(acac)₂ is copper (II) acetylacetonate;

Cu(OAc)₂ is copper (II) acetate;

DABCO is 1,4-diazabicyclo[2,2,2]octane

DAD is diode array detector;

DCM is dichloromethane; methylene chloride;

DEA is diethylamine

DIAD is diisopropyl azodicarboxylate;

DIP-Cl is chlorodiisopinocampheylborane;

DIPEA is N-ethyldiisopropylamine, N,N-diisopropylethylamine;

DMAP is 4-dimethylaminopyridine;

DMF is N,N-dimethylformamide;

DMSO is dimethyl sulphoxide;

EDCl is 1-(3-dimethylaminopropyl)-3-ethylcarbodiimide hydrochloride;

EDTA is ethylenediaminetetraacetic acid;

ELSD is evaporative light scattering detection;

EtOAc is ethyl acetate;

HPLC is high-performance liquid chromatography

IPA is isopropanol;

Ir₂(OMe)₂COD₂ is bis(1,5-cyclooctadiene)di-μ-methoxydiiridium (I);

KOAc is potassium acetate;

K₃PO₄ is potassium phosphate tribasic;

LCMS is liquid chromatography mass spectrometry (Rₜ=retention time)

LiHMDS is lithium bis(trimethylsilyl)amide;

Me is methyl

MeCN is acetonitrile

MeOH is methanol;

MS is mass spectrometry

NaHMDS is sodium bis(trimethylsilyl)amide

NMM is N-methylmorpholine

NMP is N-Methyl-2-pyrrolidone;

Pd/C is palladium on carbon;

Pd(PPh₃)₄ is palladium tetrakis;

Pd(dppf)₂Cl₂ is [1,1′-bis(diphenylphosphino)ferrocene]dichloropalladium(II), complex with dichloromethane;

TBAF is tetra-n-butylammonium fluoride

TBME is tert-butyl methy ether;

TFA is trifluoroacetate;

THF is tetrahydrofuran;

THP is tetrahydropyran;

TLC is thin layer chromatography;

UV is ultraviolet; and

WSCDI is 1-(3-dimethylaminopropyl)-3-ethylcarbodiimide hydrochloride.

¹H and ¹⁹F Nuclear magnetic resonance (NMR) spectra were in all cases consistent with the proposed structures. Characteristic chemical shifts (δ) are given in parts-per-million downfield from tetramethylsilane (for ¹H-NMR) and upfield from trichloro-fluoro-methane (for ¹⁹F NMR) using conventional abbreviations for designation of major peaks: e.g. s, singlet; d, doublet; t, triplet; q, quartet; m, multiplet; br, broad. The following abbreviations have been used for common solvents: CDCl₃, deuterochloroform; d₆-DMSO, deuterodimethylsulphoxide; and CD₃OD, deuteromethanol.

Mass spectra, MS (m/z), were recorded using either electrospray ionisation (ESI) or atmospheric pressure chemical ionisation (APCI).

Where relevant and unless otherwise stated the m/z data provided are for isotopes ¹⁹F, ³⁵Cl, ⁷⁹Br and ¹²⁷I.

### LCMS Conditions:

### System 1

A: 0.1% formic acid in water

B: 0.1% formic acid in acetonitrile

Column: C18 phase Phenomenex 20×4.0 mm with 3 micron particle size

Gradient: 98-2% or 98-10% A over 1.5 min, 0.3 min hold, 0.2 re-equilibration, 1.8 mL/min flow rate

UV: 210 nm-450 nm DAD

Temperature: 75° C.

### System 2

A: 0.1% formic acid in water

B: 0.1% formic acid in acetonitrile

Using Either:

Column: Agilent Extend C18 phase 50×3 mm with 3 micron particle size

Gradient: 95-0% A over 3.5 min, 1 min hold, 0.4 min re-equilibration, 1.2 mL/min flow rate
Or

Column: C18 phase Waters Sunfire 50×4.6 mm with 5 micron particle size

Gradient: 95-5% A over 3 min, 1 min hold, 2 min re-equilibration, 1 mL/min flow rate

UV: 210 nm-450 nm DAD

Temperature: 50° C.

### System 3

A: 10 mM Ammonium Acetate in water (basic Buffer)

B: Acetonitrile

Column: Xbridge C18 4.6×50 mm with 5 micron particle size

Gradient: from 90% [Buffer] and 10% [MeCN] to 70% [Buffer] and 30% [MeCN] in 1.5 min, further to 10% [buffer] and 90% [MeCN] in 3.0 min, held for 4 min and back to initial condition in 5 min),

1.2 mL/minflow rate

UV: 220 nm

Temperature: 25° C.

### DETAILED DESCRIPTION OF THE PREFERRED EMBODIMENTS

### Example 1

### 4-(3-chloro-4-cyanophenoxy)-3-cyano-N-(1,3-thiazol-2-yl)benzenesulfonamide

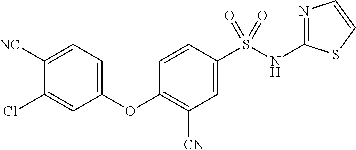 [SMILES: N#Cc1ccc(Oc2ccc(S(=O)(=O)Nc3nccs3)cc2C#N)cc1Cl]

To a solution of 3-cyano-4-fluoro-N-(1,3-thiazol-2-yl)benzenesulfonamide (WO2012004743, 200 mg, 0.71 mmol) and 3-chloro-4-cyanophenol (163 mg, 1.06 mmol) in DMSO (4 mL) was added potassium carbonate (293 mg, 2.21 mmol) and the reaction mixture heated to 60° C. for 18 hours followed by heating at 80° C. for a further 4 hours. The reaction mixture was cooled, diluted with EtOAc (50 mL) and washed with saturated aqueous NaHCO₃ solution (2×40 mL) and brine (30 mL). The organic layer was collected, dried over MgSO₄ and concentrated in vacuo. The residue was purified using silica gel column chromatography eluting with a gradient of between 0-80% EtOAc in heptanes to afford the title compound as a cream solid (62 mg, 21%).

¹H NMR (400 MHz, DMSO-d₆): δ ppm 6.84 (d, 1H), 7.24 (d, 1H), 7.26 (s, 1H), 7.38 (dd, 1H), 7.74 (d, 1H), 8.00 (dd, 1H), 8.05 (d, 1H), 8.22 (d, 1H), 12.85 (br s, 1H). MS m/z 417 [M+H]⁺

### Example 2

### 3-ethyl-4-(2-ethyl-4-fluorophenoxy)-N-(1,2,4-thiadiazol-5-yl)benzenesulfonamide diethylamine salt

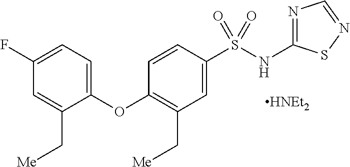 [SMILES: CCc1cc(F)ccc1Oc1ccc(S(=O)(=O)Nc2ncns2)cc1CC ; "·HNEt2"]

A solution of 4-(2-ethyl-4-fluorophenoxy)-3-iodo-N-(1,2,4-thiadiazol-5-yl)benzenesulfonamide (Example 3, 76 mg, 0.15 mmol), diethyl zinc (0.41 mL, 0.45 mmol) and Pd(dppf)Cl₂ (11 mg, 0.015 mmol) in THF (1.5 mL) was heated to 80° C. for 18 hours. The reaction mixture was cooled and quenched by the addition of water (5 mL). The reaction mixture was extracted into DCM (5 mL), the organic layer collected, dried over MgSO₄ and concentrated in vacuo. The residue was purified using preparative HPLC to afford the title compound.

MS m/z 815 [2M+H]⁺

### Example 3

### 4-(2-ethyl-4-fluorophenoxy)-3-iodo-N-(1,2,4-thiadiazol-5-yl)benzenesulfonamide

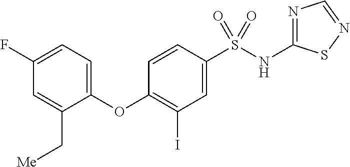 [SMILES: CCc1cc(F)ccc1Oc1ccc(S(=O)(=O)Nc2ncns2)cc1I]

To a suspension of 2-ethyl-4-fluorophenol (491 mg, 3.5 mmol) and KOH (255 mg, 4.55 mmol) in DMSO (18 mL) was added N-(2,4-dimethoxybenzyl)-4-fluoro-3-iodo-N-(1,2,4-thiadiazol-5-yl)benzenesulfonamide (WO2012004706, 2.68 g, 5.00 mmol) and the reaction mixture stirred at room temperature for 6 hours. The reaction was quenched by the addition of saturated aqueous ammonium chloride solution (50 mL) and EtOAc (50 mL). The organic layer was collected, and the aqueous layer washed with EtOAc three times (3×50 mL). The organic layers were combined, dried over MgSO₄ and concentrated in vacuo. The residue was purified using silica gel column chromatography eluting with 0-50% EtOAc in heptanes to afford a colourless gum. The gum was dissolved in DCM (14 mL) and TFA (3.5 mL) was added dropwise at 0° C. The reaction mixture was stirred at room temperature for 2 hours, then quenched with MeOH (50 mL). The resulting precipitate was filtered. The filtrate was concentrated to low volume to afford a suspension. The solid was filtered and recrystallised from EtOAc/Heptane to afford the title compound (1.03 g, 58%).

¹H NMR (400 MHz, DMSO-d₆): δ ppm 1.12 (t, 3H), 2.43-2.51 (m, 2H), 6.69 (d, 1H), 7.05-7.15 (m, 2H), 7.27 (dd, 1H), 7.75 (dd, 1H), 8.20 (d, 1H), 8.48 (s, 1H).

MS m/z 506 [M+H]⁺

### Example 4

### 4-(3-chloro-4-cyanophenoxy)-3-cyano-N-(1-methyl-1H-pyrazol-3-yl)benzenesulfonamide diethylamine salt

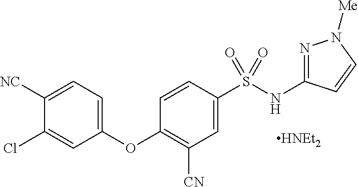 [SMILES: Cn1ccc(NS(=O)(=O)c2ccc(Oc3ccc(C#N)c(Cl)c3)c(C#N)c2)n1 ; "·HNEt2"]

To a solution of 1-methyl-1H-pyrazol-3-amine (55 mg, 0.57 mmol) in DCM (2 mL) was added pyridine (0.1 mL) followed by 4-(3-chloro-4-cyanophenoxy)-3-cyanobenzene-1-sulfonyl chloride (Preparation 3, 100 mg, 0.28 mmol) and the reaction mixture was stirred at room temperature for 18 hours. The reaction was diluted with DCM (10 mL) and washed with 1N HCl (aq) (10 mL) and brine (10 mL). The organic layer was collected, concentrated in vacuo and purified using preparative HPLC to afford the title compound.

MS m/z 412 [M−H]⁻

### Example 5

### 4-(3-chloro-4-cyanophenoxy)-3-cyano-N-(5-methyl-1,2-oxazol-3-yl)benzenesulfonamide diethylamine salt

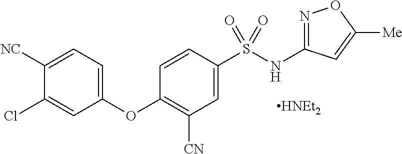 [SMILES: Cc1cc(NS(=O)(=O)c2ccc(Oc3ccc(C#N)c(Cl)c3)c(C#N)c2)no1 ; "·HNEt2"]

The title compound was prepared according to the method described for Example 4 using 5-methylisoxazol-3-amine and isolated.

MS m/z 415 [M+H]⁺

### Example 6

### 4-[2,4-bis(trifluoromethyl)phenoxy]-3-fluoro-N-(1,3-thiazol-2-yl)benzenesulfonamide

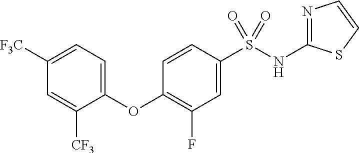 [SMILES: O=S(=O)(Nc1nccs1)c1ccc(Oc2ccc(C(F)(F)F)cc2C(F)(F)F)c(F)c1]

To a cooled (0° C.) solution of 4-[2,4-bis(trifluoromethyl)phenoxy]-3-fluorobenzenesulfonyl chloride (Preparation 6, 0.14 g, 0.33 mmol) in pyridine (3 mL) was added 2-aminothiazole (0.066 g, 0.66 mmol). The reaction mixture was stirred at room temperature for 32 hours before concentrating in vacuo. The residue was dissolved in EtOAc (100 mL) and washed with 1N HCl (aq) (2×25 mL), brine (25 mL), dried over sodium sulfate and concentrated in vacuo. The residue was purified using silica gel column chromatography eluting with 40% EtOAc in heptane to afford the title compound (30 mg, 21%).

MS m/z 487 [M+H]⁺

### Example 7

### 3-cyano-4-[2-fluoro-4-(hydroxymethyl)phenoxy]-N-(1,3-thiazol-2-yl)benzenesulfonamide, diethylamine salt

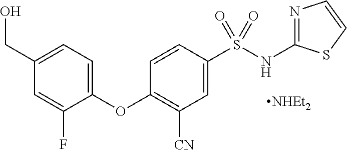 [SMILES: N#Cc1cc(S(=O)(=O)Nc2nccs2)ccc1Oc1ccc(CO)cc1F ; "·NHEt2"]

To a solution of 3-cyano-4-(2-fluoro-4-formylphenoxy)-N-(1,3-thiazol-2-yl)benzenesulfonamide (Preparation 1, 104 mg, 0.258 mmol) in MeOH/DCM (3 mL/3 mL) was added sodium borohydride (9.8 mg, 0.26 mmol) and the reaction mixture allowed to stir at room temperature for 3 hours. The reaction was quenched by the addition of 1M HCl (aq) (5 mL) and extracted into EtOAc (3×25 mL). The organic extracts were combined and concentrated in vacuo. The residue was dissolved in DMSO (1 mL) and purified using preparative HPLC to afford the title compound.

MS m/z 406 [M+H]⁺

### Reference Example 8

### 4-[4-bromo-2-(hydroxymethyl)phenoxy]-3-cyano-N-(1,3-thiazol-2-yl)benzenesulfonamide

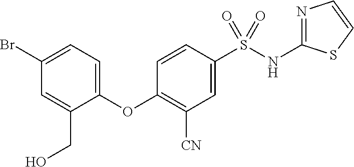 [SMILES: N#Cc1cc(S(=O)(=O)Nc2nccs2)ccc1Oc1ccc(Br)cc1CO]

The title compound was prepared in a manner analogous to that for Example 7, using 3-cyano-4-(4-bromo-4-formylphenoxy)-N-(1,3-thiazol-2-yl)benzenesulfonamide (Preparation 2). The reaction was quenched with saturated aqueous ammonium chloride solution, concentrated in vacuo and the residue taken up in EtOAc and washed with saturated aqueous ammonium chloride solution. The organic layer was dried over MgSO₄ and concentrated in vacuo. The residue was dissolved in DCM (3 mL) and TFA (0.25 mL) was added. The reaction was stirred at room temperature for one hour before concentrating in vacuo and purifying using silica gel column chromatography eluting with 0-10% MeOH in DCM (38 mg, 77%).

MS m/z 465 [M−H]⁻

Unless otherwise specified, the compounds of the Examples that follow were prepared according to the General Method below using one of the Method Variations (MV) described below, followed by one of the Purification Methods (PM) also described below.

### General Method:

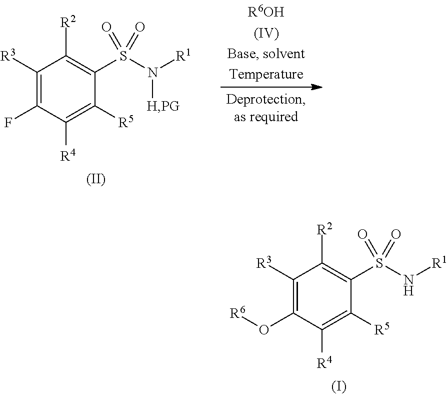

Wherein R¹, R², R³, R⁴, R⁵ and R⁶ are as previously defined for a compound of formula (I), unless otherwise stated, and PG (where present) is a suitable amino protecting group, such as tert-butyl, tert-butyloxycarbonyl, dimethoxybenzyl or allyl.
a) To a solution of a compound of formula (IV) was added an inorganic base (as specifically described in the Method Variations below), followed by a compound of formula (II). The reaction mixture was: cooled, kept at room temperature or heated, as required.
   The reaction mixture was diluted with water, or an aqueous solution of an inorganic acid such as saturated aqueous ammonium chloride or 2N HCl; extracted into a solvent such as DCM or EtOAc; dried over a drying agent such as MgSO₄ or Na₂SO₄; and concentrated in vacuo to afford a residue. Alternatively, the reaction was concentrated in vacuo directly. The residue was purified as necessary.
b) Where required, the residue was deprotected using an acid and/or catalyst to afford the compound of formula (I).

### Method Variations (MV):

### Method 1:

a) Dimethoxybenzyl protected compound (II), K₂CO₃ in DMSO or DMF, at between room temperature to 120° C. for 18 hours.
b) Deprotection with TFA in DCM, or HCl in dioxane, over 48 hours.

### Method 2:

a) Unprotected compound (II), CS₂CO₃ in dioxane at 100° C. for 18 hours.

### Method 3:

a) Unprotected compound (II), K₂CO₃ in DMSO at room temperature for 18 hours.

### Method 4:

a) Unprotected compound (II), K₂CO₃ in DMSO or DMF at 80-100° C. for 18 hours.

### Method 5:

a) Dimethoxybenzyl or tert-butoxycarbonyl protected compound (II), KOH in DMSO, at from 0° C. to room temperature.
b) Deprotection as required with TFA in DCM (when PG is tert-butoxycarbonyl, deprotection occurs under the conditions for effecting the nucleophilic aromatic substitution).

### Method 6:

a) tert-Butoxycarbonyl or tert-butyl protected compound (II), K₂CO₃ in DMF, at from 25-40° C. for between 2-18 hours.
b) Deprotection with TFA or HCl in DCM/dioxane.

### Method 7:

a) tert-Butoxycarbonyl protected compound (II), K₂CO₃ in DMF, at from 25-40° C. for between 2-18 hours.
b) Deprotection occurs under the conditions for effecting the nucleophilic aromatic substitution, or on purification.

### Method 8:

a) Unprotected compound (II), KOH in DMSO, at between 50-140° C. for 18 hours.

### Method 9:

a) Allyl protected compound (II), K₂CO₃ in DMSO or DMF, at 80° C.;
b) Deprotection with palladium tetrakis and barbituric acid.

### Method 10:

a) Unprotected compound (II), NaH in DMF, at room temperature for 18 hours.

### Method 11:

a) tert-Butoxycarbonyl or tert-butyl protected compound (II), NaH in DMF or DMSO, at from room temperature to 100° C. for 18 hours;
b) Deprotection with HCl or TFA in DCM/dioxane.

### Purification Methods (PM):

### Purification Method A:

Silica gel column chromatography eluting with a solvent system selected from:
Between 0-10% MeOH in DCM;
Between 0-100% EtOAc in DCM;
Between 50-100% EtOAc in Heptanes; or
90:10:1 DCM:MeOH:AcOH

### Purification Method B:

### Preparative HPLC

For compounds of the Examples prepared as singletons (i.e. other than via the Library Protocols described hereinafter), one of two preparative HPLC methods was used, as shown below:

### Acidic Conditions

| | |
|---|---|
| Column | Gemini NX C18, 5 um 21.2 × 100 mm |
| Temperature | Ambient |
| Detection | ELSD-MS |
| Mobile Phase A | 0.1% formic acid in water |
| Mobile Phase B | 0.1% formic acid in acetonitrile |

Gradient initial 0% B, 1 mins—5% B; 7 mins—98% B; 9 mins—98% B;
9.1 mins—5% B; 10 mins—5% B

| | |
|---|---|
| Flow rate | 18 mL/min |
| Injection volume | 1000 uL |

Basic conditions

| | |
|---|---|
| Column | Gemini NX C18, 5 um 21.2 × 100 mm |
| Temperature | Ambient |
| Detection | ELSD-MS |
| Mobile Phase A | 0.1% diethylamine in water |
| Mobile Phase B | 0.1% diethylamine in acetonitrile |

Gradient initial 0% B, 1 mins—5% B; 7 mins—98% B; 9 mins—98% B;
9.1 mins—5% B; 10 mins—5% B

| | |
|---|---|
| Flow rate | 18 mL/min |
| Injection volume | 1000 uL |

### Purification Method C:

Trituration with TMBE/ether.

### Purification Method D:

Reverse phase column chromatography using:

Column: Phenomenex Luna C18 5 u 110 A 21.2×150 mm

Detection @ 254 nm, threshold 25 mV

Solvent system:
A: 0.05% formic acid in water, B: 0.05% formic acid in acetonitrile, 0 min 95% A, 2.25 min 95% A, 17.5 min 95% B, 22.5 min 95% B;
Between 5-60% MeCN in water; or
85% A to 100% B over 25 minutes, where mobile phase A is water:MeCN:TFA 7800:200:8 and mobile phase B is MeCN:water:TFA 7200:800:8.

Unless stated otherwise, the compounds of the Examples in the table below were prepared from 3-cyano-4-fluoro-N-(1,2,4-thiadiazol-5-yl)benzenesulfonamide (WO2012004743) or 3-cyano-N-(2,4-dimethoxybenzyl)-4-fluoro-N-1,2,4-thiadiazol-5-yl)benzenesulfonamide (WO2012004743) and the appropriate alcohol of formula (I) according to the specified Method Variation (MV) and, as necessary, purified according to the specified Purification Method (PM).

| | | | Data |
|---|---|---|---|
| Ex | Name | Alcohol | (MV, PM) |
| 9 | 3-cyano-4-(4,5-dichloro-2-methoxyphenoxy)-N-(1,2,4- | 2-methoxy-4,5- | m/z 457 [M + H]⁺ |
| | thiadiazol-5-yl)benzenesulfonamide | dichlorophenol | MV 1 |
| | | | PM A |
| 10 | 4-(5-chloro-2-methoxyphenoxy)-3-cyano-N-(1,2,4- | 2-methoxy-5- | m/z 422 [M + H]⁺ |
| | thiadiazol-5-yl)benzenesulfonamide diethylamine salt | chlorophenol | MV 1 |
| | | | PM B |
| 11 | 4-[5-chloro-2-(propan-2-yloxy)phenoxy]-3-cyano-N-(1,2,4- | 2-(isopropyl)-5- | m/z 451 [M + H]⁺ |
| | thiadiazol-5-yl)benzenesulfonamide diethylamine salt | chlorophenol | MV 1 |
| | | | PM B |
| 12 | 4-(4-chloro-2-ethoxyphenoxy)-3-cyano-N-(1,2,4-thiadiazol- | 2-ethoxy-4- | m/z 435 [M − H]⁻ |
| | 5-yl)benzenesulfonamide | chlorophenol | MV 1, PM B |
| 13 | 4-(4-chlorophenoxy)-3-cyano-N-(1,2,4-thiadiazol-5-yl) | 4-chlorophenol | m/z 393 [M + H]⁺ |
| | benzenesulfonamide diethylamine salt | | MV 2 |
| | | | PM B |
| 14 | 3-cyano-4-[3-(propan-2-yl)phenoxy]-N-(1,2,4-thiadiazol-5- | 3-isopropyl | m/z 401 [M + H]⁺ |
| | yl)benzenesulfonamide diethylamine salt | phenol | MV 4 |
| | | | PM B |
| 15 | 3-cyano-4-(4-cyanophenoxy)-N-(1,2,4-thiadiazol-5-yl) | 4-cyanophenol | m/z 767 |
| | benzenesulfonamide diethylamine salt | | [2M + H]⁺ |
| | | | MV 4, PM B |
| 16 | 3-cyano-4-(4,6-dichlorophenoxy)-N-(1,2,4-thiadiazol-5-yl) | 4,6-dichloro | m/z 427 [M + H]⁺ |
| | benzenesulfonamide | phenol | MV 3, PM C |
| 17 | 3-cyano-4-[(2-ethoxypyridin-3-yl)oxy]-N-(1,2,4-thiadiazol- | 2-ethoxy-3- | m/z 404 [M + H]⁺ |
| | 5-yl)benzenesulfonamide diethylamine salt | hydroxy | MV 1 |
| | | pyridine | PM B |
| 18 | 3-cyano-4-[(4-methoxypyridin-3-yl)oxy]-N-(1,2,4-thiadiazol- | 4-methoxy-3- | m/z 390 [M + H]⁺ |
| | 5-yl)benzenesulfonamide | hydroxy | MV 1 |
| | | pyridine | PM B |
| 19 | 4-[(5-chloropyridin-3-yl)oxy]-3-cyano-N-(1,2,4-thiadiazol- | 5-chloro-3- | m/z 394 [M + H]⁺ |
| | 5-yl)benzenesulfonamide | hydroxypyridine | MV 4, PM A |
| 20 | 3-cyano-4-[(6-methoxypyridin-3-yl)oxy]-N-(1,2,4-thiadiazol- | 6-methoxy-3- | m/z 390 [M + H]⁺ |
| | 5-yl)benzenesulfonamide | hydroxypyridine | MV 4, PM A |
| 21 | 3-cyano-N-(1,2,4-thiadiazol-5-yl)-4-{[6-(trifluoromethyl) | 6-trifluoro | m/z 428 [M + H]⁺ |
| | pyridin-3-yl]oxy}benzenesulfonamide | methyl-3- | MV 4 |
| | | hydroxypyridine | PM A |
| 22 | 4-(4-chloro-2-cyanophenoxy)-3-cyano-N-(1,2,4-thiadiazol-5- | 4-chloro-2- | m/z 418 [M + H]⁺ |
| | yl)benzenesulfonamide | cyanophenol | MV 1, PM D |
| 23 | 3-cyano-4-[4-cyano-2-(difluoromethoxy)phenoxy]-N-(1,2,4- | 4-cyano-2- | m/z 448 [M − H]⁻ |
| | thiadiazol-5-yl)benzenesulfonamide | (difluoro | MV 1 |
| | | methoxy)phenol | PM B |
| 24 | 3-cyano-4-[5-cyano-2-(difluoromethoxy)phenoxy]-N-(1,2,4- | 5-cyano-2- | m/z 899 |
| | thiadiazol-5-yl)benzenesulfonamide diethylamine salt | (difluoro | [2M + H]⁺ |
| | | methoxy)phenol | MV 1 |
| | | | PM B |
| 25 | 3-cyano-4-[2-methoxy-4-(trifluoromethyl)phenoxy]-N- | 2-methoxy-4- | m/z 455 [M − H]⁻ |
| | (1,2,4-thiadiazol-5-yl)benzenesulfonamide diethylamine | (trifluoromethyl) | MV 3 |
| | salt | phenol | PM B |
| 26 | 3-cyano-4-(2,4-dichloro-6-methoxyphenoxy)-N-(1,2,4- | 2,4-dichloro-6- | m/z 457 [M + H]⁺ |
| | thiadiazol-5-yl)benzenesulfonamide diethylamine salt | methoxyphenol | MV 4 |
| | | | PM B |
| 27 | 3-cyano-4-[2-methoxy-4-(trifluoromethoxy)phenoxy]-N- | 2-methoxy-4- | m/z 473 [M + H]⁺ |
| | (1,2,4-thiadiazol-5-yl)benzenesulfonamide diethylamine | (trifluoro | MV 3 |
| | salt | methoxy)phenol | PM B |
| 28 | 3-cyano-4-[2-methoxy-5-(trifluoromethoxy)phenoxy]-N- | 2-methoxy-5- | m/z 473 [M + H]⁺ |
| | (1,2,4-thiadiazol-5-yl)benzenesulfonamide diethylamine | (trifluoro | MV 3 |
| | salt | methoxy)phenol | PM B |
| 29 | 3-cyano-4-[2-methoxy-5-(trifluoromethyl)phenoxy]-N- | 2-methoxy-5- | m/z 457 [M + H]⁺ |
| | (1,2,4-thiadiazol-5-yl)benzenesulfonamide diethylamine | (trifluoromethyl) | MV 4 |
| | salt | phenol | PM B |
| 30 | 4-[4-chloro-2-(cyclobutyloxy)phenoxy]-3-cyano-N-(1,2,4- | 4-chloro-2- | m/z 461 [M − H]⁻ |
| | thiadiazol-5-yl)benzenesulfonamide diethylamine salt | (cyclobutyloxy) | MV 3, |
| | | phenol | PM B |
| 31 | 4-[5-chloro-2-(difluoromethoxy)phenoxy]-3-cyano-N-(1,2,4- | 5-chloro-2- | m/z 457 [M − H]⁻ |
| | thiadiazol-5-yl)benzenesulfonamide | (difluoro | MV 1, |
| | | methoxy)phenol | PM D |
| 32 | 4-[4-chloro-2-(trifluoromethoxy)phenoxy]-3-cyano-N- | 4-chloro-2- | m/z 477 [M + H]⁺ |
| | (1,2,4-thiadiazol-5-yl)benzenesulfonamide diethylamine | (trifluoromethoxy) | MV 4 |
| | salt | phenol | PM B |
| 33 | 4-[2,4-bis(trifluoromethyl)phenoxy]-3-cyano-N-(1,2,4- | 2,4-bis | m/z 495 [M + H]⁺ |
| | thiadiazol-5-yl)benzenesulfonamide | (trifluoromethyl) | MV 4 |
| | | phenol | PM A |
| 34 | 4-(2-chlorophenoxy)-3-cyano-N-(1,2,4-thiadiazol-5-yl) | 2-chlorophenol | m/z 393 [M + H]⁺ |
| | benzenesulfonamide | | MV 8, PM B |
| 35 | 3-cyano-4-(2-(trifluoromethyl)phenoxy)-N-(1,2,4-thiadiazol- | 2-trifluoromethyl | m/z 427 [M + H]⁺ |
| | 5-yl)benzenesulfonamide | phenol | MV 8, PM B |
| 36 | 3-cyano-4-(2-fluorophenoxy)-N-(1,2,4-thiadiazol-5-yl) | 2-fluorophenol | m/z 377 [M + H]⁺ |
| | benzenesulfonamide | | MV 8, PM B |
| 37 | 3-cyano-4-(4-(trifluoromethyl)phenoxy)-N-(1,2,4-thiadiazol- | 2-trifluoromethyl | m/z 427 [M + H]⁺ |
| | 5-yl)benzenesulfonamide | phenol | MV 8, PM B |
| 38 | 4-(4-tert-butyl-2-chlorophenoxy)-3-cyano-N-(1,2,4- | 4-tert-butyl-2- | m/z 449 [M + H]⁺ |
| | thiadiazol-5-yl)benzenesulfonamide | chlorophenol | MV 4 |
| | | | PM B |
| 39 | 3-cyano-4-(4-fluoro-2-methylphenoxy)-N-(1,2,4-thiadiazol- | 4-fluoro-2- | m/z 391 [M + H]⁺ |
| | 5-yl)benzenesulfonamide | methylphenol | MV 4, PM B |
| 40 | 4-(5-chloro-2-methylphenoxy)-3-cyano-N-(1,2,4-thiadiazol- | 5-chloro-2- | m/z 813 |
| | 5-yl)benzenesulfonamide | methylphenol | [2M + H]⁺ |
| | | | MV 4, PM B |
| 41 | 3-cyano-4-(5-fluoro-2-methylphenoxy)-N-(1,2,4-thiadiazol- | 5-fluoro-2- | m/z 391 [M + H]⁺ |
| | 5-yl)benzenesulfonamide | methylphenol | MV 4, PM B |
| 42 | 4-(4-chloro-2-methylphenoxy)-3-cyano-N-(1,2,4-thiadiazol- | 4-chloro-2- | m/z 407[M + H]⁺ |
| | 5-yl)benzenesulfonamide | methylphenol | MV 4, PM B |
| 43 | 3-cyano-4-(2,4-difluorophenoxy)-N-(1,2,4-thiadiazol-5-yl) | 2,4-difluoro | m/z 395 [M + H]⁺ |
| | benzenesulfonamide | phenol | MV 4, PM B |
| 44 | 3-cyano-4-[2-fluoro-5-(trifluoromethyl)phenoxy]-N-(1,2,4- | 2-fluoro-5- | m/z 445 [M + H]⁺ |
| | thiadiazol-5-yl)benzenesulfonamide | (trifluoromethyl) | MV 4 |
| | | phenol | PM B |
| 45 | 4-[2-chloro-5-(trifluoromethyl)phenoxy]-3-cyano-N-(1,2,4- | 2-chloro-5- | m/z 461 [M + H]⁺ |
| | thiadiazol-5-yl)benzenesulfonamide | (trifluoromethyl) | MV 4 |
| | | phenol | PM B |
| 46 | 4-(2-chloro-4-fluorophenoxy)-3-cyano-N-(1,2,4-thiadiazol- | 2-chloro-4- | m/z 411 [M + H]⁺ |
| | 5-yl)benzenesulfonamide | fluorophenol | MV 4, PM B |
| 47 | 3-cyano-4-(2-cyano-4-fluorophenoxy)-N-(1,2,4-thiadiazol- | 2-cyano-4- | m/z 402 [M + H]⁺ |
| | 5-yl)benzenesulfonamide | fluorophenol | MV 4, PM B |
| 48 | 4-(3-chloro-2-fluorophenoxy)-3-cyano-N-(1,2,4-thiadiazol- | 3-chloro-2- | m/z 411 [M + H]⁺ |
| | 5-yl)benzenesulfonamide | fluorophenol | MV 4, PM B |
| 49 | 3-cyano-4-(2,3-difluorophenoxy)-N-(1,2,4-thiadiazol-5-yl) | 2,3-difluoro | m/z 395 [M + H]⁺ |
| | benzenesulfonamide | phenol | MV 4, PM B |
| 50 | 3-cyano-4-(2,5-difluorophenoxy)-N-(1,2,4-thiadiazol-5-yl) | 2,5-difluoro | m/z 395 [M + H]⁺ |
| | benzenesulfonamide | phenol | MV 4, PM B |
| 51 | 3-cyano-4-(4-cyano-2-fluorophenoxy)-N-(1,2,4-thiadiazol- | 4-cyano-2- | m/z 402 [M + H]⁺ |
| | 5-yl)benzenesulfonamide | fluorophenol | MV 4, PM B |
| 52 | 4-(4-chloro-2-fluorophenoxy)-3-cyano-N-(1,2,4-thiadiazol- | 4-chloro-2- | m/z 411 [M + H]⁺ |
| | 5-yl)benzenesulfonamide | fluorophenol | MV 8, PM B |
| 53 | 3-cyano-4-[2-fluoro-3-(trifluoromethyl)phenoxy]-N-(1,2,4- | 2-fluoro-3- | m/z 445 [M + H]⁺ |
| | thiadiazol-5-yl)benzenesulfonamide | (trifluoromethyl) | MV 4 |
| | | phenol | PM B |
| 54 | 3-cyano-N-(1,2,4-thiadiazol-5-yl)-4-[3-(trifluoromethyl) | 3-(trifluoro | m/z 427 [M + H]⁺ |
| | phenoxy]benzene sulfonamide | methyl)phenol | MV 4 |
| | | | PM B |
| 55 | 3-cyano-4-(2,5-dichlorophenoxy)-N-(1,2,4-thiadiazol-5-yl) | 2,5-dichloro | m/z 427 [M + H]⁺ |
| | benzenesulfonamide | phenol | MV 8, PM B |
| 56 | 2-cyano-4-(2-ethyl-4-fluorophenoxy)-N-(1,2,4-thiadiazol-5- | 2-ethyl-4- | m/z 405 [M + H]⁺ |
| | yl)benzenesulfonamide | fluorophenol | MV 4, PM B |
| 57 | 4-{4-chloro-2-[(D₃-methyloxy]phenoxy}-3-cyano-N-(1,2,4- thiadiazol-5-yl)benzenesulfonamide 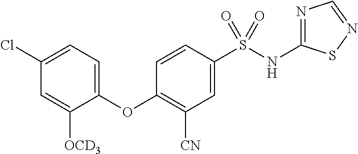 | 4-chloro-2-[(D₃- methyloxy] phenol (Preparation 49) | m/z 426 [M + H]⁺ MV 1 PM B |

The compounds of the Examples in the table below were prepared from appropriate compounds of formulae (II) and (IV) according to the specified Method Variation (MV) and, as necessary, purified according to the specified PM (PM).

| | | | Data |
|---|---|---|---|
| Ex | Name | Sulphonamide and Phenol | MV & PM |
| 58 | 4-(3-chloro-4- | 3-cyano-4-fluoro-N-(1- | m/z 412 |
| | cyanophenoxy)-3-cyano- | methyl-1H-pyrazol-4-yl) | [M − H]− |
| | N-(1-methyl-1H-pyrazol- | benzenesulfonamide and 3- | MV 4 |
| | 4-yl)benzenesulfonamide | chloro-4-cyanophenol | PM D |
| 59 | 4-(3-chloro-4- | 3-cyano-4-fluoro-N-(3- | m/z 413 |
| | cyanophenoxy)-3-cyano- | methyl-1,2-oxazol-5-yl) | [M − H]⁻ |
| | N-(3-methyl-1,2-oxazol- | benzenesulfonamide and 3- | MV 4 |
| | 5-yl)benzenesulfonamide | chloro-4-cyanophenol | PM D |
| 60 | 4-[4-chloro-2- | N-(2,4-dimethoxybenzyl)- | m/z 487 |
| | (trifluoromethoxy) | 3,4-difluoro-N-(5-fluoro- | [M + H]⁺ |
| | phenoxy]-3-fluoro-N- | 1,3-thiazol-2-yl) | MV 1 |
| | (5-fluoro-1,3-thiazol-2-yl) | benzenesulfonamide and 4- | PM A |
| | benzenesulfonamide | chloro-2-(trifluoromethoxy) | |
| | | phenol | |
| 61 | 4-[4-chloro-2- | N-(2,4-dimethoxybenzyl)- | m/z 469 |
| | (trifluoromethyl) | 3,4-difluoro-N-(5-fluoro- | [M − H]⁻ |
| | phenoxy]-3-fluoro-N- | 1,3-thiazol-2-yl) | MV 1 |
| | (5-fluoro-1,3-thiazol-2-yl) | benzenesulfonamide and 4- | PM B |
| | benzenesulfonamide | chloro-2-(trifluoromethyl) | |
| | | phenol | |
| 62 | 3-chloro-4-(4- | 3-chloro-4-fluoro-N-(1,2,4- | m/z 803 |
| | chlorophenoxy)-N- | thiadiazol-5-yl) | [2M + H]⁺ |
| | (1,2,4-thiadiazol-5-yl) | benzenesulfonamide and 2- | MV 4PM |
| | benzenesulfonamide | chlorophenol | |
| | diethylamine salt | | |
| 63 | 3-chloro-4-[3-(propan- | 3-chloro-4-fluoro-N-(1,2,4- | m/z 410 |
| | 2-yl)phenoxy]-N- | thiadiazol-5-yl) | [M + H]⁺ |
| | (1,2,4-thiadiazol-5-yl) | benzenesulfonamide and 3- | MV 4 |
| | benzenesulfonamide | (propan-2-yl)phenol | PM B |
| | diethylamine salt | | |
| 64 | 3-chloro-4-(4- | 3-chloro-4-fluoro-N-(1,2,4- | m/z 785 |
| | cyanophenoxy)-N- | thiadiazol-5-yl) | [2M + H]⁺ |
| | (1,2,4-thiadiazol-5-yl) | benzenesulfonamide and 4- | MV 4 |
| | benzenesulfonamide | cyanophenol | PM B |
| | diethylamine salt | | |
| 65 | 3-chloro-4-[2-fluoro-3- | 3-chloro-4-fluoro-N-(1,2,4- | m/z 454 |
| | (trifluoromethyl) | thiadiazol-5-yl) | [M + H]⁺ |
| | phenoxy]-N-(1,2,4- | benzenesulfonamide and 2- | MV 4 |
| | thiadiazol-5-yl) | fluoro-3-(trifluoromethyl) | PM B |
| | benzenesulfonamide | | |
| | diethylamine salt | | |
| 66 | 3-fluoro-4-[3-(propan- | 3,4-difluoro-N-(prop-2-en- | m/z 393 |
| | 2-yl)phenoxy]-N-(1,3- | 1-yl)-N-(1,3-thiazol-2-yl) | [M + H]⁺ |
| | thiazol-2-yl) | benzenesulfonamide and 3- | MV 9 |
| | benzenesulfonamide | (propan-2-yl)phenol | PM A |
| 67 | 2,5-difluoro-4-[3- | 2,4,5-trifluoro-N-(prop-2- | m/z 411 |
| | (propan-2-yl)phenoxy]- | en-1-yl)-N-(1,3-thiazol-2- | [M + H]⁺ |
| | N-(1,3-thiazol-2-yl) | yl)benzenesulfonamide and | MV 9 |
| | benzenesulfonamide | 3-(propan-2-yl)phenol | PM A |
| 68 | 2,5-difluoro-N-(5-fluoro- | 2,4,5-trifluoro-N-(5-fluoro- | m/z 427 |
| | 1,3-thiazol-2-yl)-4-[3- | 1,3-thiazol-2-yl)-N-(prop- | [M − H]⁻ |
| | (propan-2-yl)phenoxy] | 2-en-1-yl) | MV 9 |
| | benzenesulfonamide | benzenesulfonamide and | PM A |
| | | 3-(propan-2-yl)phenol | |
| 69 | 3-fluoro-N-(5-fluoro- | 4,5-trifluoro-N-(5-fluoro- | m/z 409 |
| | 1,3-thiazol-2-yl)-4-[3- | 1,3-thiazol-2-yl)-N-(prop- | [M − H]⁻ |
| | (propan-2-yl)phenoxy] | 2-en-1-yl) | MV 9 |
| | benzenesulfonamide | benzenesulfonamide and | PM A |
| | | 3-(propan-2-yl)phenol | |
| 70 | 4-[4-chloro-2- | N-(2,4-dimethoxybenzyl)- | m/z 469 |
| | (trifluoromethoxy) | 3,4-difluoro-N-(1,3-thiazol- | [M + H]⁺ |
| | phenoxy]-3-fluoro-N- | 2-yl)benzenesulfonamide | MV 1 |
| | (1,3-thiazol-2-yl) | and 4-chloro-2- | PM B |
| | benzenesulfonamide | (trifluoromethoxy)phenol | |
| 71 | 4-[4-chloro-2- | N-(2,4-dimethoxybenzyl)- | m/z 453 |
| | (trifluoromethyl) | 3,4-difluoro-N-(1,3-thiazol- | [M + H]⁺ |
| | phenoxy]-3-fluoro-N- | 2-yl)benzenesulfonamide | MV 1 |
| | (1,3-thiazol-2-yl) | and 4-chloro-2- | PM B |
| | benzenesulfonamide | (trifluoromethyl)phenol | |
| 72 | 4-(2-ethyl-4- | 2,4-difluoro-N-(5-fluoro- | m/z 397 |
| | fluorophenoxy)-2- | 1,3-thiazol-2-yl) | [M + H]⁺ |
| | fluoro-N-(1,3-thiazol- | benzenesulfonamide and 2- | MV 6 |
| | 2-yl)benzenesulfonamide | ethyl-4-fluorophenol | PM D |
| 73 | 2-fluoro-4-[3-(propan- | N-tert-butyl-2,4-difluoro- | m/z 393 |
| | 2-yl)phenoxy]-N-(1,3- | (N-thiazol-2-yl) | [M + H]⁺ |
| | thiazol-2-yl) | benzenesulfonamide | MV 6 |
| | benzenesulfonamide | (WO2012079443) and 3- | PM D |
| | | (propan-2-yl)phenol | |
| 74 | 3-fluoro-4-(4-fluoro-3- | tert-butyl [(3,4- | m/z 383 |
| | methylphenoxy)-N- | difluorophenyl)sulfonyl] | [M + H]⁺ |
| | (1,3-thiazol-2-yl) | 1,3-thiazol-2-ylcarbamate | MV 11 |
| | benzenesulfonamide | (WO2010079443) and | PM B |
| | | 4-fluoro-3-methylphenol | |
| 75 | 4-(2-bromophenoxy)-3- | tert-butyl [(3,4- | m/z 429 |
| | fluoro-N-(1,3-thiazol- | difluorophenyl)sulfonyl] | [M + H]⁺ |
| | 2-yl)benzenesulfonamide | 1,3-thiazol-2-ylcarbamate | MV 11 |
| | (Reference Example) | (WO2010079443) and | PM A |
| | | 2-bromophenol | |
| 76 | 4-(3-chloro-4- | 3-cyano-4-fluoro-N-(3- | m/z 413 |
| | cyanophenoxy)-3- | methylisoxazol-4-yl) | [M − H]⁻ |
| | cyano-N-(3-methyl- | benzenesulfonamide and 2- | MV 4 |
| | 1,2-oxazol-4-yl) | chloro-4- | PM A |
| | benzenesulfonamide | hydroxybenzonitrile | |
| 77 | 4-(2-ethyl-4- | tert-butyl-(4-fluoro-3- | m/z 505 |
| | fluorophenoxy)-3-iodo- | iodophenyl)sulfonyl(thiazol- | [M + H]⁺ |
| | N-(1,3-thiazol-4-yl) | 4-yl)carbamate and 2-ethyl- | MV 5 |
| | benzenesulfonamide | 4-fluorophenol | PM A |
| 78 | 5-bromo-4-(2-ethyl-4- | 5-bromo-2,4-difluoro-N- | m/z 477 |
| | fluorophenoxy)-2-fluoro- | (1,3-thiazol-4-yl) | [M⁸¹Br + H]⁺ |
| | N-(1,3-thiazol-4-yl) | benzenesulfonamide and | MV 4 |
| | benzenesulfonamide | 2-ethyl-4-fluorophenol | PM A |
| 79 | 4-(4-chloro-2- | tert-butyl [(4-fluoro-2- | m/z 427 |
| | methoxyphenoxy)-2- | methoxyphenyl)sulfonyl] | [M + H]⁺ |
| | methoxy-N-(1,3-thiazol- | 1,3-thiazol-4-ylcarbamate | MV 6 |
| | 4-yl)benzenesulfonamide | and 2-ethyl-4-fluorophenol | PM B |
| | diethylamine salt | | |
| 80 | 4-(4-chloro-2- | N-(2,4-dimethoxybenzyl)- | m/z 428 |
| | methoxyphenoxy)-2- | 4-fluoro-2-chloro-N-(1,3,4- | [M + H]⁺ |
| | methoxy-N-(1,3,4- | thiadiazol-2-yl) | MV 1 |
| | thiadiazol-2-yl) | benzenesulfonamide and 2- | PM A |
| | benzenesulfonamide | ethyl-4-fluorophenol | |
| 81 | 2-chloro-4-(4-chloro-2- | tert-butyl [(4-fluoro-2- | m/z 431 |
| | methoxyphenoxy)-N- | chloro-phenyl)sulfonyl] | [M +H]⁺ |
| | (1,3-thiazol-4-yl) | 1,3-thiazol-4-ylcarbamate | MV 7 |
| | benzenesulfonamide | and 4-chloro-2- | PMs D |
| | diethylamine salt | methoxyphenol | then B |
| 82 | 2-chloro-4-(4-chloro-2- | N-(2,4-dimethoxybenzyl)- | m/z 432 |
| | methoxyphenoxy)-N- | 4-fluoro-2-chloro-N-(1,3,4- | [M + H]⁺ |
| | (1,3,4-thiadiazol-2-yl) | thiadiazol-2-yl) | MV 1 |
| | benzenesulfonamide | benzenesulfonamide and 4- | PM B |
| | diethylamine salt | chloro-2-methoxyphenol | |

### Reference Example 83

### 4-[(5-bromopyrimidin-4-yl)oxy]-3-fluoro-N-(1,3-thiazol-2-yl)benzenesulfonamide

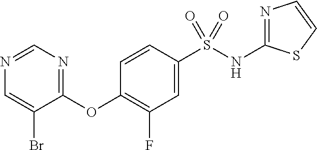 [SMILES: O=S(=O)(Nc1nccs1)c1ccc(Oc2ncncc2Br)c(F)c1]

The title compound was prepared from 5-bromo-2-chloropyrimidine and tert-butyl-3-fluoro-4-hydroxy-N-(thiazol-2-yl)benzenesulfonamide (Preparation 39) according to Method 11 and Purification Method A.

MS m/z 431 [M+H]⁺

The compounds of the Examples in the table below were prepared from N-(5-chloro-1,3-thiazol-2-yl)-3-cyano-4-fluorobenzenesulfonamide (WO2010079443) and the appropriate phenol according to MV 5 and purified according to PM B.

| Ex | Name | Phenol | Data |
|---|---|---|---|
| 84 | N-(5-chloro-1,3-thiazol-2-yl)-3- | 2-fluorophenol | m/z 410 |
| | cyano-4-(2-fluorophenoxy) | | [M + H]⁺ |
| | benzenesulfonamide | | |
| 85 | 4-(2-chlorophenoxy)-N-(5-chloro- | 2-chlorophenol | m/z 426 |
| | 1,3-thiazol-2-yl)-3- | | [M + H]⁺ |
| | cyanobenzenesulfonamide | | |
| 86 | N-(5-chloro-1,3-thiazol-2-yl)-3- | 2,5-dichlorophenol | m/z 460 |
| | cyano-4-(2,5-dichlorophenoxy) | | [M + H]⁺ |
| | benzenesulfonamide | | |
| 87 | 4-(4-chloro-2-fluorophenoxy)-N- | 4-chloro-2- | m/z 444 |
| | (5-chloro-1,3-thiazol-2-yl)-3- | fluorophenol | [M + H]⁺ |
| | cyanobenzenesulfonamide | | |
| 88 | N-(5-chloro-1,3-thiazol-2-yl)-3- | 4-(trifluoromethyl) | m/z 460 |
| | cyano-4-[4-(trifluoromethyl) | phenol | [M + H]⁺ |
| | phenoxy]benzenesulfonamide | | |
| 89 | N-(5-chloro-1,3-thiazol-2-yl)-3- | 3-(trifluoromethyl) | m/z 460 |
| | cyano-4-[3-(trifluoromethyl) | phenol | [M + H]⁺ |
| | phenoxy]benzenesulfonamide | | |
| 90 | N-(5-chloro-1,3-thiazol-2-yl)-3- | 2,4,6- | m/z 482 |
| | cyano-4-(2,4,6-trimethoxyphenoxy) | trimethoxyphenol | [M + H]⁺ |
| | benzenesulfonamide | | |

Unless stated otherwise, the compounds of the Examples in the table below were prepared from 3-cyano-4-fluoro-N-(5-fluoro-1,3-thiazol-2-yl)benzenesulfonamide (WO2012004743) and the appropriate phenol according to MV 4 and purified according to PM B.

| Ex | Name | Phenol | Data |
|---|---|---|---|
| 91 | 4-[4-chloro-2- | 4-chloro-2- | m/z 494 |
| | (trifluoromethoxy)phenoxy]- | (trifluoromethoxy) | [M + H]⁺ |
| | 3-cyano-N-(5-fluoro-1,3- | phenol | |
| | thiazol-2-yl) | | |
| | benzenesulfonamide | | |
| 92 | 4-[4-chloro-2-(trifluoromethyl) | 4-chloro-2- | m/z 478 |
| | phenoxy]-3-cyano-N-(5-fluoro- | (trifluoromethyl) | [M + H]⁺ |
| | 1,3-thiazol-2-yl) | phenol | |
| | benzenesulfonamide | | |
| 93 | 3-cyano-4-(4-fluoro-2- | 4-fluoro-2- | m/z 408 |
| | methylphenoxy)-N-(5-fluoro- | methylphenol | [M + H]⁺ |
| | 1,3-thiazol-2-yl) | | |
| | benzenesulfonamide | | |
| 94 | 3-cyano-4-(2,6- | 2,6-difluorophenol | m/z 412 |
| | difluorophenoxy)-N-(5-fluoro- | | [M + H]⁺ |
| | 1,3-thiazol-2-yl) | | |
| | benzenesulfonamide | | |
| 95 | 3-cyano-4-(4-ethylphenoxy)-N- | 4-ethylphenol | m/z 404 |
| | (5-fluoro-1,3-thiazol-2-yl) | | [M + H]⁺ |
| | benzenesulfonamide | | |
| 96 | 4-(2-chlorophenoxy)-3-cyano- | 2-chlorophenol | m/z 410 |
| | N-(5-fluoro-1,3-thiazol-2-yl) | | [M + H]⁺ |
| | benzenesulfonamide | | |
| 97 | 3-cyano-N-(5-fluoro-1,3- | 2-isopropylphenol | m/z 418 |
| | thiazol-2-yl)-4-[2-(propan-2-yl) | | [M + H]⁺ |
| | phenoxy]benzenesulfonamide | | |
| 98 | 4-(4-chlorophenoxy)-3-cyano- | 4-chlorophenol | m/z 410 |
| | N-(5-fluoro-1,3-thiazol-2-yl) | | [M + H]⁺ |
| | benzenesulfonamide | | |
| 99 | 3-cyano-4-(3-ethylphenoxy)-N- | 3-ethylphenol | m/z 404 |
| | (5-fluoro-1,3-thiazol-2-yl) | | [M + H]⁺ |
| | benzenesulfonamide | | |
| 100 | 3-cyano-4-(3-ethoxyphenoxy)- | 3-ethoxyphenol | m/z 420 |
| | N-(5-fluoro-1,3-thiazol-2-yl) | | [M + H]⁺ |
| | benzenesulfonamide | | |
| 101 | 3-cyano-4-(2,4- | 2,4-dimethylphenol | m/z 404 |
| | dimethylphenoxy)-N-(5-fluoro- | | [M + H]⁺ |
| | 1,3-thiazol-2-yl) | | |
| | benzenesulfonamide | | |
| 102 | 3-cyano-4-(2,5- | 2,5-difluorophenol | m/z 412 |
| | difluorophenoxy)-N-(5-fluoro- | | [M + H]⁺ |
| | 1,3-thiazol-2-yl) | | |
| | benzenesulfonamide | | |
| 103 | 3-cyano-N-(5-fluoro-1,3-thiazol- | 4-isopropylphenol | m/z 418 |
| | 2-yl)-4-[4-(propan-2-yl) | | [M + H]⁺ |
| | phenoxy]benzenesulfonamide | | |
| 104 | 3-cyano-4-(2-fluorophenoxy)- | 2-fluorophenol | m/z 394 |
| | N-(5-fluoro-1,3-thiazol-2-yl) | | [M + H]⁺ |
| | benzenesulfonamide | | |
| 105 | 3-cyano-4-(5-fluoro-2- | 5-fluoro-2- | m/z 408 |
| | methylphenoxy)-N-(5-fluoro- | methylphenol | [M + H]⁺ |
| | 1,3-thiazol-2-yl) | | |
| | benzenesulfonamide | | |
| 106 | 3-cyano-4-(3,5- | 3,5-difluorophenol | m/z 412 |
| | difluorophenoxy)-N-(5-fluoro- | | [M + H]⁺ |
| | 1,3-thiazol-2-yl) | | |
| | benzenesulfonamide | | |
| 107 | 3-cyano-N-(5-fluoro-1,3-thiazol- | 3-isopropylphenol | m/z 418 |
| | 2-yl)-4-[3-(propan-2-yl) | | [M + H]⁺ |
| | phenoxy]benzenesulfonamide | | |
| 108 | 3-cyano-4-(2-ethylphenoxy)-N- | 2-ethylphenol | m/z 404 |
| | (5-fluoro-1,3-thiazol-2-yl) | | [M + H]⁺ |
| | benzenesulfonamide | | |
| 109 | 3-cyano-N-(5-fluoro-1,3-thiazol- | 3-methoxyphenol | m/z 406 |
| | 2-yl)-4-(3-methoxyphenoxy) | | [M + H]⁺ |
| | benzenesulfonamide | | |
| 110 | 4-(3-chlorophenoxy)-3-cyano-N- | 3-chlorophenol | m/z 410 |
| | (5-fluoro-1,3-thiazol-2-yl) | | [M + H]⁺ |
| | benzenesulfonamide | | |
| 111 | 4-(3-chloro-4-fluorophenoxy)- | 3-chloro-4- | m/z 428 |
| | 3-cyano-N-(5-fluoro-1,3-thiazol- | fluorophenol | [M + H]⁺ |
| | 2-yl)benzenesulfonamide | | |
| 112 | 3-cyano-N-(5-fluoro-1,3-thiazol- | 2-methylphenol | m/z 390 |
| | 2-yl)-4-(2-methylphenoxy) | | [M + H]⁺ |
| | benzenesulfonamide | | |
| 113 | 3-cyano-N-(5-fluoro-1,3-thiazol- | 2,4,5-trifluorophenol | m/z 430 |
| | 2-yl)-4-(2,4,5-trifluorophenoxy) | | [M + H]⁺ |
| | benzenesulfonamide | | |
| 114 | 3-cyano-4-(2,3- | 2,3-difluorophenol | m/z 412 |
| | difluorophenoxy)-N-(5-fluoro- | | [M + H]⁺ |
| | 1,3-thiazol-2-yl) | | |
| | benzenesulfonamide | | |
| 115 | 3-cyano-N-(5-fluoro-1,3-thiazol- | 2,3,6-trifluorophenol | m/z 430 |
| | 2-yl)-4-(2,3,6-trifluorophenoxy) | | [M + H]⁺ |
| | benzenesulfonamide | | |
| 116 | 4-(4-chloro-2-fluorophenoxy)- | 4-chloro-2- | m/z 428 |
| | 3-cyano-N-(5-fluoro-1,3-thiazol- | fluorophenol | [M + H]⁺ |
| | 2-yl)benzenesulfonamide | | |
| 117 | 4-(4-chloro-2-methylphenoxy)- | 4-chloro-2- | m/z 424 |
| | 3-cyano-N-(5-fluoro-1,3-thiazol- | methylphenol | [M + H]⁺ |
| | 2-yl)benzenesulfonamide | | |
| 118 | 3-cyano-N-(5-fluoro-1,3-thiazol- | 3,4,5-trifluorophenol | m/z 430 |
| | 2-yl)-4-(3,4,5-trifluorophenoxy) | | [M + H]⁺ |
| | benzenesulfonamide | | |
| 119 | 4-(3-chloro-2-fluorophenoxy)-3- | 3-chloro-2- | m/z 428 |
| | cyano-N-(5-fluoro-1,3-thiazol- | fluorophenol | [M + H]⁺ |
| | 2-yl)benzenesulfonamide | | |
| 120 | 3-cyano-4-(2,3-difluoro-4- | 2,3-difluoro-4- | m/z 426 |
| | methylphenoxy)-N-(5-fluoro-1,3- | methylphenol | [M + H]⁺ |
| | thiazol-2-yl)benzenesulfonamide | | |
| 121 | 3-cyano-N-(5-fluoro-1,3-thiazol- | 2,3,4-trifluorophenol | m/z 430 |
| | 2-yl)-4-(2,3,4-trifluorophenoxy) | | [M + H]⁺ |
| | benzenesulfonamide | | |
| 122 | 3-cyano-4-(3-fluoro-5- | 3-fluoro-5- | m/z 424 |
| | methoxyphenoxy)-N-(5-fluoro- | methoxyphenol | [M + H]⁺ |
| | 1,3-thiazol-2-yl) | | |
| | benzenesulfonamide | | |
| 123 | 3-cyano-4-(2,6-difluoro-3- | 2,6-difluoro-3- | m/z 426 |
| | methylphenoxy)-N-(5-fluoro-1,3- | methylphenol | [M + H]⁺ |
| | thiazol-2-yl)benzenesulfonamide | | |
| 124 | 4-(2-chloro-4-methylphenoxy)-3- | 2-chloro-4- | m/z 424 |
| | cyano-N-(5-fluoro-1,3-thiazol- | methylphenol | [M + H]⁺ |
| | 2-yl)benzenesulfonamide | | |
| 125 | 4-(2-chloro-6-fluorophenoxy)-3- | 2-chloro-6- | m/z 428 |
| | cyano-N-(5-fluoro-1,3-thiazol- | fluorophenol | [M + H]⁺ |
| | 2-yl)benzenesulfonamide | | |
| 126 | 4-(3-chloro-5-fluorophenoxy)-3- | 3-chloro-5- | m/z 426 |
| | cyano-N-(5-fluoro-1,3-thiazol- | fluorophenol | [M − H]⁻ |
| | 2-yl)benzenesulfonamide | | |
| 127 | 4-(2-chloro-6-methylphenoxy)-3- | 2-chloro-6- | m/z 424 |
| | cyano-N-(5-fluoro-1,3-thiazol- | methylphenol | [M + H]⁺ |
| | 2-yl)benzenesulfonamide | | |
| 128 | 3-cyano-4-(3,4-difluoro-2- | 3,4-difluoro-2- | m/z 426 |
| | (methylphenoxy)-N-5-fluoro-1,3- | methylphenol | [M + H]⁺ |
| | thiazol-2-yl)benzenesulfonamide | | |
| 129 | 4-(5-chloro-2-methylphenoxy)-3- | 5-chloro-2- | m/z 424 |
| | cyano-N-(5-fluoro-1,3-thiazol- | methylphenol | [M + H]⁺ |
| | 2-yl)benzenesulfonamide | | |
| 130 | 4-(2-chloro-4-fluorophenoxy)-3- | 2-chloro-4- | m/z 428 |
| | cyano-N-(5-fluoro-1,3-thiazol- | fluorophenol | [M + H]⁺ |
| | 2-yl)benzenesulfonamide | | |
| 131 | 3-cyano-4-(2-ethyl-4- | 2-ethyl-4- | m/z 422 |
| | fluorophenoxy)-N-(5-fluoro- | fluorophenol | [M + H]⁺ |
| | 1,3-thiazol-2-yl) | | |
| | benzenesulfonamide | | |
| 132 | 3-cyano-4-(3-cyanophenoxy)-N- | 3-cyanophenol | m/z 401 |
| | (5-fluoro-1,3-thiazol-2-yl) | | [M + H]⁺ |
| | benzenesulfonamide | | |
| 133 | 3-cyano-N-(5-fluoro-1,3-thiazol- | 3-(trifluoromethyl) | m/z 444 |
| | 2-yl)-4-[3-(trifluoromethyl) | phenol | [M + H]⁺ |
| | phenoxy]benzenesulfonamide | | |
| 134 | 3-cyano-N-(5-fluoro-1,3-thiazol- | 2-fluoro-5- | m/z 462 |
| | 2-yl)-4-[2-fluoro-5- | (trifluoromethyl) | [M + H]⁺ |
| | (trifluoromethyl)phenoxy] | phenol | |
| | benzenesulfonamide | | |
| 135 | 3-cyano-N-(5-fluoro-1,3-thiazol- | 3-(2-hydroxypropan- | m/z 867 |
| | 2-yl)-4-[3-(2-hydroxypropan- | 2-yl)phenol | [2M + H]⁺ |
| | 2-yl)phenoxy] | | |
| | benzenesulfonamide | | |
| 136 | 4-[2-chloro-5-(trifluoromethyl) | 2-chloro-5- | m/z 478 |
| | phenoxy]-3-cyano-N-(5-fluoro- | (trifluoromethyl) | [M + H]⁺ |
| | 1,3-thiazol-2-yl) | phenol | |
| | benzenesulfonamide | | |
| 137 | 4-(3-tert-butylphenoxy)-3-cyano- | 3-tert-butylphenol | m/z 863 |
| | N-(5-fluoro-1,3-thiazol-2-yl) | | [2M + H]⁺ |
| | benzenesulfonamide | | |
| 138 | 4-[3,5-bis(trifluoromethyl) | 3,5-bis | m/z 512 |
| | phenoxy]-3-cyano-N-(5-fluoro- | (trifluoromethyl) | [M + H]⁺ |
| | 1,3-thiazol-2-yl) | phenol | |
| | benzenesulfonamide | | |
| 139 | 3-cyano-N-(5-fluoro-1,3-thiazol- | 3-fluoro-5- | m/z 462 |
| | 2-yl)-4-[3-fluoro-5- | (trifluoromethyl) | [M + H]⁺ |
| | (trifluoromethyl)phenoxy] | phenol | |
| | benzenesulfonamide | | |
| 140 | 3-cyano-4-[4-cyano-3- | 4-cyano-3- | m/z 469 |
| | (trifluoromethyl)phenoxy]-N- | (trifluoromethyl) | [M + H]⁺ |
| | (5-fluoro-1,3-thiazol-2-yl) | phenol | |
| | benzenesulfonamide | | |
| 141 | 4-[4-chloro-3-(trifluoromethyl) | 4-chloro-3- | m/z 478 |
| | phenoxy]-3-cyano-N-(5-fluoro- | (trifluoromethyl) | [M + H]⁺ |
| | 1,3-thiazol-2-yl) | phenol | |
| | benzenesulfonamide | | |
| 142 | 4-(4-chloro-3-ethylphenoxy)-3- | 4-chloro-3- | m/z 438 |
| | cyano-N-(5-fluoro-1,3-thiazol- | ethylphenol | [M + H]⁺ |
| | 2-yl)benzenesulfonamide | | |
| 143 | 3-cyano-N-(5-fluoro-1,3-thiazol- | 4-fluoro-3- | m/z 462 |
| | 2-yl)-4-[4-fluoro-3- | (trifluoromethyl) | [M + H]⁺ |
| | (trifluoromethyl)phenoxy] | phenol | |
| | benzenesulfonamide | | |
| 144 | 4-(2-chloro-5-ethylphenoxy)-3- | 2-chloro-5- | m/z 438 |
| | cyano-N-(5-fluoro-1,3-thiazol- | ethylphenol | [M + H]⁺ |
| | 2-yl)benzenesulfonamide | | |
| | diethylamine salt | | |
| 145 | 3-cyano-N-(5-fluoro-1,3-thiazol- | 4-(2-hydroxyethyl)- | m/z 544 |
| | 2-yl)-4-[4-(2-hydroxyethyl)-2- | 2-iodophenol | [M − H]⁻ |
| | iodophenoxy]benzene- | | PM A. |
| | sulfonamide (Reference Example) | | |
| 146 | 3-cyano-N-(5-fluoro-1,3-thiazol- | 4-(2-hydroxyethyl) | m/z 420 |
| | 2-yl)-4-[4-(2-hydroxyethyl) | phenol | [M + H]⁺ |
| | phenoxy]benzenesulfonamide | | |
| 147 | 3-cyano-N-(5-fluoro-1,3-thiazol- | 4-(3-hydroxypropyl) | m/z 434 |
| | 2-yl)-4-[4-(3-hydroxypropyl) | phenol | [M + H]+ |
| | phenoxy]benzenesulfonamide | | |

The compound of the Example in the table below was prepared from tert-butyl [(3-cyano-4-fluorophenyl)sulfonyl]1,3-thiazol-4-ylcarbamate (WO2010079443) and the appropriate phenol according to MV 6 and purified according to PM D.

| Ex | Name | Phenol | MS Data |
|---|---|---|---|
| 148 | 4-[4-chloro-2-(difluoromethoxy) | 4-chloro-2- | m/z 458 |
| | phenoxy]-3-cyano-N-(1,3-thiazol- | (difluoromethoxy)- | [M + H]⁺ |
| | 4-yl)benzenesulfonamide | phenol | MV 6, PM D |

The compounds of the Examples in the table below were prepared from appropriate compounds of formulae (II) and (IV) according to the specified MV and, as necessary, purified according to the specified PM.

| | | | Data |
|---|---|---|---|
| Ex | Name | Sulphonamide and Phenol | MV & PM |
| 149 | 5-chloro-2-fluoro-4- | tert-Butyl [(5-chloro-2,4- | m/z 527 |
| | (4-fluoro-2- | difluorophenyl)sulfonyl] | [M − H]⁻ |
| | iodophenoxy)- | 1,3-thiazol-4-ylcarbamate | MV 7 |
| | N-(1,3-thiazol-4-yl) | (WO2012004706) and | PM A |
| | benzenesulfonamide | 4-fluoro-2-iodophenol | |
| 150 | 5-chloro-4-(4-chloro-2- | 5-Chloro-N-(2,4- | m/z 450 |
| | methoxyphenoxy)-2- | dimethoxybenzyl)-2,4- | [M + H]⁺ |
| | fluoro-N-(1,3,4- | difluoro-N-1,3,4-thiadiazol- | MV 1 |
| | thiadiazol-2-yl) | 2-ylbenzenesulfonamide | PM A |
| | benzenesulfonamide | (WO2012004743) and | |
| | | 4-chloro-2-methoxyphenol | |
| 151 | 5-chloro-4-[4-chloro-2- | 5-chloro-N-(2,4- | m/z 504 |
| | (trifluoromethoxy) | dimethoxybenzyl)-2,4- | [M + H]⁺ |
| | phenoxy]-2-fluoro-N- | difluoro-N-1,2,4-thiadiazol- | MV 1 |
| | (1,2,4-thiadiazol-5-yl) | 5-ylbenzenesulfonamide | PM A |
| | benzenesulfonamide | (WO2010079443) and | |
| | | 4-chloro-2- | |
| | | trifluoromethoxyphenol | |
| 152 | 5-chloro-4-[4-chloro-2- | 5-Chloro-N-(2,4- | m/z 486 |
| | (difluoromethoxy) | dimethoxybenzyl)-2,4- | [M + H]⁺ |
| | phenoxy]-2-fluoro-N- | difluoro-N-1,3,4- | MV 1 |
| | (1,3,4-thiadiazol-2-yl) | thiadiazol-2- | PM A |
| | benzenesulfonamide | ylbenzenesulfonamide | |
| | | (WO2012004743) and | |
| | | 4-chloro-2- | |
| | | (difluoromethoxy)phenol | |
| 153 | 5-chloro-2-fluoro-4-[2- | 5-chloro-N-(2,4- | m/z 484 |
| | methoxy-4- | dimethoxybenzyl)-2,4- | [M + H]⁺ |
| | (trifluoromethyl) | difluoro-N-1,2,4-thiadiazol- | MV 1 |
| | phenoxy]-N-(1,2,4- | 5-ylbenzenesulfonamide | PM B |
| | thiadiazol-5-yl) | (WO2010079443) and 2- | |
| | benzenesulfonamide | methoxy-4- | |
| | diethylamine salt | (trifluoromethyl)phenol | |
| 154 | 5-chloro-4-(4-chloro-2- | 5-chloro-N-(2,4- | m/z 450 |
| | methoxyphenoxy)-2- | dimethoxybenzyl)-2,4- | [M + H]⁺ |
| | fluoro-N-(1,2,4- | difluoro-N-1,2,4-thiadiazol- | MV 1 |
| | thiadiazol-5-yl) | 5-ylbenzenesulfonamide | PM B |
| | benzenesulfonamide | (WO2010079443) and | |
| | | 4-chloro-2-methoxyphenol | |
| 155 | 5-chloro-4-[4-cyano-2- | 5-chloro-N-(2,4- | m/z 477 |
| | (difluoromethoxy) | dimethoxybenzyl)-2,4- | [M + H]⁺ |
| | phenoxy]-2-fluoro-N- | difluoro-N-1,2,4-thiadiazol- | MV 1 |
| | (1,2,4-thiadiazol-5-yl) | 5-ylbenzenesulfonamide | PM B |
| | benzenesulfonamide | (WO2010079443) and | |
| | diethylamine salt | 4-cyano-2- | |
| | | (difluoromethoxy)phenol | |
| 156 | 5-chloro-4-[5-cyano-2- | 5-chloro-N-(2,4- | m/z 475 |
| | (difluoromethoxy) | dimethoxybenzyl)-2,4- | [M − H]⁻ |
| | phenoxy]-2-fluoro-N- | difluoro-N-1,2,4-thiadiazol- | MV 1 |
| | (1,2,4-thiadiazol-5-yl) | 5-ylbenzenesulfonamide | PM B |
| | benzenesulfonamide | (WO2010079443) and | |
| | diethylamine salt | 5-cyano-2- | |
| | | (difluoromethoxy)phenol | |
| 157 | 5-chloro-4-(2,4-dichloro- | 5-Chloro-N-(2,4- | m/z 484 |
| | 6-methoxyphenoxy)-2- | dimethoxybenzyl)-2,4- | [M + H]⁺ |
| | fluoro-N-(1,3,4- | difluoro-N-1,3,4-thiadiazol- | MV 1 |
| | thiadiazol-2-yl) | 2-ylbenzenesulfonamide | PM B |
| | benzenesulfonamide | (WO2012004743) and | |
| | diethylamine salt | 2,4-dichloro-6- | |
| | | methoxyphenol | |
| 158 | 5-chloro-4-[5-chloro-2- | tert-Butyl [(5-chloro-2,4- | m/z 485 |
| | (difluoromethoxy) | difluorophenyl)sulfonyl] | [M + H]⁺ |
| | phenoxy]-2-fluoro-N- | 1,3-thiazol-4-ylcarbamate | MV 1 |
| | (1,3-thiazol-4-yl) | (WO2012004706) and | PM A |
| | benzenesulfonamide | 5-chloro-2- | |
| | | (difluoromethoxy)phenol | |
| 159 | 5-chloro-4-[4-chloro-2- | tert-Butyl [(5-chloro-2,4- | m/z 485 |
| | (difluoromethoxy) | difluorophenyl)sulfonyl] | [M + H]⁺ |
| | phenoxy]-2-fluoro-N- | 1,3-thiazol-4-ylcarbamate | MV 1 |
| | (1,3-thiazol-4-yl) | (WO2012004706), 4-chloro- | PM D |
| | benzenesulfonamide | 2-(difluoromethoxy)phenol | |
| 160 | 5-chloro-2-fluoro-4-[2- | tert-Butyl [(5-chloro-2,4- | m/z 577 |
| | iodo-5-(trifluoromethyl) | difluorophenyl)sulfonyl] | [M − H]⁻ |
| | phenoxy]-N-(1,3-thiazol- | 1,3-thiazol-4-ylcarbamate | MV 11 |
| | 4-yl)benzene-sulfonamide | (WO2012004706) and | PM A |
| | (Reference Example) | 2-iodo-5-(trifluoromethyl) | |
| | | phenol | |
| 161 | 4-[2-bromo-4- | tert-Butyl [(5-chloro-2,4- | m/z 531 |
| | (trifluoromethyl) | difluorophenyl)sulfonyl] | [M + H]⁺ |
| | phenoxy]-5-chloro-2- | 1,3-thiazol-4-ylcarbamate | MV 11 |
| | fluoro-N-(1,3-thiazol-4- | (WO2012004706) and | PM A |
| | yl)benzenesulfonamide | 2-bromo-4-(trifluoromethyl) | |
| | (Reference Example) | phenol | |

The compounds of the Examples in the table below were prepared from 3-cyano-4-fluoro-N-(1,3-thiazol-2-yl)benzenesulfonamide (WO2012004743) or N-(2,4-dimethoxybenzyl)-3,4-difluoro-N-(1,3-thiazol-2-yl)benzenesulfonamide (WO2012004743) and the appropriate phenol according to the specified MV and, as necessary, purified according to the specified PM.

| | | | Data |
|---|---|---|---|
| Ex | Name | Phenol | MV & PM |
| 162 | 4-[4-chloro-2-(trifluoromethoxy) | 4-chloro-2- | m/z 476 |
| | phenoxy]-3-cyano-N-(1,3- | (trifluoromethoxy) | [M + H]⁺ |
| | thiazol-2-yl)benzenesulfonamide | phenol | MV 4 |
| | | | PM B |
| 163 | 4-[4-chloro-2-(trifluoromethyl) | 4-chloro-2- | m/z 460 |
| | phenoxy]-3-cyano-N-(1,3- | (trifluoromethyl) | [M + H]⁺ |
| | thiazol-2-yl)benzenesulfonamide | phenol | MV 4 |
| | | | PM B |
| 164 | 4-[2-bromo-4-(trifluoromethoxy) | 2-bromo-4- | m/z 522 |
| | phenoxy]-3-cyano-N-(1,3- | (trifluoromethoxy) | [M⁸¹Br + H]⁺ |
| | thiazol-2-yl)benzenesulfonamide | phenol | MV 4, PM A |
| | (Reference Example) | | |
| 165 | 3-cyano-4-(4-iodophenoxy)-N- | 4-iodophenol | m/z 484 |
| | (1,3-thiazol-2-yl) | | [M + H]⁺ |
| | benzenesulfonamide | | MV 4 |
| | (Reference Example) | | |
| 166 | 4-[2-bromo-4-(trifluoromethyl) | 2-bromo-4- | m/z 502 |
| | phenoxy]-3-cyano-N-(1,3- | (trifluoromethyl) | [M − H]⁻ |
| | thiazol-2-yl)benzene-sulfonamide | phenol | MV 4 |
| | (Reference Example) | | PM A |
| 167 | 3-cyano-4-(3-iodophenoxy)-N- | 3-iodophenol | m/z 484 |
| | (1,3-thiazol-2-yl) | | [M + H]⁺ |
| | benzenesulfonamide | | MV 4 |
| | (Reference Example) | | |
| 168 | 4-(4-bromo-3-fluorophenoxy)- | 4-bromo-3- | m/z 454 |
| | 3-cyano-N-(1,3-thiazol-2-yl) | fluorophenol | [M⁸¹Br − H]⁻ |
| | benzenesulfonamide | | MV 4 |
| | (Reference Example) | | |
| 169 | 4-(4-chloro-3-iodophenoxy)-3- | 4-chloro-3- | m/z 518 |
| | cyano-N-(1,3-thiazol-2-yl) | iodophenol | [M + H]⁺ |
| | benzenesulfonamide | | MV 4 |
| | (Reference Example) | | |
| 170 | 4-(4-bromo-2-fluorophenoxy)- | 4-bromo-2- | m/z 454 |
| | 3-cyano-N-(1,3-thiazol-2-yl) | fluorophenol | [M⁸¹Br − H]⁻ |
| | benzenesulfonamide | | MV 4 |
| | (Reference Example) | | |
| 171 | 4-[(6-bromopyridin-3-yl)oxy]- | 2-bromo-5- | m/z 437 |
| | 3-cyano-N-(1,3-thiazol-2-yl) | hydroxypyridine | [M⁸¹Br − H]⁻ |
| | benzenesulfonamide | | MV 4, PM 1 |
| | (Reference Example) | | |
| 172 | 4-[(6-chloropyridin-3-yl)oxy]- | 2-chloro-5- | m/z 393 |
| | 3-cyano-N-(1,3-thiazol-2-yl) | hydroxypyridine | [M + H]⁺ |
| | benzenesulfonamide | | MV 4 |
| 173 | 3-cyano-4-[(6-methoxypyridin- | 2-methoxy-5- | m/z 389 |
| | 3-yl)oxy]-N-(1,3-thiazol-2-yl) | hydroxyphenol | [M + H]⁺ |
| | benzenesulfonamide | | MV 4 |
| 174 | 3-cyano-4-[3-cyano-5-(propan- | 3-cyano-5- | m/z 849 |
| | 2-yl)phenoxy]-N-(1,3-thiazol- | (propan-2-yl) | [2M + H]⁺ |
| | 2-yl)benzenesulfonamide | phenol | MV 2, PM B |
| | diethylamine salt | | |
| 175 | 3-cyano-4-[3-(2-hydroxyethyl) | 3-(2- | m/z 400 |
| | phenoxy]-N-(1,3-thiazol-2-yl) | hydroxyethyl) | [M − H]⁻ |
| | benzenesulfonamide | phenol | MV 4 |
| | | | PM A |
| 176 | 3-cyano-4-[2-(2-hydroxyethyl) | 2-(2- | m/z 400 |
| | phenoxy]-N-(1,3-thiazol-2-yl) | hydroxyethyl) | [M − H]⁻ |
| | benzenesulfonamide | phenol | MV 4 |
| | | | PM A |
| 177 | 3-cyano-4-[3-(hydroxymethyl) | 3-(hydroxymethyl) | m/z 386 |
| | phenoxy]-N-(1,3-thiazol-2-yl) | phenol | [M − H]⁻ |
| | benzenesulfonamide | | MV 4, PM A |
| 178 | 3-cyano-4-(2-fluoro-4- | 2-fluoro-4- | m/z 502 |
| | iodophenoxy)-N-(1,3-thiazol- | iodophenol | [M + H]⁺ |
| | 2-yl)benzenesulfonamide | | MV 4, PM C |
| | (Reference Example) | | |
| 179 | 3-cyano-4-(2-ethyl-4- | 2-ethyl-4- | m/z 404 |
| | fluorophenoxy)-N-1,3-thiazol- | fluorophenol | [M + H]⁺ |
| | 2-yl)benzenesulfonamide | | MV 11, |
| | | | PM C |
| 180 | 4-(3-chloro-2-cyanophenoxy)- | 3-chloro-2- | m/z 417 |
| | 3-cyano-N-(1,3-thiazol-2-yl) | cyanophenol | [M + H]⁺ |
| | benzenesulfonamide | | MV 4, |
| | | | PM D |
| 181 | 4-(2-chlorophenoxy)-3-cyano- | 2-chlorophenol | m/z 392 |
| | N-(1,3-thiazol-2-yl) | | [M + H]⁺ |
| | benzenesulfonamide | | MV 13, |
| | | | PM A |
| 182 | 4-(2-bromophenoxy)-3-cyano- | 2-bromophenol | m/z 434 |
| | N-(1,3-thiazol-2-yl) | | [M − H]⁻ |
| | benzenesulfonamide | | MV 11, |
| | (Reference Example) | | PM A |
| 183 | 3-cyano-4-(2-fluorophenoxy)- | 2-fluorophenol | m/z 376 |
| | N-(1,3-thiazol-2-yl) | | [M + H]⁺ |
| | benzenesulfonamide | | MV 11, |
| | | | PM A |
| 184 | 4-(4-aminophenoxy)-3-cyano- | 4-aminophenol | m/z 371 |
| | N-(1,3-thiazol-2-yl) | | [M − H]⁻ |
| | benzenesulfonamide | | MV 1, |
| | | | PM A |
| 185 | 3-cyano-4-[(6-fluoropyridin-3- | 2-fluoro-5- | m/z 377 |
| | yl)oxy]-N-(1,3-thiazol-2-yl) | hydroxypyridine | [M + H]⁺ |
| | benzenesulfonamide | | MV 4 |

### Example 186

### 3-cyano-4-{[6-(dimethylamino)pyridin-3-yl]oxy}-N-(1,3-thiazol-2-yl)benzenesulfonamide

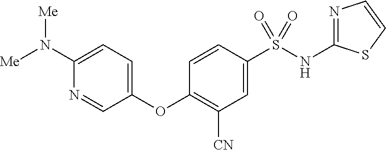 [SMILES: CN(C)c1ccc(Oc2ccc(S(=O)(=O)Nc3nccs3)cc2C#N)cn1]

To a solution of 3-cyano-4-[(6-fluoropyridin-3-yl)oxy]-N-(1,3-thiazol-2-yl)benzenesulfonamide (Example 185, 100 mg, 0.27 mmol) in DMF (1 mL) was added dimethylamine (5 mL) and the reaction mixture heated to 90° C. for 18 hours. The reaction mixture was cooled, concentrated in vacuo and purified using preparative HPLC to afford the title compound.

MS m/z 402 [M+H]⁺

### Example 187

### 3-cyano-4-{[6-(methylamino)pyridin-3-yl]oxy}-N-(1,3-thiazol-2-yl)benzenesulfonamide

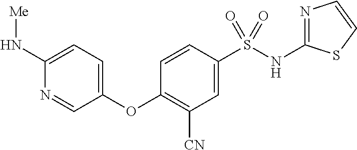 [SMILES: CNc1ccc(Oc2ccc(S(=O)(=O)Nc3nccs3)cc2C#N)cn1]

The title compound was prepared according to the Method described for Example 186 using methylamine.

MS m/z 388 [M+H]⁺

### Example 188

### 3-cyano-4-{[6-(propan-2-ylamino)pyridin-3-yl]oxy}-N-(1,3-thiazol-2-yl)benzenesulfonamide

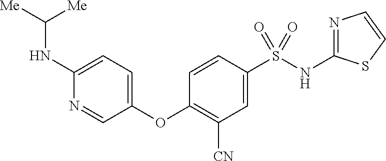 [SMILES: CC(C)Nc1ccc(Oc2ccc(S(=O)(=O)Nc3nccs3)cc2C#N)cn1]

The title compound was prepared according to the Method described for Example 186 using isopropylamine.

MS m/z 416 [M+H]⁺

### Example 189

### 3-cyano-4-{[6-(ethylamino)pyridin-3-yl]oxy}-N-(1,3-thiazol-2-yl)benzenesulfonamide

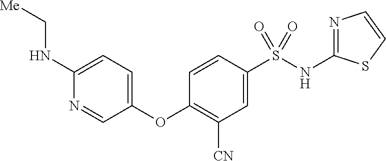 [SMILES: CCNc1ccc(Oc2ccc(S(=O)(=O)Nc3nccs3)cc2C#N)cn1]

The title compound was prepared according to the Method described for Example 186 using ethylamine in THF.

MS m/z 402 [M+H]⁺

### Example 190

### 3-cyano-N-(1,3-thiazol-2-yl)-4-{[6-(2,2,2-trifluoroethoxy)pyridin-3-yl]oxy}benzenesulfonamide

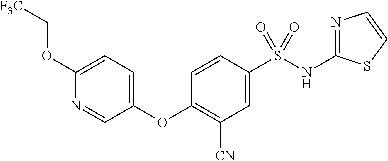 [SMILES: N#Cc1cc(S(=O)(=O)Nc2nccs2)ccc1Oc1ccc(OCC(F)(F)F)nc1]

To trifluoroethanol (0.23 mL, 3.2 mmol) in DMF (5 mL) was added NaH (64 mg, 1.6 mmol) and the reaction was stirred at room temperature for 60 minutes. 3-Cyano-4-[(6-fluoropyridin-3-yl)oxy]-N-(1,3-thiazol-2-yl)benzenesulfonamide (Example 185, 150 mg, 0.38 mmol) was added in portions and the mixture stirred at room temperature for 48 hours. The reaction mixture was poured into aqueous ammonium chloride solution and extracted with EtOAc (3×20 mL). The organic layers were combined, washed with water, dried over MgSO₄ and concentrated in vacuo. The residue was purified using preparative HPLC to afford the title compound.

MS m/z 457 [M+H]⁺

### Example 191

### 3-cyano-4-[(6-ethoxypyridin-3-yl)oxy]-N-(1,3-thiazol-2-yl)benzenesulfonamide

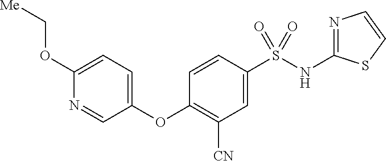 [SMILES: CCOc1ccc(Oc2ccc(S(=O)(=O)Nc3nccs3)cc2C#N)cn1]

The title compound was prepared according to the Method described for Example 190 using ethanol.

MS m/z 403 [M+H]⁺

### Example 192

### 3-cyano-4-{[6-(propan-2-yloxy)pyridin-3-yl]oxy}-N-(1,3-thiazol-2-yl)benzenesulfonamide

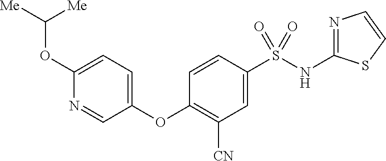 [SMILES: CC(C)Oc1ccc(Oc2ccc(S(=O)(=O)Nc3nccs3)cc2C#N)cn1]

The title compound was prepared according to the Method described for Example 190 at between 50-110° C. using isopropanol.

MS m/z 417 [M+H]⁺

### Library Protocol 1:

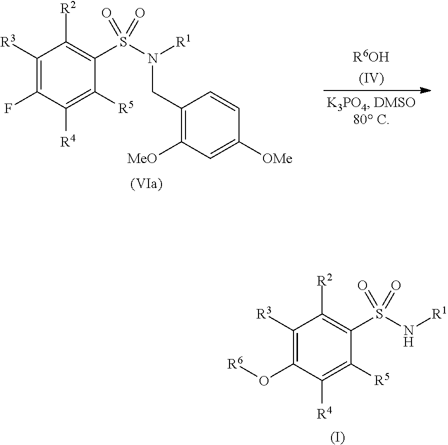

A 0.2M solution in DMSO of the compound of formula (IV) (500 μL, 100 μmol) was added to a 0.2M solution in DMSO of the compound of formula (Via) (500 μL, 100 μmol) followed by anhydrous potassium phosphate (64 mg, 300 μmol). The reaction mixture was heated to 80° C. for 16 hours before concentrating in vacuo. The residue was dissolved in DMSO (1 mL) and purified using preparative HPLC as described below to afford the desired compound of formula (I).

### Preparative HPLC Method 1:

Mobile phase A: 10 mM ammonium acetate in water; Mobile phase B: MeCN

Column: Xterra RP18C18 (250×19 mm×10 u) or Reprosil Gold C18 (20×250 mm, 5 u)

Gradient: Initial 10% B; 3 mins 30% B; 18 mins 60% B, 19 mins 95% B, 22-25 mins 10% B,

Flow rate: 20 mL/min.

### Preparative HPLC Method 2:

Mobile phase A: 0.1% formic acid in water; Mobile phase B: MeCN

Column: Reprosil Gold C18 (20×250 mm, 5 u)

Gradient: Initial 10% B; 3 mins 40% B; 18 mins 70% B, 19 mins 95% B, 22-25 mins 10% B,

Flow rate: 20 mL/min

### LCMS Conditions:

Mobile phase A: 0.05% formic acid in water; Mobile phase B: MeCN

Column: RESTEK C18 2.1×30 mm×3μ

Gradient: From 98% [A] and 2% [B] to 90% [A] and 10% [B] in 1 min, further to 2% [A] and 98% [B] in 2.0 min and finally back to initial condition in 3 min,

Flow rate: 1.5 mL/min

The compounds of the Examples in the table below were prepared from the appropriate sulphonamide and: (a) 3-cyano-4-fluorophenol; (b) 3,4-difluorophenol or (c) 3-chloro-4-cyanophenol; according to Library Protocol 1.

| Ex | Name | Sulfonamide | MS Data |
|---|---|---|---|
| 193 | N-(5-chloro-1,3-thiazol-2-yl)-3- | N-(5-chloro-1,3-thiazol-2-yl)-3- | m/z 435 |
| | cyano-4-(3-cyano-4- | cyano-N-(2,4-dimethoxybenzyl)- | [M + H]⁺ |
| | fluorophenoxy)benzenesulfonamide | 4-fluorobenzenesulfonamide | |
| | | (WO2010079443) | |
| 194 | 4-(3-cyano-4-fluorophenoxy)-3- | N-(2,4-dimethoxybenzyl)-3,4- | m/z 395 |
| | fluoro-N-(1,2,4-thiadiazol-5- | difluoro-N-(1,2,4-thiadiazol-5- | [M + H]⁺ |
| | yl)benzenesulfonamide | yl)benzenesulfonamide | |
| 195 | 4-(3,4-difluorophenoxy)-3- | N-(2,4-dimethoxybenzyl)-3,4- | m/z 388 |
| | fluoro-N-(1,2,4-thiadiazol-5- | difluoro-N-(1,2,4-thiadiazol-5- | [M + H]⁺ |
| | yl)benzenesulfonamide | yl)benzenesulfonamide | |
| 196 | 4-(3,4-difluorophenoxy)-2,5- | N-(2,4-dimethoxybenzyl)-2,4,5- | m/z 406 |
| | difluoro-N-(1,2,4-thiadiazol-5- | trifluoro-N-(1,2,4-thiadiazol-5- | [M + H]⁺ |
| | yl)benzenesulfonamide | yl)benzenesulfonamide | |
| | | (WO2010079443) | |
| 197 | N-(5-chloro-1,3-thiazol-2-yl)-3- | N-(5-chloro-1,3-thiazol-2-yl)-3- | m/z 428 |
| | cyano-4-(3,4- | cyano-N-(2,4-dimethoxybenzyl)- | [M + H]⁺ |
| | difluorophenoxy)benzenesulfonamide | 4-fluorobenzenesulfonamide | |
| | | (WO2010079443) | |
| 198 | 4-(3,4-difluorophenoxy)-3,5- | N-(2,4-dimethoxybenzyl)-3,4,5- | m/z 406 |
| | difluoro-N-(1,2,4-thiadiazol-5- | trifluoro-N-(1,2,4-thiadiazol-5- | [M + H]⁺ |
| | yl)benzenesulfonamide | yl)benzenesulfonamide | |
| 199 | 3-cyano-4-(3-cyano-4- | 3-cyano-N-(2,4- | m/z 402 |
| | fluorophenoxy)-N-(1,3,4- | dimethoxybenzyl)-4-fluoro-N- | [M + H]⁺ |
| | thiadiazol-2- | (1,3,4-thiadiazol-2- | |
| | yl)benzenesulfonamide | yl)benzenesulfonamide | |
| | | (WO2012004743) | |
| 200 | 4-(3,4-difluorophenoxy)-2- | N-(2,4-dimethoxybenzyl)-2,4- | m/z 402 |
| | fluoro-5-methyl-N-(1,3,4- | difluoro-5-methyl-N-(1,3,4- | [M + H]⁺ |
| | thiadiazol-2- | thiadiazol-2- | |
| | yl)benzenesulfonamide | yl)benzenesulfonamide | |
| 201 | 3-cyano-4-(3,4- | 3-cyano-N-(2,4- | m/z 395 |
| | difluorophenoxy)-N-(1,3,4- | dimethoxybenzyl)-4-fluoro-N- | [M + H]⁺ |
| | thiadiazol-2- | (1,3,4-thiadiazol-2- | |
| | yl)benzenesulfonamide | yl)benzenesulfonamide | |
| | | (WO2012004743) | |
| 202 | 3-chloro-4-(3,4- | 3-chloro-N-(2,4- | m/z 404 |
| | difluorophenoxy)-N-(1,2,4- | dimethoxybenzyl)-4-fluoro-N- | [M + H]⁺ |
| | thiadiazol-5- | (1,2,4-thiadiazol-5- | |
| | yl)benzenesulfonamide | yl)benzenesulfonamide | |
| | | (WO2010079443) | |
| 203 | 3-chloro-4-(3-chloro-4- | 3-chloro-N-(2,4- | m/z 427 |
| | cyanophenoxy)-N-(1,3,4- | dimethoxybenzyl)-4-fluoro-N- | [M + H]⁺ |
| | thiadiazol-2- | (1,3,4-thiadiazol-2- | |
| | yl)benzenesulfonamide | yl)benzenesulfonamide | |
| | | (WO2012004743) | |
| 204 | 3-chloro-4-(3,4- | 3-chloro-N-(2,4- | m/z 404 |
| | difluorophenoxy)-N-(1,3,4- | dimethoxybenzyl)-4-fluoro-N- | [M + H]⁺ |
| | thiadiazol-2- | (1,3,4-thiadiazol-2- | |
| | yl)benzenesulfonamide | yl)benzenesulfonamide | |
| | | (WO2012004743) | |
| 205 | 4-(3-cyano-4-fluorophenoxy)- | N-(2,4-dimethoxybenzyl)-3,4,5- | m/z 413 |
| | 3,5-difluoro-N-(1,2,4-thiadiazol- | trifluoro-N-(1,2,4-thiadiazol-5- | [M + H]⁺ |
| | 5-yl)benzenesulfonamide | yl)benzenesulfonamide | |
| 206 | 3-cyano-4-(3-cyano-4- | 3-cyano-N-(2,4- | m/z 402 |
| | fluorophenoxy)-N-(1,2,4- | dimethoxybenzyl)-4-fluoro-N- | [M + H]⁺ |
| | thiadiazol-5- | (1,2,4-thiadiazol-5- | |
| | yl)benzenesulfonamide | yl)benzenesulfonamide | |
| | | (WO2012004743) | |
| 207 | 3-chloro-4-(3-cyano-4- | 3-chloro-N-(2,4- | m/z 409 |
| | fluorophenoxy)-N-(1,2,4- | dimethoxybenzyl)-4-fluoro-N- | [M − H]⁻ |
| | thiadiazol-5- | (1,2,4-thiadiazol-5- | |
| | yl)benzenesulfonamide | yl)benzenesulfonamide | |
| | | (WO2010079443) | |
| 208 | 4-(3-chloro-4-cyanophenoxy)- | N-(2,4-dimethoxybenzyl)-2,4,5- | m/z 428 |
| | 2,5-difluoro-N-(1,2,4-thiadiazol- | trifluoro-N-(1,2,4-thiadiazol-5- | [M + H]⁺ |
| | 5-yl)benzenesulfonamide | yl)benzenesulfonamide | |
| | | (WO2010079443) | |
| 209 | 4-(3,4-difluorophenoxy)-2,5- | N-(2,4-dimethoxybenzyl)-2,4,5- | m/z 406 |
| | difluoro-N-(1,3,4-thiadiazol-2- | trifluoro-N-(1,3,4-thiadiazol-2- | [M + H]⁺ |
| | yl)benzenesulfonamide | yl)benzenesulfonamide | |
| 210 | 3-chloro-4-(3-chloro-4- | 3-Chloro-N-(2,4- | m/z 425 |
| | cyanophenoxy)-N-(1,2,4- | dimethoxybenzyl)-4-fluoro-N- | [M − H]⁻ |
| | thiadiazol-5- | (1,2,4-thiadiazol-5- | |
| | yl)benzenesulfonamide | yl)benzenesulfonamide | |
| | | (WO2010079443) | |
| 211 | 4-(3,4-difluorophenoxy)-3- | N-(2,4-dimethoxybenzyl)-4-fluoro- | m/z 382 |
| | methyl-N-(1,2,4-thiadiazol-5- | 3-methyl-N-(1,2,4-thiadiazol-5- | [M − H]⁻ |
| | yl)benzenesulfonamide | yl)benzenesulfonamide | |
| 212 | 3-cyano-4-(3,4- | 3-cyano-N-(2,4- | m/z 393 |
| | difluorophenoxy)-N-(1,2,4- | dimethoxybenzyl)-4-fluoro-N- | [M − H]⁻ |
| | thiadiazol-5- | (1,2,4-thiadiazol-5- | |
| | yl)benzenesulfonamide | yl)benzenesulfonamide | |
| | | (WO2012004743) | |
| 213 | 4-(3-cyano-4-fluorophenoxy)- | N-(2,4-dimethoxybenzyl)-2,4,5- | m/z 413 |
| | 2,5-difluoro-N-(1,2,4-thiadiazol- | trifluoro-N-(1,2,4-thiadiazol-5- | [M + H]⁺ |
| | 5-yl)benzenesulfonamide | yl)benzenesulfonamide | |
| | | (WO2010079443) | |
| 214 | 4-(3-cyano-4-fluorophenoxy)-2- | N-(2,4-dimethoxybenzyl)-2,4- | m/z 409 |
| | fluoro-5-methyl-N-(1,3,4- | difluoro-5-methyl-N-(1,3,4- | [M + H]⁺ |
| | thiadiazol-2- | thiadiazol-2- | |
| | yl)benzenesulfonamide | yl)benzenesulfonamide | |
| 215 | 4-(3-chloro-4-cyanophenoxy)- | N-(2,4-dimethoxybenzyl)-3,4,5- | m/z 427 |
| | 3,5-difluoro-N-(1,2,4-thiadiazol- | trifluoro-N-(1,2,4-thiadiazol-5- | [M − H]⁻ |
| | 5-yl)benzenesulfonamide | yl)benzenesulfonamide | |
| 216 | 3-chloro-4-(3-cyano-4- | 3-chloro-N-(2,4- | m/z 411 |
| | fluorophenoxy)-N-(1,3,4- | dimethoxybenzyl)-4-fluoro-N- | [M + H]⁺ |
| | thiadiazol-2- | (1,3,4-thiadiazol-2- | |
| | yl)benzenesulfonamide | yl)benzenesulfonamide | |
| | | (WO2012004743) | |
| 217 | 4-(3-cyano-4-fluorophenoxy)- | N-(2,4-dimethoxybenzyl)-2,4,5- | m/z 413 |
| | 2,5-difluoro-N-(1,3,4-thiadiazol- | trifluoro-N-(1,3,4-thiadiazol-2- | [M + H]⁺ |
| | 2-yl)benzenesulfonamide | yl)benzenesulfonamide | |
| 218 | 4-(3-chloro-4-cyanophenoxy)- | 3-cyano-N-(2,4- | m/z 416 |
| | 3-cyano-N-(1,2,4-thiadiazol-5- | dimethoxybenzyl)-4-fluoro-N- | [M − H]⁻ |
| | yl)benzenesulfonamide | (1,2,4-thiadiazol-5- | |
| | | yl)benzenesulfonamide | |
| | | (WO2012004743) | |
| 219 | 4-(3-chloro-4-cyanophenoxy)- | 3-cyano-N-(2,4- | m/z 416 |
| | 3-cyano-N-(1,3,4-thiadiazol-2- | dimethoxybenzyl)-4-fluoro-N- | [M − H]⁻ |
| | yl)benzenesulfonamide | (1,3,4-thiadiazol-2- | |
| | | yl)benzenesulfonamide | |
| | | (WO2012004743) | |

### Library Protocol 2:

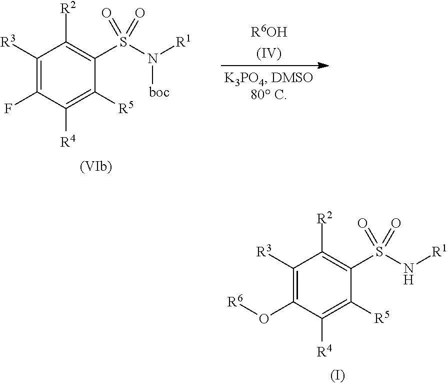

A 0.2M solution in DMSO of the compound of formula (IV) (500 μL, 100 μmol) was added to a 0.2M solution in DMSO of the compound of formula (VIb) (500 μL, 100 μmol) followed by anhydrous potassium phosphate (64 mg, 300 μmol). The reaction mixture was heated to 80° C. for 16 hours before concentrating in vacuo. The residue was dissolved in DMSO (1 mL) and purified using preparative HPLC as described below to afford the desired compound of formula (I).

### Preparative HPLC Method 1:

Mobile phase A: 10 mM ammonium acetate in water; Mobile phase B: MeCN

Column: Gemini NX C18 (21×100 mm, 5 u)

Gradient: Initial 10% B; 2 mins 30% B; 10 mins 60% B, 12 mins 95% B, 14-15 mins 10% B

Flow rate: 20 mL/min

### LCMS Conditions:

Mobile phase A: 0.05% formic acid in water; Mobile phase B: MeCN

Column: RESTEK C18 2.1×30 mm×3μ

Gradient: From 98% [A] and 2% [B] to 90% [A] and 10% [B] in 1 min, further to 2% [A] and 98% [B] in 2.0 min and finally back to initial condition in 3 min

Flow rate: 1.5 mL/min.

The compounds of the Examples in the table below were prepared from the appropriate sulphonamide and: (a) 3-cyano-4-fluorophenol; (b) 3,4-difluorophenol or (c) 3-chloro-4-cyanophenol; according to Library Protocol 2.

| Ex | Name | Sulfonamide | MS Data |
|---|---|---|---|
| 220 | 4-(3-chloro-4-cyanophenoxy)- | tert-Butyl [(3-cyano-4- | m/z 415 |
| | 3-cyano-N-(1,3-thiazol-4- | fluorophenyl)sulfonyl]1,3-thiazol- | [M − H]⁻ |
| | yl)benzenesulfonamide | 4-ylcarbamate (WO2010079443) | |
| 221 | 4-(3-cyano-4-fluorophenoxy)- | tert-Butyl 1,3-thiazol-4-yl[(2,4,5- | m/z 412 |
| | 2,5-difluoro-N-(1,3-thiazol-4- | trifluorophenyl)sulfonyl]carbamate | [M + H]⁺ |
| | yl)benzenesulfonamide | (WO2012004706) | |
| 222 | 5-bromo-4-(3-cyano-4- | tert-butyl [(5-bromo-2,4- | m/z 472 |
| | fluorophenoxy)-2-fluoro-N-(1,3- | difluorophenyl)sulfonyl]1,3- | [M + H]⁺ |
| | thiazol-4- | thiazol-4-ylcarbamate | |
| | yl)benzenesulfonamide | | |
| 223 | 4-(3,4-difluorophenoxy)-2- | tert-butyl [(2,4-difluoro-5- | m/z 401 |
| | fluoro-5-methyl-N-(1,3-thiazol- | methylphenyl)sulfonyl]1,3-thiazol- | [M + H]⁺ |
| | 4-yl)benzenesulfonamide | 4-ylcarbamate | |
| 224 | 4-(3,4-difluorophenoxy)-2- | tert-butyl [(2,4- | m/z 387 |
| | fluoro-N-(1,3-thiazol-4- | difluorophenyl)sulfonyl]1,3- | [M + H]⁺ |
| | yl)benzenesulfonamide | thiazol-4-ylcarbamate | |
| 225 | 4-(3-chloro-4-cyanophenoxy)- | tert-butyl [(2,4- | m/z 410 |
| | 2-fluoro-N-(1,3-thiazol-4- | difluorophenyl)sulfonyl]1,3- | [M + H]⁺ |
| | yl)benzenesulfonamide | thiazol-4-ylcarbamate | |
| 226 | 4-(3,4-difluorophenoxy)-3- | tert-butyl [(3,4- | m/z 387 |
| | fluoro-N-(1,3-thiazol-2- | difluorophenyl)sulfonyl]1,3- | [M + H]⁺ |
| | yl)benzenesulfonamide | thiazol-2-ylcarbamate | |
| | | (WO2010079443)) | |
| 227 | 2-chloro-4-(3-chloro-4- | tert-butyl [(4-fluoro-2-chloro- | m/z 426 |
| | cyanophenoxy)-N-(1,3-thiazol- | phenyl)sulfonyl]1,3-thiazol-4- | [M + H]⁺ |
| | 4-yl)benzenesulfonamide | ylcarbamate | |
| 228 | 3-cyano-4-(3,4- | tert-Butyl [(3-cyano-4- | m/z 394 |
| | difluorophenoxy)-N-(1,3- | fluorophenyl)sulfonyl]1,3-thiazol- | [M + H]⁺ |
| | thiazol-4- | 4-ylcarbamate (WO2010079443) | |
| | yl)benzenesulfonamide | | |
| 229 | 5-chloro-4-(3,4- | tert-Butyl [(5-chloro-2,4- | m/z 421 |
| | difluorophenoxy)-2-fluoro-N- | difluorophenyl)sulfonyl]1,3- | [M + H]⁺ |
| | (1,3-thiazol-4- | thiazol-4-ylcarbamate | |
| | yl)benzenesulfonamide | (WO2012004706) | |
| 230 | 5-chloro-4-(3,4- | tert-Butyl [(3-cyano-4- | m/z 401 |
| | difluorophenoxy)-2-fluoro-N- | fluorophenyl)sulfonyl]1,3-thiazol- | [M + H]⁺ |
| | (1,3-thiazol-4- | 4-ylcarbamate (WO2010079443) | |
| | yl)benzenesulfonamide | | |
| 231 | 4-(3-chloro-4-cyanophenoxy)- | tert-Butyl 1,3-thiazol-4-yl[(2,4,5- | m/z 426 |
| | 2,5-difluoro-N-(1,3-thiazol-4- | trifluorophenyl)sulfonyl]carbamate | [M − H]⁻ |
| | yl)benzenesulfonamide | (WO2012004706) | |
| 232 | 4-(3-chloro-4-cyanophenoxy)- | tert-butyl [(3,4- | m/z 408 |
| | 3-fluoro-N-(1,3-thiazol-2- | difluorophenyl)sulfonyl]1,3- | [M − H]⁻ |
| | yl)benzenesulfonamide | thiazol-2-ylcarbamate | |
| | | (WO2010079443) | |
| 233 | 4-(3-cyano-4-fluorophenoxy)-2- | tert-butyl [(2,4- | m/z 394 |
| | fluoro-N-(1,3-thiazol-4- | difluorophenyl)sulfonyl]1,3- | [M + H]⁺ |
| | yl)benzenesulfonamide | thiazol-4-ylcarbamate | |
| 234 | 5-bromo-4-(3,4- | tert-butyl [(5-bromo-2,4- | m/z 465 |
| | difluorophenoxy)-2-fluoro-N- | difluorophenyl)sulfonyl]1,3- | [M + H]⁺ |
| | (1,3-thiazol-4- | thiazol-4-ylcarbamate | |
| | yl)benzenesulfonamide | | |
| 235 | 5-chloro-4-(3-cyano-4- | tert-Butyl [(5-chloro-2,4- | m/z 428 |
| | fluorophenoxy)-2-fluoro-N-(1,3- | difluorophenyl)sulfonyl]1,3- | [M + H]⁺ |
| | thiazol-4- | thiazol-4-ylcarbamate | |
| | yl)benzenesulfonamide | (WO2012004706) | |
| 236 | 2-chloro-4-(3-cyano-4- | tert-butyl [(4-fluoro-2-chloro- | m/z 410 |
| | fluorophenoxy)-N-(1,3-thiazol- | phenyl)sulfonyl]1,3-thiazol-4- | [M + H]⁺ |
| | 4-yl)benzenesulfonamide | ylcarbamate | |
| 237 | 2-chloro-4-(3,4- | tert-butyl [(2-chloro-4- | m/z 403 |
| | difluorophenoxy)-N-(1,3- | fluorophenyl)sulfonyl]1,3-thiazol- | [M + H]⁺ |
| | thiazol-4- | 4-ylcarbamate | |
| | yl)benzenesulfonamide | | |
| 238 | 4-(3,4-difluorophenoxy)-2,5- | tert-Butyl 1,3-thiazol-4-yl[(2,4,5- | m/z 405 |
| | difluoro-N-(1,3-thiazol-4- | trifluorophenyl)sulfonyl]carbamate | [M + H]⁺ |
| | yl)benzenesulfonamide | (WO2012004706) | |
| 239 | 5-chloro-4-(3-chloro-4- | tert-Butyl [(5-chloro-2,4- | m/z 442 |
| | cyanophenoxy)-2-fluoro-N- | difluorophenyl)sulfonyl]1,3- | [M − H]⁻ |
| | (1,3-thiazol-4- | thiazol-4-ylcarbamate | |
| | yl)benzenesulfonamide | (WO2012004706) | |
| 240 | 4-(3-cyano-4-fluorophenoxy)-3- | tert-butyl [(4-fluoro-3- | m/z 502 |
| | iodo-N-(1,3-thiazol-4- | iodophenyl)sulfonyl]1,3-thiazol-4- | [M + H]⁺ |
| | yl)benzenesulfonamide | ylcarbamate | |
| 241 | 4-(3,4-difluorophenoxy)-3-iodo- | tert-butyl [(4-fluoro-3- | m/z 495 |
| | N-(thiazol-4- | iodophenyl)sulfonyl]1,3-thiazol-4- | [M + H]⁺ |
| | yl)benzenesulfonamide | ylcarbamate | |
| 242 | 4-(3-cyano-4-fluorophenoxy)-3- | tert-butyl [(3,4- | m/z 394 |
| | fluoro-N-(1,3-thiazol-2- | difluorophenyl)sulfonyl]1,3- | [M + H]⁺ |
| | yl)benzenesulfonamide | thiazol-2-ylcarbamate | |
| | | (WO2010079443) | |

### Library Protocol 3.1

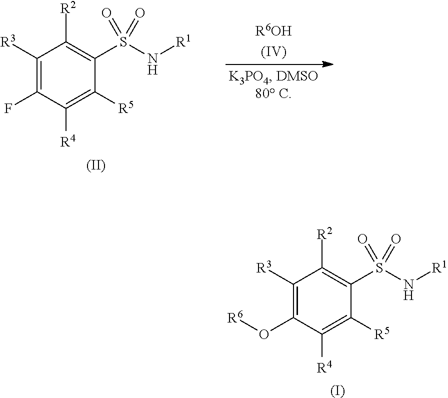

A 0.2M solution in DMSO of the compound of formula (IV) (500 μL, 100 μmol) was added to a 0.2M solution in DMSO of the compound of formula (II) (500 μL, 100 μmol) followed by anhydrous potassium phosphate (64 mg, 300 μmol). The reaction mixture was heated to 80° C. for 16 hours before concentrating in vacuo. The residue was dissolved in DMSO (1 mL) and purified using preparative HPLC as described below to afford the desired compound of formula (I).

### Preparative HPLC Method 1:

Mobile phase A: 10 mM ammonium acetate in water; Mobile phase B: MeCN

Column: Xterra RP18C18 (250×19 mm×10 u) or Reprosil Gold C18 (20×250 mm, 5 u)

Gradient: Initial 10% B; 3 mins 30% B; 18 mins 60% B, 19 mins 95% B, 22-25 mins 10% B

Flow rate: 20 mL/min.

### Preparative HPLC Method 2:

Mobile phase A: 20 mM ammonium bicarbonate in water; Mobile phase B: MeCN

Column: Gemini NX C18 (20×100 mm, 5 u)

Gradient: Initial 10% B; 2 mins 40% B; 10 mins 70% B, 11 mins 95% B, 13-15 mins 10% B

Flow rate: 20 mL/min

### LCMS Conditions:

Mobile phase A: 0.05% formic acid in water; Mobile phase B: MeCN

Column: RESTEK C18 2.1×30 mm×3μ

Gradient: From 98% [A] and 2% [B] to 90% [A] and 10% [B] in 1 min, further to 2% [A] and 98% [B] in 2.0 min and finally back to initial condition in 3 min

Flow rate: 1.5 mL/min

The compounds of the Examples in the table below were prepared from the appropriate sulphonamide and: (a) 3-cyano-4-fluorophenol; (b) 3,4-difluorophenol or (c) 3-chloro-4-cyanophenol; according to Library Protocol 3.1.

| Ex | Name | Sulfonamide | MS Data |
|---|---|---|---|
| 243 | 3-cyano-4-(3,4- | 3-cyano-4-fluoro-N-(1,3-thiazol- | m/z 394 |
| | difluorophenoxy)-N-(1,3-thiazol- | 2-yl)benzenesulfonamide | [M + H]⁺ |
| | 2-yl)benzenesulfonamide | (WO2012004743) | |
| 244 | 4-(3-chloro-4-cyanophenoxy)-3- | 3-cyano-4-fluoro-N-(5-fluoro-1,3- | m/z 435 |
| | cyano-N-(5-fluoro-1,3-thiazol-2- | thiazol-2-yl)benzenesulfonamide | [M + H]⁺ |
| | yl)benzenesulfonamide | (WO2012004743) | |
| 245 | 3-cyano-4-(3-cyano-4- | 3-cyano-4-fluoro-N-(5-fluoro-1,3- | m/z 419 |
| | fluorophenoxy)-N-(5-fluoro-1,3- | thiazol-2-yl)benzenesulfonamide | [M + H]⁺ |
| | thiazol-2-yl)benzenesulfonamide | (WO2012004743) | |
| 246 | 3-cyano-4-(3-cyano-4- | 3-cyano-4-fluoro-N-(1,3-thiazol- | m/z 399 |
| | fluorophenoxy)-N-(1,3-thiazol-2- | 2-yl)benzenesulfonamide | [M − H]⁻ |
| | yl)benzenesulfonamide | (WO2012004743) | |
| 247 | 4-(3-chloro-4-cyanophenoxy)-N- | 3-cyano-4-fluoro-N-(1,3-thiazol- | m/z 449 |
| | (5-chloro-1,3-thiazol-2-yl)-3- | 2-yl)benzenesulfonamide | [M − H]⁻ |
| | cyanobenzenesulfonamide | (WO2012004743) | |
| 248 | 3-cyano-4-(3,4- | N-(5-chloro-1,3-thiazol-2-yl)-3- | m/z 412 |
| | difluorophenoxy)-N-(5-fluoro- | cyano-4- | [M + H]⁺ |
| | 1,3-thiazol-2- | fluorobenzenesulfonamide | |
| | yl)benzenesulfonamide | (WO2010079443) | |

### Library Protocol 3.2

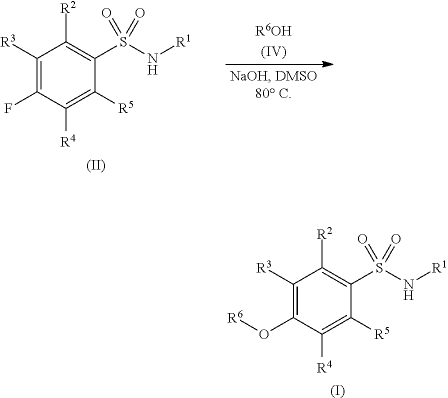

A 0.2M solution in DMSO of the compound of formula (IV) (500 μL, 100 μmol) was added to a 0.2M solution in DMSO of the compound of formula (II) (500 μL, 100 μmol) followed by sodium hydride (60% suspension in mineral oil, 9 mg, 200 μmol). The reaction mixture was heated to 80° C. for 16 hours before concentrating in vacuo. The residue was dissolved in DMSO (1 mL) and purified using preparative HPLC as described below to afford the desired compound of formula (I).

### Preparative HPLC Method 1:

Mobile phase A: 10 mM ammonium acetate in water; Mobile phase B: MeCN

Column: Xterra RP18C18 (250×19 mm×10 u) or Reprosil Gold C18 (20×250 mm, 5 u)

Gradient: Initial 10% B; 3 mins 30% B; 18 mins 60% B, 19 mins 95% B, 22-25 mins 10% B

Flow rate: 20 mL/min.

### Preparative HPLC Method 2:

Mobile phase A: 20 mM ammonium bicarbonate in water; Mobile phase B: MeCN

Column: Gemini NX C18 (20×100 mm, 5 u)

Gradient: Initial 10% B; 2 mins 40% B; 10 mins 70% B, 11 mins 95% B, 13-15 mins 10% B,

Flow rate: 20 mL/min

### LCMS Conditions:

Mobile phase A: 0.05% formic acid in water; Mobile phase B: MeCN

Column: RESTEK C18 2.1×30 mm×3μ

Gradient: From 98% [A] and 2% [B] to 90% [A] and 10% [B] in 1 min, further to 2% [A] and 98% [B] in 2.0 min and finally back to initial condition in 3 min

Flow rate: 1.5 mL/min

The compounds of the Examples in the table below were prepared from the appropriate sulphonamide and either 3,4-difluorophenol or 3-chloro-4-cyanophenol according to Library Protocol 3.2.

| Ex | Name | Sulphonamide | MS Data |
|---|---|---|---|
| 249 | 2-chloro-4-(3,4-difluorophenoxy)-N- | 2-chloro-4-fluoro-N-(1,3,4- | m/z 404 |
| | (1,3,4-thiadiazol-2- | thiadiazol-2- | [M + H]⁺ |
| | yl)benzenesulfonamide | yl)benzenesulfonamide | |
| 250 | 3-bromo-4-(3-chloro-4- | 3-bromo-4,5-difluoro-N- | m/z 489 |
| | cyanophenoxy)-5-fluoro-N-(1,2,4- | (1,2,4-thiadiazol-5- | [M + H]⁺ |
| | thiadiazol-5-yl)benzenesulfonamide | yl)benzenesulfonamide | |

### Library Protocol 4

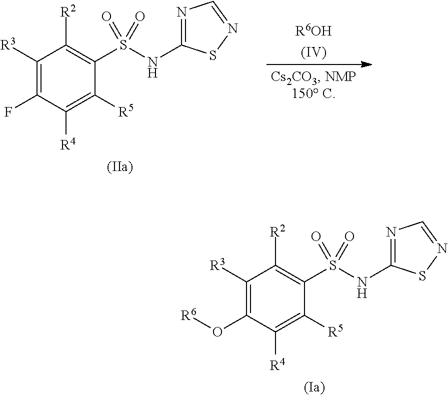

A 0.1875M solution in NMP of the compound of formula (I!a) (400 μL, 75 μmol) was added to the compound of formula (IV) (90 μmol) followed by cesium carbonate (47 mg, 150 μmol). The reaction mixture was shaken and heated to 150° C. for 3 hours before cooling and concentrating in vacuo. The residue was dissolved in DMSO (1 mL) and purified using preparative HPLC as described below to afford the desired compound of formula (Ia).

### Preparative HPLC/LCMS Conditions:

Mobile phase A:0.05% TFA in water; Mobile phase B: MeCN

Column: Welch XB-C18 (2.1×50 mm, 5 u)

The compounds of the Examples in the table below were prepared from 3-cyano-4-fluoro-N-(1,2,4-thiadiazol-5-yl)benzenesulfonamide (WO2012004743) and the appropriate phenol according to Library Protocol 4.

| Ex | Name | Alcohol | MS Data |
|---|---|---|---|
| 251 | 4-(2-chloro-6-fluorophenoxy)-3-cyano-N-(1,2,4- | 2-chloro-6- | m/z 411 |
| | thiadiazol-5-yl)benzenesulfonamide | fluorophenol | [M + H]⁺ |
| 252 | 3-cyano-4-[(5-methylpyridin-3-yl)oxy]-N-(1,2,4- | 5-methylpyridin- | m/z 374 |
| | thiadiazol-5-yl)benzenesulfonamide | 3-ol | [M + H]⁺ |
| | trifluoroacetic acid salt | | |
| 253 | 3-cyano-4-(pyridin-3-yloxy)-N-(1,2,4-thiadiazol- | 3- | m/z 360 |
| | 5-yl)benzenesulfonamide trifluoroacetic acid salt | hydroxypyridine | [M + H]⁺ |
| 254 | 3-cyano-4-{[2-(methylsulfanyl)pyridin-3-yl]oxy}- | [2- | m/z 406 |
| | N-(1,2,4-thiadiazol-5-yl)benzenesulfonamide | (methylsulfanyl)pyridin- | [M + H]⁺ |
| | trifluoroacetic acid salt | 3-ol | |
| 255 | 3-cyano-4-{[2-(ethylsulfanyl)pyridin-3-yl]oxy}-N- | [2- | m/z 420 |
| | (1,2,4-thiadiazol-5-yl)benzenesulfonamide | (ethylsulfanyl)pyridin- | [M + H]⁺ |
| | trifluoroacetic acid salt | 3-ol | |
| 256 | 3-cyano-4-(4-methoxy-2-methylphenoxy)-N- | 4-methoxy-2- | m/z 403 |
| | (1,2,4-thiadiazol-5-yl)benzenesulfonamide | methylphenol | [M + H]⁺ |
| 257 | 3-cyano-4-(3-fluoro-4-methoxyphenoxy)-N- | 3-fluoro-4- | m/z 407 |
| | (1,2,4-thiadiazol-5-yl)benzenesulfonamide | methoxyphenol | [M + H]⁺ |
| 258 | 3-cyano-4-(4-methylphenoxy)-N-(1,2,4- | 4-methylphenol | m/z 373 |
| | thiadiazol-5-yl)benzenesulfonamide | | [M + H]⁺ |
| 259 | 3-cyano-4-(2-ethoxy-4-methylphenoxy)-N- | 2-ethoxy-4- | m/z 417 |
| | (1,2,4-thiadiazol-5-yl)benzenesulfonamide | methylphenol | [M + H]⁺ |
| 260 | 3-cyano-4-(3-methylphenoxy)-N-(1,2,4- | 3-methylphenol | m/z 373 |
| | thiadiazol-5-yl)benzenesulfonamide | | [M + H]⁺ |
| 261 | 4-(2-chloro-5-methoxyphenoxy)-3-cyano-N- | 2-chloro-5- | m/z 423 |
| | (1,2,4-thiadiazol-5-yl)benzenesulfonamide | methoxyphenol | [M + H]⁺ |
| 262 | 3-cyano-4-[5-fluoro-2-(propan-2-yloxy)phenoxy]- | 5-fluoro-2- | m/z 435 |
| | N-(1,2,4-thiadiazol-5-yl)benzenesulfonamide | (propan-2- | [M + H]⁺ |
| | | yloxy)phenol | |
| 263 | 4-(2-chloro-6-methoxyphenoxy)-3-cyano-N- | 2-chloro-6- | m/z 423 |
| | (1,2,4-thiadiazol-5-yl)benzenesulfonamide | methoxyphenol | [M + I.H]⁺ |
| 264 | 3-cyano-4-(4-fluoro-3-methoxy-2- | 4-fluoro-3- | m/z 421 |
| | methylphenoxy)-N-(1,2,4-thiadiazol-5- | methoxy-2- | [M + H]⁺ |
| | yl)benzenesulfonamide | methylphenol | |
| 265 | 3-cyano-4-(2-fluoro-6-methoxyphenoxy)-N- | 2-fluoro-6- | m/z 407 |
| | (1,2,4-thiadiazol-5-yl)benzenesulfonamide | methoxyphenol | [M + H]⁺ |
| 266 | 3-cyano-4-(4-fluoro-2-methoxy-3- | 4-fluoro-2- | m/z 421 |
| | methylphenoxy)-N-(1,2,4-thiadiazol-5- | methoxy-3- | [M + H]⁺ |
| | yl)benzenesulfonamide | methylphenol | |
| 267 | 4-(4-chloro-2-methoxyphenoxy)-3-cyano-N- | 4-chloro-2- | m/z 423 |
| | (1,2,4-thiadiazol-5-yl)benzenesulfonamide | methoxyphenol | [M + H]⁺ |
| 268 | 4-(3-chloro-4-methoxyphenoxy)-3-cyano-N- | 3-chloro-4- | m/z 423 |
| | (1,2,4-thiadiazol-5-yl)benzenesulfonamide | methoxyphenol | [M + H]⁺ |
| 269 | 4-(3-chloro-5-methoxyphenoxy)-3-cyano-N- | 3-chloro-5- | m/z 423 |
| | (1,2,4-thiadiazol-5-yl)benzenesulfonamide | methoxyphenol | [M + H]⁺ |
| 270 | 3-cyano-4-(4,5-difluoro-2-methoxyphenoxy)-N- | 5-difluoro-2- | m/z 425 |
| | (1,2,4-thiadiazol-5-yl)benzenesulfonamide | methoxyphenol | [M + H]⁺ |
| 271 | 3-cyano-4-(2-methoxyphenoxy)-N-(1,2,4- | 2- | m/z 389 |
| | thiadiazol-5-yl)benzenesulfonamide | methoxyphenol | [M + H]⁺ |
| 272 | 3-cyano-4-[(4-methylpyridin-3-yl)oxy]-N-(1,2,4- | 4-methyl-3- | m/z 374 |
| | thiadiazol-5-yl)benzenesulfonamide | hydroxypyridine | [M + H]⁺ |
| | trifluoroacetic acid salt | | |
| 273 | 3-cyano-4-[(2-ethylpyridin-3-yl)oxy]-N-(1,2,4- | 2-ethyl-3- | m/z 388 |
| | thiadiazol-5-yl)benzenesulfonamide | hydroxypyridine | [M + H]⁺ |
| | trifluoroacetic acid salt | | |
| 274 | 3-cyano-4-{[2-(propan-2-yl)pyridin-3-yl]oxy}-N- | 2-isopropyl-3- | m/z 402 |
| | (1,2,4-thiadiazol-5-yl)benzenesulfonamide | hydroxypyridine | [M + H]⁺ |
| | trifluoroacetic acid salt | | |
| 275 | 3-cyano-4-[(2,4-dimethylpyridin-3-yl)oxy]-N- | 2,4-dimethyl-3- | m/z 388 |
| | (1,2,4-thiadiazol-5-yl)benzenesulfonamide | hydroxypyridine | [M + H]⁺ |
| | trifluoroacetic acid salt | | |
| 276 | 3-cyano-4-[(2-ethyl-6-methylpyridin-3-yl)oxy]-N- | 2-ethyl-6- | m/z 402 |
| | (1,2,4-thiadiazol-5-yl)benzenesulfonamide | methyl-3- | [M + H]⁺ |
| | trifluoroacetic acid salt | hydroxypyridine | |
| 277 | 3-cyano-4-(5-fluoro-2-methoxyphenoxy)-N- | 5-fluoro-2- | m/z 407 |
| | (1,2,4-thiadiazol-5-yl)benzenesulfonamide | methoxyphenol | [M + H]⁺ |
| 278 | 3-cyano-4-[2-(propan-2-yloxy)phenoxy]-N- | 2-isopropoxy | m/z 417 |
| | (1,2,4-thiadiazol-5-yl)benzenesulfonamide | phenol | [M + H]⁺ |
| 279 | 3-cyano-4-(4-ethoxyphenoxy)-N-(1,2,4- | 4-ethoxyphenol | m/z 403 |
| | thiadiazol-5-yl)benzenesulfonamide | | [M + H]⁺ |
| 280 | 3-cyano-4-(3-ethoxyphenoxy)-N-(1,2,4- | 3-ethoxyphenol | m/z 403 |
| | thiadiazol-5-yl)benzenesulfonamide | | [M + H]⁺ |
| 281 | 3-cyano-4-(2-ethoxyphenoxy)-N-(1,2,4- | 2-ethoxyphenol | m/z 403 |
| | thiadiazol-5-yl)benzenesulfonamide | | [M + H]⁺ |
| 282 | 3-cyano-4-(2,6-difluoro-3-methoxyphenoxy)-N- | 2,6-difluoro-3- | m/z 425 |
| | (1,2,4-thiadiazol-5-yl)benzenesulfonamide | methoxyphenol | [M + H]⁺ |
| 283 | 4-(3-chloro-2-methoxyphenoxy)-3-cyano-N- | 3-chloro-2- | m/z 423 |
| | (1,2,4-thiadiazol-5-yl)benzenesulfonamide | methoxyphenol | [M + H]⁺ |
| 284 | 3-cyano-4-(2-methoxy-5-methylphenoxy)-N- | 2-methoxy-5- | m/z 403 |
| | (1,2,4-thiadiazol-5-yl)benzenesulfonamide | methylphenol | [M + H]⁺ |
| 285 | 3-cyano-4-(2-methoxy-4-methylphenoxy)-N- | 2-methoxy-4- | m/z 403 |
| | (1,2,4-thiadiazol-5-yl)benzenesulfonamide | methylphenol | [M + H]⁺ |
| 286 | 3-cyano-4-(4-fluoro-2-methoxyphenoxy)-N- | 4-fluoro-2- | m/z 407 |
| | (1,2,4-thiadiazol-5-yl)benzenesulfonamide | methoxyphenol | [M + H]⁺ |
| 287 | 3-cyano-4-(2-fluoro-5-methoxyphenoxy)-N- | 2-fluoro-5- | m/z 407 |
| | (1,2,4-thiadiazol-5-yl)benzenesulfonamide | methoxyphenol | [M + H]⁺ |
| 288 | 3-cyano-4-(3,4-difluoro-2-methylphenoxy)-N- | 3,4-difluoro-2- | m/z 409 |
| | (1,2,4-thiadiazol-5-yl)benzenesulfonamide | methylphenol | [M + H]⁺ |
| 289 | 3-cyano-4-(3-fluoro-5-methoxyphenoxy)-N- | 3-fluoro-5- | m/z 407 |
| | (1,2,4-thiadiazol-5-yl)benzenesulfonamide | methoxyphenol | [M + H]⁺ |
| 290 | 4-(2-chloro-4-methoxyphenoxy)-3-cyano-N- | 2-chloro-4- | m/z 423 |
| | (1,2,4-thiadiazol-5-yl)benzenesulfonamide | methoxyphenol | [M + H]⁺ |
| 291 | 4-(3-chlorophenoxy)-3-cyano-N-(1,2,4- | 3-chlorophenol | m/z 393 |
| | thiadiazol-5-yl)benzenesulfonamide | | [M + H]⁺ |
| 292 | 3-cyano-4-(3-methoxy-5-methylphenoxy)-N- | 3-methoxy-5- | m/z 403 |
| | (1,2,4-thiadiazol-5-yl)benzenesulfonamide | methylphenol | [M + H]⁺ |
| 293 | 3-cyano-4-(3-methoxy-2-methylphenoxy)-N- | 3-methoxy-2- | m/z 403 |
| | (1,2,4-thiadiazol-5-yl)benzenesulfonamide | methylphenol | [M + H]⁺ |
| 294 | 3-cyano-4-(4-fluorophenoxy)-N-(1,2,4- | 4-fluorophenol | m/z 377 |
| | thiadiazol-5-yl)benzenesulfonamide | | [M + H]⁺ |

### Library Protocol 5

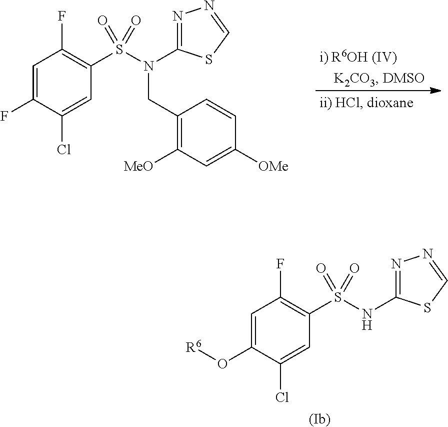

### Step (i)

A 0.15M solution of 5-chloro-N-(2,4-dimethoxybenzyl)-2,4-difluoro-N-1,3,4-thiadiazol-2-ylbenzenesulfonamide (WO2012004743, 5 μL, 75 μmol) in DMSO was added to the compound of formula (IV) (75 μmol) followed by potassium carbonate (21 mg, 150 μmol). The reaction mixture was shaken at 30° C. for 16 hours. The reaction mixture was filtered before concentrating in vacuo to afford an intermediate residue.

### Step (ii)

To the intermediate residue was added a 4M HCl solution in dioxane (1 mL) and the reaction mixture was shaken at 30° C. for 1 hour before concentrating in vacuo to afford a further residue. This further residue was dissolved in DMSO and purified by preparative HPLC to afford the desired compound of formula (I).

### LCMS Conditions:

Column: Welch XB-C18 2.1×50 mm 5 μm

Mobile phase A: 0.0375% TFA in water; mobile phase B: 0.01875% TFA in acetonitrile.

Initial gradient either 1, 10 or 25% B; 3.50-4.00 mins 100% B, 4.70 mins return to 1, 10 or 25% B. Flow rate 0.8 mL/min.

### Preparative HPLC Conditions:

### Method A:

Phenomenex Gemini C18 eluting with acetonitrile-ammonium hydroxide with an organic gradient of between 15-56% and a flow rate of 25-30 mL/min.

Method B:

Grace Vydac C18 200×20 mm×5 um eluting with acetonitrile-water (0.1% TFA) with an organic gradient of between 28-68% and a flow rate of 25 mL/min.

The compounds of the Examples in the table below were prepared from 5-chloro-N-(2,4-dimethoxybenzyl)-2,4-difluoro-N-1,3,4-thiadiazol-2-ylbenzenesulfonamide (WO2012004743) and the appropriate alcohol according to Library Protocol 5 and purified by Purification Method A.

| Ex | Name | Alcohol | MS Data |
|---|---|---|---|
| 295 | 5-chloro-2-fluoro-4-[5-fluoro-2-(propan-2- | 5-fluoro-2- | m/z 462 |
| | yloxy)phenoxy]-N-(1,3,4-thiadiazol-2- | (propan-2- | [M + H]⁺ |
| | yl)benzenesulfonamide | yloxy)phenol | |
| 296 | 5-chloro-4-(4-cyano-2-methoxyphenoxy)-2- | 4-cyano-2- | m/z 441 |
| | fluoro-N-(1,3,4-thiadiazol-2- | methoxyphenol | [M + H]⁺ |
| | yl)benzenesulfonamide | | |
| 297 | 5-chloro-2-fluoro-4-(3-fluoro-5- | 3-fluoro-5- | m/z 434 |
| | methoxyphenoxy)-N-(1,3,4-thiadiazol-2- | methoxyphenol | [M + H]⁺ |
| | yl)benzenesulfonamide | | |
| 298 | 5-chloro-2-fluoro-4-{[2-(propan-2-yl)pyridin-3- | 2-(propan-2-yl)- | m/z 429 |
| | yl]oxy}-N-(1,3,4-thiadiazol-2- | 3- | [M + H]⁺ |
| | yl)benzenesulfonamide | hydroxypyridine | |
| 299 | 5-chloro-2-fluoro-4-(2-methoxy-4- | 2-methoxy-4- | m/z 430 |
| | methylphenoxy)-N-(1,3,4-thiadiazol-2- | methylphenol | [M + H]⁺ |
| | yl)benzenesulfonamide | | |
| 300 | 5-chloro-2-fluoro-4-(2-methoxy-5- | 2-methoxy-5- | m/z 430 |
| | methylphenoxy)-N-(1,3,4-thiadiazol-2- | methylphenol | [M + H]⁺ |
| | yl)benzenesulfonamide | | |
| 301 | 5-chloro-2-fluoro-4-(3-methoxy-5- | 3-methoxy-5- | m/z 430 |
| | methylphenoxy)-N-(1,3,4-thiadiazol-2- | methylphenol | [M + H]⁺ |
| | yl)benzenesulfonamide | | |
| 302 | 5-chloro-4-(3-chloro-5-methoxyphenoxy)-2- | 3-chloro-5- | m/z 450 |
| | fluoro-N-(1,3,4-thiadiazol-2- | methoxyphenol | [M]⁺ |
| | yl)benzenesulfonamide | | |
| 303 | 5-chloro-4-(2-chloro-6-methoxyphenoxy)-2- | 2-chloro-6- | m/z 450 |
| | fluoro-N-(1,3,4-thiadiazol-2- | methoxyphenol | [M]⁺ |
| | yl)benzenesulfonamide | | |
| 304 | 5-chloro-4-{[2-(ethylsulfanyl)pyridin-3-yl]oxy}-2- | 2-(ethylsulfanyl)pyridin- | m/z 447 |
| | fluoro-N-(1,3,4-thiadiazol-2- | 3-ol | [M + H]⁺ |
| | yl)benzenesulfonamide | | |
| 305 | 5-chloro-2-fluoro-4-(2-methoxyphenoxy)-N- | 2- | m/z 416 |
| | (1,3,4-thiadiazol-2-yl)benzenesulfonamide | methoxyphenol | [M + H]⁺ |
| 306 | 5-chloro-2-fluoro-4-(3-methoxyphenoxy)-N- | 3- | m/z 416 |
| | (1,3,4-thiadiazol-2-yl)benzenesulfonamide | methoxyphenol | [M + H]⁺ |
| 307 | 5-chloro-4-[(2-ethylpyridin-3-yl)oxy]-2-fluoro-N- | 2-ethyl-3- | m/z 415 |
| | (1,3,4-thiadiazol-2-yl)benzenesulfonamide | hydroxypyridine | [M + H]⁺ |
| 308 | 5-chloro-4-[(2,4-dimethylpyridin-3-yl)oxy]-2- | 2,4-dimethyl-3- | m/z 415 |
| | fluoro-N-(1,3,4-thiadiazol-2- | hydroxypyridine | [M + H]⁺ |
| | yl)benzenesulfonamide | | |
| 309 | 5-chloro-4-(5-cyano-2-methoxyphenoxy)-2- | 5-cyano-2- | m/z 441 |
| | fluoro-N-(1,3,4-thiadiazol-2- | methoxyphenol | [M + H]⁺ |
| | yl)benzenesulfonamide | | |
| 310 | 5-chloro-2-fluoro-4-{[2-(methylsulfanyl)pyridin-3- | 2- | m/z 433 |
| | yl]oxy}-N-(1,3,4-thiadiazol-2- | (methylsulfanyl)pyridin- | [M + H]⁺ |
| | yl)benzenesulfonamide | 3-ol | |
| 311 | 5-chloro-2-fluoro-4-[(5-methylpyridin-3-yl)oxy]-N- | 5-methyl-3- | m/z 401 |
| | (1,3,4-thiadiazol-2-yl)benzenesulfonamide | hydroxypyridine | [M + H]⁺ |
| 312 | 5-chloro-2-fluoro-4-(4-fluoro-2- | 4-fluoro-2- | m/z 434 |
| | methoxyphenoxy)-N-(1,3,4-thiadiazol-2- | methoxyphenol | [M + H]⁺ |
| | yl)benzenesulfonamide | | |
| 313 | 5-chloro-2-fluoro-4-[(4-methylpyridin-3-yl)oxy]-N- | 4-methyl-3- | m/z 401 |
| | (1,3,4-thiadiazol-2-yl)benzenesulfonamide | hydroxypyridine | [M + H]⁺ |
| 314 | 5-chloro-2-fluoro-4-(4-methylphenoxy)-N-(1,3,4- | 4-methylphenol | m/z 400 |
| | thiadiazol-2-yl)benzenesulfonamide | | [M + H]⁺ |
| 315 | 5-chloro-2-fluoro-4-(2-fluoro-5- | 2-fluoro-5- | m/z 434 |
| | methoxyphenoxy)-N-(1,3,4-thiadiazol-2- | methoxyphenol | [M + H]⁺ |
| | yl)benzenesulfonamide | | |
| 316 | 5-chloro-4-(4-cyano-5-fluoro-2- | 4-cyano-5- | m/z 459 |
| | methoxyphenoxy)-2-fluoro-N-(1,3,4-thiadiazol-2- | fluoro-2- | [M + H]⁺ |
| | yl)benzenesulfonamide | methoxyphenol | |
| 317 | 5-chloro-4-(2-chloro-4-methoxyphenoxy)-2- | 2-chloro-4- | m/z 449 |
| | fluoro-N-(1,3,4-thiadiazol-2- | methoxyphenol | [M]⁺ |
| | yl)benzenesulfonamide | | |
| 318 | 5-chloro-4-(3,4-difluoro-2-methylphenoxy)-2- | 3,4-difluoro-2- | m/z 436 |
| | fluoro-N-(1,3,4-thiadiazol-2- | methylphenol | [M + H]⁺ |
| | yl)benzenesulfonamide | | |
| 319 | 5-chloro-2-fluoro-4-(4-fluoro-2-methoxy-3- | 4-fluoro-2- | m/z 448 |
| | methylphenoxy)-N-(1,3,4-thiadiazol-2- | methoxy-3- | [M + H]⁺ |
| | yl)benzenesulfonamide | methylphenol | |
| 320 | 5-chloro-4-(3-chloro-4-methoxyphenoxy)-2- | 3-chloro-4- | m/z 449 |
| | fluoro-N-(1,3,4-thiadiazol-2- | methoxyphenol | [M]⁺ |
| | yl)benzenesulfonamide | | |
| 321 | 5-chloro-2-fluoro-4-(2-methoxy-6- | 2-methoxy-6- | m/z 430 |
| | methylphenoxy)-N-(1,3,4-thiadiazol-2- | methylphenol | [M + H]⁺ |
| | yl)benzenesulfonamide | | |
| 322 | 5-chloro-2-fluoro-4-(4-fluoro-3-methylphenoxy)- | 4-fluoro-3- | m/z 418 |
| | N-(1,3,4-thiadiazol-2-yl)benzenesulfonamide | methylphenol | [M + H]⁺ |
| 323 | 5-chloro-2-fluoro-4-(3-methoxy-2- | 3-methoxy-2- | m/z 430 |
| | methylphenoxy)-N-(1,3,4-thiadiazol-2- | methylphenol | [M + H]⁺ |
| | yl)benzenesulfonamide | | |
| 324 | 5-chloro-2-fluoro-4-(3-fluoro-4- | 3-fluoro-4- | m/z 434 |
| | methoxyphenoxy)-N-(1,3,4-thiadiazol-2- | methoxyphenol | [M + H]⁺ |
| | yl)benzenesulfonamide | | |
| 325 | 5-chloro-2-fluoro-4-[2-(propan-2-yloxy)phenoxy]- | 2-(propan-2- | m/z 444 |
| | N-(1,3,4-thiadiazol-2-yl)benzenesulfonamide | yloxy)phenol | [M + H]⁺ |
| 326 | 5-chloro-4-(2-chloro-5-methoxyphenoxy)-2- | 2-chloro-5- | m/z 449 |
| | fluoro-N-(1,3,4-thiadiazol-2- | methoxyphenol | [M]⁺ |
| | yl)benzenesulfonamide | | |
| 327 | 5-chloro-2-fluoro-4-(5-methoxy-2- | 5-methoxy-2- | m/z 430 |
| | methylphenoxy)-N-(1,3,4-thiadiazol-2- | methylphenol | [M + H]⁺ |
| | yl)benzenesulfonamide | | |
| 328 | 5-chloro-2-fluoro-4-(2-methylphenoxy)-N-(1,3,4- | 2-methylphenol | m/z 400 |
| | thiadiazol-2-yl)benzenesulfonamide | | [M + H]⁺ |
| 329 | 5-chloro-4-(2-chloro-4-fluoro-3- | 2-chloro-4- | m/z 467 |
| | methoxyphenoxy)-2-fluoro-N-(1,3,4-thiadiazol-2- | fluoro-3- | [M]⁺ |
| | yl)benzenesulfonamide | methoxyphenol | |
| 330 | 5-chloro-4-(3-chlorophenoxy)-2-fluoro-N-(1,3,4- | 3-chlorophenol | m/z 419 |
| | thiadiazol-2-yl)benzenesulfonamide | | [M]⁺ |
| 331 | 5-chloro-4-(2-ethoxy-4-methylphenoxy)-2-fluoro- | 2-ethoxy-4- | m/z 444 |
| | N-(1,3,4-thiadiazol-2-yl)benzenesulfonamide | methylphenol | [M + H]⁺ |
| 332 | 5-chloro-4-(2-chloro-6-fluorophenoxy)-2-fluoro- | 2-chloro-6- | m/z 437 |
| | N-(1,3,4-thiadiazol-2-yl)benzenesulfonamide | fluorophenol | [M]⁺ |

### Library Protocol 6

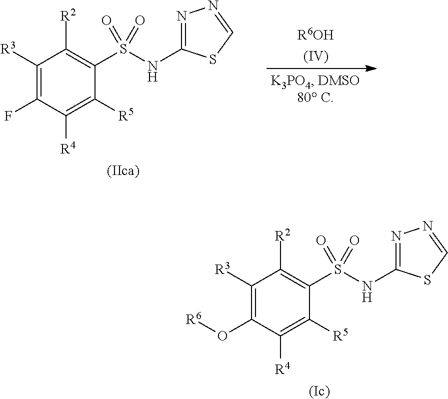

A 0.2M solution in DMSO of the compound of formula (IIc) (500 μL, 100 μmol) was added to a 0.2M solution in DMSO of the compound of formula (IV) (500 μL, 100 μmol) followed by the addition of potassium phosphate (64 mg, 3 eq). The reaction was heated to 80° C. for 16 hours. The reaction was cooled and purified directly using preparative HPLC as described below to afford the desired compound of formula (Ic).

### Preparative HPLC Method:

Mobile phase A: 20 mM ammonium bicarbonate in water; Mobile phase B: MeCN

Column: Gemini NX C18 (20×100 mm, 5 u) or YMC Triart C18 (30×100 mm, 5 u).

Gradient: Initial 10% B; 2 mins 30% B; 10 mins 70% B, 11-12 mins 95% B, 13-15 mins 10% B.

Flow rate: 30 mL/min.

### LCMS Conditions:

Mobile phase A: 0.05% formic acid in water; Mobile phase B: MeCN

Column: RESTEK C18 2.1×30 mm×3μ

Gradient: From 98% [A] and 2% [B] to 90% [A] and 10% [B] in 1 min, further to 2% [A] and 98% [B] in 2.0 min and finally back to initial condition in 3 min.

Flow rate: 1.5 mL/min.

The compounds of the Examples in the table below were prepared from 3-cyano-4-fluoro-N-1,3,4-thiadiazol-2-yl-benzenesulfonamide (Preparation 51) and the appropriate alcohol according to Library Protocol 6.

| Ex | Name/Structure | Alcohol | MS Data |
|---|---|---|---|
| 333 | 4-{[5-chloro-6-(propan-2-yloxy)pyridin- | 5-chloro-6-(1-propan-2- | m/z 450 |
| | 3-yl]oxy}-3-cyano-N-(1,3,4-thiadiazol-2- | yloxy)-3-pyridinol | [M − H]⁻ |
| | yl)benzenesulfonamide | (WO2012007877) | |
| 334 | 4-{[5-chloro-6-(difluoromethoxy)pyridin- | 5-chloro-6- | m/z 458 |
| | 3-yl]oxy}-3-cyano-N-(1,3,4-thiadiazol-2- | (difluoromethoxy)-3- | [M − H]⁻ |
| | yl)benzenesulfonamide | pyridinol | |
| | | (WO2012007869) | |
| 335 | Racemic 4-({5-chloro-6-[(1,1,1- | Racemic 5-chloro-6- | m/z 506 |
| | trifluoropropan-2-yl)oxy]pyridin-3- | [(1,1,1-trifluoropropan-2- | [M + H]⁺ |
| | yl}oxy)-3-cyano-N-(1,3,4-thiadiazol-2- | yl)oxy]-3-pyridinol | |
| | yl)benzenesulfonamide | (WO2012007869) | |
| 336 | 4-{[5-chloro-6-(cyclobutyloxy)pyridin-3- | 5-chloro-6-cyclobutoxy-3- | m/z 462 |
| | yl]oxy}-3-cyano-N-(1,3,4-thiadiazol-2- | pyridinol | [M − H]⁻ |
| | yl)benzenesulfonamide | (WO2012007869) | |
| 337 | 3-cyano-4-(4-cyano-3,5- | 4-hydroxy-2,6-dimethyl- | m/z 410 |
| | dimethylphenoxy)-N-(1,3,4-thiadiazol-2- | benzonitrile | [M − H]⁻ |
| | yl)benzenesulfonamide | | |
| 338 | 4-[(5-chloro-6-methoxypyridin-3-yl)oxy]- | 5-chloro-6-methoxy-3- | m/z 424 |
| | 3-cyano-N-(1,3,4-thiadiazol-2- | pyridinol | [M + H]⁺ |
| | yl)benzenesulfonamide | (WO2012007869) | |
| 339 | 4-{[5-chloro-6-(2-fluoro-2- | 5-chloro-6-(2-fluoro-2- | m/z 482 |
| | methylpropoxy)pyridin-3-yl]oxy}-3- | methylpropoxy)-3- | [M − H]⁻ |
| | cyano-N-(1,3,4-thiadiazol-2- | pyridinol | |
| | yl)benzenesulfonamide | (WO2012007869) | |

### Example 340

### 4-(3-chloro-4-cyanophenoxy)-3-cyano-N-(5-methyl-1,3,4-thiadiazol-2-yl)benzenesulfonamide

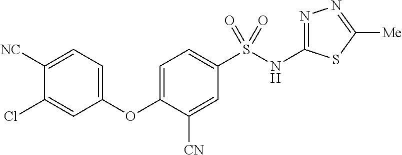 [SMILES: Cc1nnc(NS(=O)(=O)c2ccc(Oc3ccc(C#N)c(Cl)c3)c(C#N)c2)s1]

4-(3-chloro-4-cyanophenoxy)-3-cyanobenzene-1-sulfonyl chloride (Preparation 3, 40 mg, 0.11 mmol) and 5-methyl-1,3,4-thiadiazol-2-amine (14 mg, 0.12 mmol) were dissolved in DCM (2 mL). Pyridine (29 μL, 0.34 mmol) was added and the reaction was stirred at room temperature for 20 hours. The reaction was concentrated in vacuo and purified by preparative HPLC to afford the title compound.

MS m/z 432 [M+H]⁺

### Example 341

### 4-[3-chloro-4-(hydroxymethyl)phenoxy]-3-cyano-N-(1,3,4-thiadiazol-2-yl)benzenesulfonamide

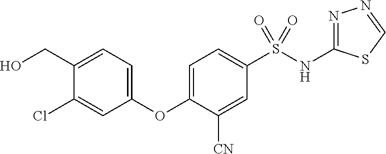 [SMILES: N#Cc1cc(S(=O)(=O)Nc2nncs2)ccc1Oc1ccc(CO)c(Cl)c1]

To a suspension of 4-(3-chloro-4-(hydroxymethyl)phenoxy)-3-cyano-N-(1,3,4-thiadiazol-2-yl)benzenesulfonamide (Preparation 52, 90 mg, 0.214 mmol) in methanol (2 mL) was added sodium borohydride (25 mg) and the reaction was stirred at room temperature for 30 minutes. The reaction was diluted with water (3 mL) and acidified with 1M HCl (2 mL). The solid was collected, dissolved in acetone (15 mL) and precipitated with water (60 mL). The solid was collected by filtration and dried under vacuum to afford the title compound (58 mg, 65%).

¹HNMR (400 MHz, DMSO-d₆): δ ppm 4.56 (s, 2H), 5.48 (br s, 1H), 7.01 (d, 1H), 7.27 (dd, 1H), 7.45 (d, 1H), 7.63 (d, 1H), 8.00 (dd, 1H), 8.25 (d, 1H), 8.79 (s, 1H).

MS m/z 423 [M+H]⁺

The compounds of formula (I) that follow, or pharmaceutically acceptable salts thereof, may be prepared by procedures described in the aforementioned: Schemes; General Methods and Method Variations, as further illustrated by the Examples and corresponding Preparations; or by processes similar thereto.
4-(3-Chloro-4-cyanophenoxy)-N-(1-methyl-1H-pyrazol-5-yl)benzenesulfonamide.
4-(3-Chloro-4-cyanophenoxy)-3-cyano-5-fluoro-N-(1,2,4-thiadiazol-5-yl)benzenesulfonamide.
{[4-(3-Chloro-4-cyanophenoxy)-3-fluorophenyl]sulfonyl}(1,2,4-thiadiazol-5-yl)azanide.
4-(3-Chloro-4-cyanophenoxy)-2-cyano-N-(1,2,4-thiadiazol-5-yl)benzenesulfonamide.
4-(3-Chloro-4-cyanophenoxy)-3-cyano-N-(1,3-oxazol-2-yl)benzenesulfonamide.
4-(3-Chloro-4-cyanophenoxy)-3-cyano-N-(1,3,4-oxadiazol-2-yl)benzenesulfonamide.
4-(3-Chloro-4-cyanophenoxy)-3-cyano-N-(1,2,4-oxadiazol-5-yl)benzenesulfonamide.
4-(3-Chloro-4-cyanophenoxy)-3-cyano-N-(1,2-oxazol-5-yl)benzenesulfonamide.
4-(3-Chloro-4-cyanophenoxy)-3-cyano-N-(1,2-oxazol-3-yl)benzenesulfonamide.
4-(3-Chloro-4-cyanophenoxy)-3-cyano-N-(5-methyl-1,3-thiazol-2-yl)benzenesulfonamide.
4-(3-Chloro-4-cyanophenoxy)-3-cyano-N-(3-methyl-1,2-thiazol-4-yl)benzenesulfonamide.
4-(3-Chloro-4-cyanophenoxy)-3-cyano-N-(5-fluoro-1,3,4-thiadiazol-2-yl)benzenesulfonamide.
4-(3-Chloro-4-cyanophenoxy)-N-(5-chloro-1,3,4-thiadiazol-2-yl)-3-cyanobenzenesulfonamide.
N-(5-tert-Butyl-1,3,4-thiadiazol-2-yl)-4-(3-chloro-4-cyanophenoxy)-3-cyanobenzenesulfonamide.
4-(3-Chloro-4-cyanophenoxy)-3-cyano-N-[5-(propan-2-yl)-1,3,4-thiadiazol-2-yl]benzenesulfonamide.
4-(3-Chloro-4-cyanophenoxy)-3-cyano-N-[5-(trifluoromethyl)-1,3,4-thiadiazol-2-yl]benzenesulfonamide.
4-(3-Chloro-4-cyanophenoxy)-3-cyano-N-(5-ethyl-1,3,4-thiadiazol-2-yl)benzenesulfonamide.

### Preparation 1

### 3-cyano-4-(2-fluoro-4-formylphenoxy)-N-(1,3-thiazol-2-yl)benzenesulfonamide

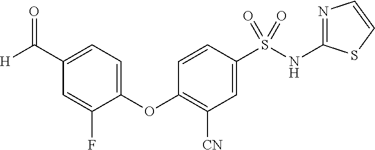 [SMILES: N#Cc1cc(S(=O)(=O)Nc2nccs2)ccc1Oc1ccc(C=O)cc1F]

The title compound was prepared in a manner analogous to that for the preparation of a compound of formula (I) by Method Variation 4 and Purification Method A (each as described hereinabove), using 2-fluoro-4-formylphenol and 3-cyano-4-fluoro-N-(1,3-thiazol-2-yl)benzenesulfonamide (WO2012004743).

MS m/z 402 [M−H]⁻

### Preparation 2

### 3-cyano-4-(4-bromo-2-formylphenoxy)-N-(1,3-thiazol-2-yl)benzenesulfonamide

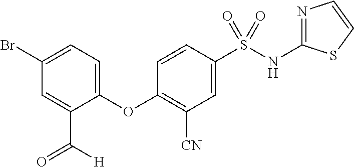 [SMILES: N#Cc1cc(S(=O)(=O)Nc2nccs2)ccc1Oc1ccc(Br)cc1C=O]

The title compound was prepared in a manner analogous to that for the preparation of a compound of formula (I) by Method Variation 1 (as described hereinabove) using 5-bromosalicylaldehyde and N-(2,4-dimethoxybenzyl)-3,4-difluoro-N-(1,3-thiazol-2-yl)benzenesulfonamide (WO2012004743).

### Preparation 3

### 4-(3-chloro-4-cyanophenoxy)-3-cyanobenzene-1-sulfonyl chloride

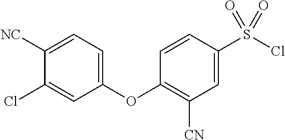 [SMILES: N#Cc1ccc(Oc2ccc(S(=O)(=O)Cl)cc2C#N)cc1Cl]

To a solution of trichloroisocyanuric acid (2.68 g, 11.55 mmol) in acetonitrile (25 mL) was added a solution of benzyltrimethylammonium chloride (6.56 g, 35.28 mmol) in water (11.7 mL). The mixture was stirred at room temperature for 30 minutes, then cooled with an ice bath. The mixture was added to an ice cold solution of 4-(4-(benzylthio)-2-cyanophenoxy)-2-chlorobenzonitrile (Preparation 4, 4.61 g, 10.50 mmol) in acetonitrile (50 mL) and 1M sodium carbonate (10.5 mL, 10.5 mmol) and stirred at this temperature for 30 minutes. The reaction was diluted with ethyl acetate and washed twice with dilute sodium hydrogen carbonate solution (2×200 mL). The combined organic layers were dried over magnesium sulfate, filtered and the solvent removed to leave a yellow solid (4.9 g). The crude residue was purified using silica gel column chromatography eluting with 0% to 20% ethyl acetate in heptanes to afford the title compound (3.05 g, 46%).

¹HNMR (400 MHz, CDCl₃): δ ppm 7.13 (d, 1H), 7.19 (d, 1H), 7.36 (s, 1H), 7.83 (d, 1H), 8.22 (d, 1H), 8.41 (s, 1H)

### Preparation 4

### 4-(4-(benzylthio)-2-cyanophenoxy)-2-chlorobenzonitrile

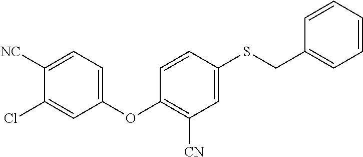 [SMILES: N#Cc1ccc(Oc2ccc(SCc3ccccc3)cc2C#N)cc1Cl]

To a solution of 5-(benzylthio)-2-fluorobenzonitrile (Preparation 5, 6.18 g, 25.4 mmol) in dimethylsulfoxide (64 mL) was added potassium carbonate (10.92 g, 79 mmol) and 2-fluoro-4-hydroxybenzonitrile (6.07 g, 39.5 mmol). The reaction mixture was heated at 80° C. for 18 hours. The reaction mixture was diluted to 500 mL volume with diethyl ether and washed with dilute brine (250 mL), then 2N sodium hydroxide(aq) (2×250 mL) and finally dilute brine (250 mL). The combined organic layers were dried over magnesium sulfate, filtered and concentrated in vacuo. The crude material was purified using silica gel column chromatography eluting with 0% to 100% ethyl acetate in heptanes to afford the title compound (5.61 g, 51%).

¹H NMR (400 MHz, CDCl₃): δ ppm 4.14 (s, 2H), 6.98 (d, 2H), 7.17 (s, 1H), 7.22 to 7.38 (m, 5H), 7.46 (d, 1H), 7.58 (s, 1H), 7.65 (d, 1H).

### Preparation 5

### 5-(benzylthio)-2-fluorobenzonitrile

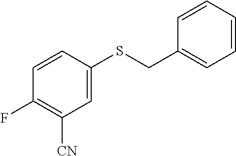 [SMILES: N#Cc1cc(SCc2ccccc2)ccc1F]

To a degassed solution of 5-bromo-2-fluorobenzonitrile (10 g, 50 mmol) in toluene (250 mL) was added N,N-diisopropylethylamine (26 mL, 150 mmol), benzyl mercaptan (5.87 mL, 52 mmol) and dichloro[1,1′ bis(di-tert-butylphosphino)]ferrocene palladium (II) (500 mg, 1 mmol). The reaction mixture was heated at 60° C. for 16 hours. The reaction mixture was diluted to 500 mL volume with ethyl acetate and washed with dilute brine (300 mL), then 2N HCl(aq) (2×300 mL) and finally dilute brine (300 mL). The organic layer was dried over magnesium sulfate, filtered and concentrated in vacuo. The crude material was purified using silica gel column chromatography eluting with 10% ethyl acetate in heptanes to afford the title compound (6.18 g, 51%).

¹H NMR (400 MHz, CDCl₃): δ ppm 4.04 (s, 2H), 7.08 (t, 1H), 7.40 to 7.55 (m, 5H), 7.43 (m, 2H).

### Preparation 6

### 4-[2,4-bis(trifluoromethyl)phenoxy]-3-fluorobenzenesulfonyl chloride

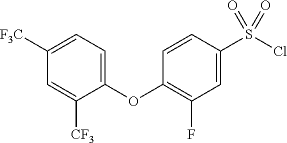 [SMILES: O=S(=O)(Cl)c1ccc(Oc2ccc(C(F)(F)F)cc2C(F)(F)F)c(F)c1]

To a solution of 1-[2,4-bis(trifluoromethyl)phenoxy]-2-fluoro-4-nitrobenzene (Preparation 7, 0.22 g, 0.595 mmol) in EtOH/water (4/1, 2 mL/0.5 mL) was added iron powder (0.166 g, 2.98 mmol) and calcium chloride (0.066 g, 0.595 mmol) and the reaction mixture heated under reflux for 2 hours. The reaction mixture was cooled, filtered through celite and concentrated in vacuo. The residue was diluted with EtOAc, washed with water, brine, dried over Na₂SO₄ and concentrated in vacuo. The crude residue was purified using silica gel column chromatography eluting with 8% EtOAc in heptanes. The resulting aniline was dissolved in concentrated HCl (0.95 mL) at 0° C. A solution of sodium nitrite (0.049 g, 0.713 mmol) in water (0.47 mL) was added and the mixture stirred at 0° C. for 30 minutes. Meanwhile acetic acid (1.7 mL) was saturated with sulphur dioxide at 0° C. and CuCl₂.H₂O was added portionwise. To this solution was added the solution containing sodium nitrite and the reaction mixture was stirred at room temperature for 16 hours. The reaction was diluted with water, extracted with EtOAc (3×40 mL) washed with saturated aqueous NaHCO₃ solution, dried over Na₂SO₄ and concentrated in vacuo. The residue was purified using silica gel column chromatography eluting with 5% EtOAc in heptanes to afford the title compound (140 mg, 48%) that was used without further purification.

### Preparation 7

### 1-[2,4-bis(trifluoromethyl)phenoxy]-2-fluoro-4-nitrobenzene

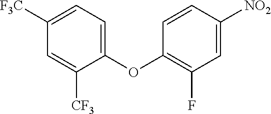 [SMILES: O=[N+]([O-])c1ccc(Oc2ccc(C(F)(F)F)cc2C(F)(F)F)c(F)c1]

The title compound was prepared in a manner analogous to that for the preparation of a compound of formula (I) by Method Variation 4 (as described hereinabove) using 2,4-bis(trifluoromethyl)phenol and 3,4-difluoronitrobenzene. The residue was purified using silica gel column chromatography eluting with 5% EtOAc in Heptanes and used without further purification.

### Preparation 8

### N-(2,4-dimethoxybenzyl)-3,4-difluoro-N-(5-fluoro-1,3-thiazol-2-yl)benzenesulfonamide

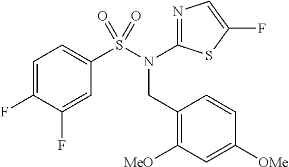 [SMILES: COc1ccc(CN(c2ncc(F)s2)S(=O)(=O)c2ccc(F)c(F)c2)c(OC)c1]

A suspension of 3,4-difluoro-N-(5-fluoro-1,3-thiazol-2-yl)benzenesulfonamide (Preparation 9, 150 mg, 0.51 mmol), 2,4-dimethoxybenzylalcohol (91 mg, 0.541 mmol) and triphenylphosphine (140 mg, 0.534 mmol) in THF (5 mL) was cooled to 0° C. DIAD (0.1 mL, 0.508 mmol) was added and the reaction stirred for 4 hours. The reaction was quenched by the addition of saturated aqueous sodium chloride solution (10 mL), extracted into EtOAc (10 mL), dried over sodium sulfate and concentrated in vacuo. The residue was purified using silica gel column chromatography eluting with 20% EtOAc in heptane to afford the title compound.

¹H NMR (400 MHz, DMSO-d₆): δ ppm 3.65 (s, 3H), 3.70 (s, 3H), 4.85 (s, 2H), 6.40 (d, 1H), 6.45 (s, 1H), 7.05 (d, 1H), 7.40 (d, 1H), 7.70 (m, 2H), 7.95 (t, 1H).

MS m/z 467 [M+Na]⁺

### Preparation 9

### 3,4-difluoro-N-(5-fluoro-1,3-thiazol-2-yl)benzenesulfonamide

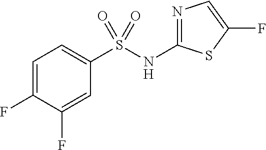 [SMILES: O=S(=O)(Nc1ncc(F)s1)c1ccc(F)c(F)c1]

A solution of 3,4-difluoro-N-(1,3-thiazol-2-yl)benzenesulfonamide (WO2012004743, 1.99 g, 7.02 mmol) and Selectfluor™ (3.12 g, 8.81 mmol) in MeCN/water (25 mL/5 mL) was stirred at 45° C. under nitrogen for 18 hours. The mixture was filtered to furnish a white solid as the fluorohydrin, which was suspended in DCM (15 mL) and treated with triethylamine (6.2 mL, 44.5 mmol) followed by acetic anhydride (1.3 mL). The reaction was stirred at room temperature for 18 hours. The reaction was washed with 2 M HCl (aq) (50 mL), dried over Na₂SO₄ and concentrated in vacuo. The residue was triturated with DCM to afford the title compound.

¹H NMR (400 MHz, DMSO-d₆): δ ppm 7.40 (s, 1H), 7.55-7.65 (m, 2H), 7.80 (t, 1H).

MS m/z 295 [M+H]⁺

### Preparation 10

### 3-bromo-4,5-difluoro-N-(1,2,4-thiadiazol-5-yl)benzenesulfonamide

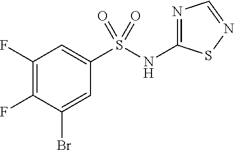 [SMILES: O=S(=O)(Nc1ncns1)c1cc(F)c(F)c(Br)c1]

To an ice-cooled (0° C.) solution of 5-amino-1,2,4-thiadiazole (758 mg, 7.5 mmol) in dioxane (15 mL) was added NaOH (300 mg, 7.5 mmol) and water (2.5 mL). 3,4-Difluoro-5-bromobenzenesulfonyl chloride (875 mg, 3.0 mmol) was added and the reaction mixture stirred at 0° C. for 1 hour. The reaction was quenched by the addition of 2M HCl (aq) and extracted into DCM (3×20 mL). The organic layers were collected, combined, dried over MgSO₄ and concentrated in vacuo. The resulting solid was washed with 1M HCl (aq) (50 mL), water, and dried to afford the title compound (752 mg, 70%).

¹H NMR (400 MHz, DMSO-d₆): δ ppm 7.88-7.95 (m, 2H), 8.52 (s, 1H).

MS m/z 358 [M+H]⁺

### Preparation 11

### 3-chloro-4-fluoro-N-(1,2,4-thiadiazol-5-yl)benzenesulfonamide

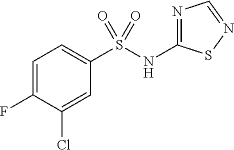 [SMILES: O=S(=O)(Nc1ncns1)c1ccc(F)c(Cl)c1]

To a solution of 3-chloro-4-fluoro-benzenesulfonyl chloride (6 g, 26.2 mmol) in THF (30 mL) was added triethylamine (8.1 mL, 58 mmol) followed by 5-amino-1,2,4-thiadiazole (2.91 g, 28.8 mmol) and the reaction mixture was stirred at room temperature for 18 hours. The reaction was quenched by the addition of 2N HCl (60 mL), water (120 mL) and stirred for 2 hours. The supernantant liquor was decanted, and the resulting gum extracted into EtOAc (50 mL), washed with water (50 mL), brine (50 mL), dried and decolourised over MgSO₄ and charcoal, and concentrated in vacuo. The residue was triturated with TBME (2×5 mL) to afford the title compound as the desired product (3.6 g, 47%).

¹H NMR (400 MHz, DMSO-d₆): δ ppm 7.60 (t, 1H), 7.83 (m, 1H), 7.96 (m, 1H), 8.49 (s, 1H).

### Preparation 12

### 2,4,5-trifluoro-N-(prop-2-en-1-yl)-N-(1,3-thiazol-2-yl)benzenesulfonamide

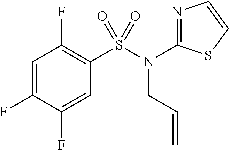 [SMILES: C=CCN(c1nccs1)S(=O)(=O)c1cc(F)c(F)cc1F]

To a solution of 2,4,5-trifluoro-N-(1,3-thiazol-2-yl)benzenesulfonamide (Preparation 36, 300 mg, 1.02 mmol) and potassium carbonate (223 mg, 1.40 mmol) in THF (30 mL) was added allyl bromide (0.113 mL, 1.30 mmol) and the reaction mixture heated to 70° C. for 18 hours. The reaction was cooled and concentrated in vacuo. The residue was partitioned between DCM (100 mL) and water (50 mL), the organic layer collected, dried over MgSO₄ and concentrated in vacuo. The residue was purified using silica gel column chromatography eluting with 1:1 EtOAc:cyclohexane to afford the title compound.

¹H NMR (400 MHz, CDCl₃): δ ppm 4.60 (d, 2H), 5.10-5.30 (m, 2H), 5.80-5.90 (m, 1H), 6.60 (m, 1H), 6.90 (m, 1H), 7.00 (m, 1H), 7.80-7.90 (m, 1H).

### Preparation 13

### 2,4,5-trifluoro-N-(5-fluoro-1,3-thiazol-2-yl)-N-(prop-2-en-1-yl)benzenesulfonamide

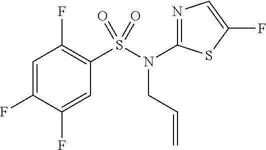 [SMILES: C=CCN(c1ncc(F)s1)S(=O)(=O)c1cc(F)c(F)cc1F]

The title compound was prepared according to the procedure described in Preparation 12 using 2,4,5-trifluoro-N-(5-fluoro-1,3-thiazol-2-yl)benzenesulfonamide (Preparation 14).

¹H NMR (400 MHz, CDCl₃): δ ppm 4.50 (d, 2H), 5.20-5.35 (m, 2H), 5.80 (m, 1H), 6.60 (m, 1H), 7.00 (m, 1H), 7.80-7.90 (m, 1H).

### Preparation 14

### 2,4,5-trifluoro-N-(5-fluoro-1,3-thiazol-2-yl)benzenesulfonamide

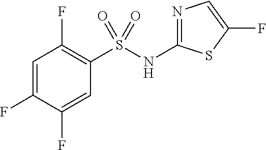 [SMILES: O=S(=O)(Nc1ncc(F)s1)c1cc(F)c(F)cc1F]

The title compound was prepared according to the procedure described in Preparation 9 using 2,4,5-trifluoro-N-(1,3-thiazol-2-yl)benzenesulfonamide (Preparation 15). ¹H NMR (400 MHz, DMSO-d₆): δ ppm 7.40 (m, 1H), 7.80-7.90 (m, 2H), 12.90 (br s, 1H).

### Preparation 15

### 2,4,5-trifluoro-N-(1,3-thiazol-2-yl)benzenesulfonamide

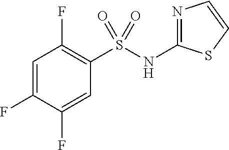 [SMILES: O=S(=O)(Nc1nccs1)c1cc(F)c(F)cc1F]

The title compound was prepared according to the procedure described in Preparation 18 using 2,4,5-trifluorobenzenesulfonyl chloride and 2-aminothiazole. Taken directly on to the next step.

### Preparation 16

### 4,5-trifluoro-N-(5-fluoro-1,3-thiazol-2-yl)-N-(prop-2-en-1-yl)benzenesulfonamide

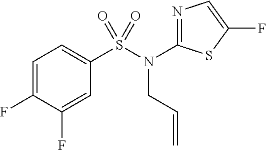 [SMILES: C=CCN(c1ncc(F)s1)S(=O)(=O)c1ccc(F)c(F)c1]

The title compound was prepared according to the procedure described in Preparation 12 using 3,4-difluoro-N-(5-fluoro-1,3-thiazol-2-yl)benzenesulfonamide (Preparation 9).

¹H NMR (400 MHz, DMSO-d₆): δ ppm 4.50 (m, 2H), 5.00 (m, 1H), 5.15 (m, 1H), 5.80 (m, 1H), 7.50 (s, 1H), 7.60-7.70 (m, 2H), 7.90 (m, 1H).

### Preparation 17

### 3,4-difluoro-N-(prop-2-en-1-yl)-N-(1,3-thiazol-2-yl)benzenesulfonamide

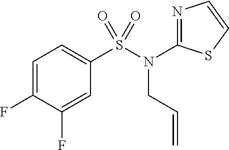 [SMILES: C=CCN(c1nccs1)S(=O)(=O)c1ccc(F)c(F)c1]

The title compound was prepared according to the procedure described in Preparation 12 using 3,4-difluoro-N-(1,3-thiazol-2-yl)benzenesulfonamide (WO2010079443).

¹H NMR (400 MHz, DMSO-d₆): δ ppm 4.60 (m, 2H), 4.90 (m, 1H), 5.20 (m, 1H), 5.90 (m, 1H), 7.10 (m, 1H), 7.40 (m, 1H), 7.50-7.65 (m, 2H), 7.80 (m, 1H).

### Preparation 18

### 2,4-difluoro-N-(thiazol-2-yl)benzenesulfonamide

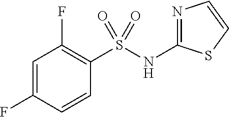 [SMILES: O=S(=O)(Nc1nccs1)c1ccc(F)cc1F]

To a slurry of 2-aminothiazole (15.08 g, 0.1506 mol) in methylene chloride (100 mL) and pyridine (24 mL, 0.30 mol) was added dropwise over 20 minutes a solution of 2,4-difluorobenzenesulfonyl chloride (10 mL, 0.07 mol) in 10 mL of methylene chloride. After stirring at room temperature for 48 hours the reaction mixture was concentrated and purified by silica gel column chromatography eluting with hexane/ethyl acetate to afford the title compound.

MS m/z 277 [M+H]⁺

The following Preparations were prepared according to the procedure described in Preparation 37 using N-(2,4-dimethoxybenzyl)-1,3,4-thiadiazol-2-amine (WO2012004743), N-(2,4-dimethoxybenzyl)-1,2,4-thiadiazol-5-amine (WO2012004743), N-(2,4-dimethoxybenzyl)-1,3-thiazol-2-ylamine (WO2012004743) or thiazole-4-yl-carbamic acid tert-butyl ester (WO2012004706), with either LiHMDS or NaHMDS as base, and the appropriate arylsulfonylchloride as described below:

| | | Arylsulfonyl | |
|---|---|---|---|
| Prep | Name | chloride | Data |
| 19 | N-(2,4- | 3,4,5- | m/z 445 [M]⁺ |
| | dimethoxybenzyl)- | trifluorobenzenesulfonyl | |
| | 3,4,5-trifluoro-N-(1,2,4- | chloride | |
| | thiadiazol-5- | | |
| | yl)benzenesulfonamide | | |
| 20 | N-(2,4- | 2,4-difluoro-5- | ¹H NMR (400 MHz, CDCl₃): δ |
| | dimethoxybenzyl)-2,4- | methylbenzenesulfonyl | ppm 3.70 (s, 3H), 3.75 (s, 3H), |
| | difluoro-5-methyl-N- | chloride | 5.30 (s, 2H), 6.35 (m, 1H), |
| | (1,3,4-thiadiazol-2- | (WO2005118529) | 6.80-6.85 (m, 1H), 7.20-7.25 (m, 2H), |
| | yl)benzenesulfonamide | | 8.60 (t, 1H), 8.80 (s, 1H). |
| 21 | N-(2,4- | 3,4- | ¹H NMR (CDCl₃): δ ppm 3.60 (s, |
| | dimethoxybenzyl)-3,4- | difluorobenzenesulfonyl | 3H), 3.65 (s, 3H), 5.20 (s, 2H), |
| | difluoro-N-(1,2,4- | chloride | 6.25-6.30 (m, 2H), 7.00 (m, 1H), |
| | thiadiazol-5- | | 7.10-7.20 (m, 1H), 7.40-7.55 (m, |
| | yl)benzenesulfonamide | | 2H), 8.10 (s, 1H). |
| 22 | N-(2,4- | 4-fluoro-5- | ¹H NMR (400 MHz, DMSO-d₆): δ |
| | dimethoxybenzyl)-4- | methylbenzensulfonyl | ppm 2.23 (s, 3H), 3.71 (2 × s, |
| | fluoro-3-methyl-N- | chloride | 6H), 6.40-6.47 (m, 2H), 6.97 (m, |
| | (1,2,4-thiadiazol-5- | | 1H), 7.40 (m, 1H), 7.78 (m, 2H), |
| | yl)benzenesulfonamide | | 8.38 (s, 1H). |
| 23 | N-(2,4- | 2,4,5- | ¹H NMR (400 MHz, CDCl₃): δ |
| | dimethoxybenzyl)- | trifluorobenzenesulfonyl | ppm 3.75 (s, 3H), 3.78 (s, 3H), |
| | 2,4,5-trifluoro-N-(1,3,4- | chloride | 5.30 (s, 2H), 6.30 (m, 1H), |
| | thiadiazol-2- | | 6.35 (m, 1H), 7.00 (m, 1H), 7.20 (m, |
| | yl)benzenesulfonamide | | 1H), 7.65 (m, 1H), 8.80 (s, 1H). |
| 24 | tert-butyl [(4-fluoro-3- | 4-fluoro-3- | ¹H NMR (400 MHz, DMSO-d₆): δ |
| | iodophenyl)sulfonyl]1,3- | iodobenzenesulfonyl | ppm 1.27 (s, 9H), 7.58-7.65 (m, |
| | thiazol-4-ylcarbamate | chloride | 1H), 8.09-8.15 (m, 2H), |
| | | | 8.44 (dd, 1H), 9.17 (d, 1H). |
| 25 | tert-butyl [(2-chloro-4- | 2,4-difluoro-5- | ¹H NMR (400 MHz, CDCl₃): δ |
| | fluorophenyl)sulfonyl]1, | chlorobenzenesulfonyl | ppm 1.35 (s, 9H), 7.15 (m, 1H), |
| | 3-thiazol-4- | chloride | 7.25 (m, 1H), 7.55 (s, 1H), |
| | ylcarbamate | | 8.37 (m, 1H), 8.80 (s, 1H). |
| 26 | tert-butyl [(2,4-difluoro- | 2,4-difluoro-5- | ¹H NMR (400 MHz, CDCl₃): δ |
| | 5- | methylbenzenesulfonyl | ppm 1.25 (s, 9H), 2.36 (s, 3H), |
| | methylphenyl)sulfonyl]1, | chloride | 6.94 (t, 1H), 7.53 (s, 1H), 8.00 (t, |
| | 3-thiazol-4- | (WO2005118529) | 1H), 8.79 (s, 1H). |
| | ylcarbamate | | |
| 27 | tert-butyl [(2,4- | 2,4- | ¹H NMR (400 MHz, CDCl₃): δ |
| | difluorophenyl)sulfonyl]1, | difluorobenzenesulfonyl | ppm 1.35 (s, 9H), 6.92-7.02 (m, |
| | 3-thiazol-4- | chloride | 1H), 7.04-7.09 (m, 1H), 7.53 (s, |
| | ylcarbamate | | 1H), 8.12-8.22 (m, 1H), 8.80 (s, |
| | | | 1H). |
| 28 | tert-butyl [(4-fluoro-2- | 2-methoxy-4- | ¹H NMR (400 MHz, CDCl₃): δ |
| | methoxyphenyl)sulfonyl]1, | fluorobenzenesulfonyl | ppm 1.35 (s, 9H), 3.90 (s, 3H), |
| | 3-thiazol-4- | chloride | 6.80 (m, 2H), 7.50 (m, 1H), |
| | ylcarbamate | | 8.15 (m, 1H), 8.80 (s, 1H). |
| 29 | N-(2,4- | 2-methoxy-4- | ¹H NMR (400 MHz, CDCl₃): δ |
| | dimethoxybenzyl)-4- | fluorobenzenesulfonyl | ppm 3.65 (s, 3H), 3.75 (s, 6H), |
| | fluoro-2-methoxy-N- | chloride | 5.20 (s, 2H), 6.35 (m, 2H), |
| | (1,3,4-thiadiazol-2- | | 6.60 (m, 1H), 6.70-6.75 (m, 1H), |
| | yl)benzenesulfonamide | | 7.25 (m, 1H), 7.95 (m, 1H), 8.90 (m, |
| | | | 1H). |
| 30 | tert-butyl [(4-fluoro-2- | 2-chloro-4- | Used without further purification. |
| | chloro- | fluorobenzenesulfonyl | |
| | phenyl)sulfonyl]1,3- | chloride | |
| | thiazol-4-ylcarbamate | | |
| 31 | N-(2,4- | 2-chloro-4- | Used without further purification. |
| | dimethoxybenzyl)-4- | fluorobenzene | |
| | fluoro-2-chloro-N- | sulfonyl chloride | |
| | (1,3,4-thiadiazol-2- | | |
| | yl)benzenesulfonamide | | |
| 32 | N-(2,4- | 3,4- | Used without further purification. |
| | dimethoxybenzyl)-3,4- | difluorobenzenesulfonyl | |
| | difluoro-N-(1,3-thiazol- | chloride | |
| | 2-yl)- | | |
| | benzenesulfonamide | | |

### Preparation 33

### 3-cyano-4-fluoro-N-(3-methylisoxazol-4-yl)benzenesulfonamide

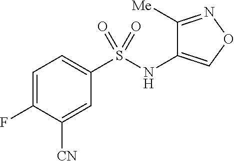 [SMILES: Cc1nocc1NS(=O)(=O)c1ccc(F)c(C#N)c1]

To a suspension of 3-methylisoxazol-4-ylamine (140 mg, 1.04 mmol) in DCM (5 mL) and pyridine (0.252 uL, 3.12 mmol) was added 4-fluoro-3-cyanobenzene sulfonylchloride (275 mg, 1.25 mmol) and the reaction mixture stirred at room temperature for 18 hours. The reaction mixture was washed with water, the organic layer collected, dried over MgSO₄ and concentrated in vacuo. The residue was re-dissolved in DCM, washed with 2N HCl (aq), the organic layer collected, dried over MgSO₄ and concentrated in vacuo to afford the title compound as a yellow solid (196 mg, 67%), which was used without further purification

The following Preparations were prepared according to the procedure described in Preparation 33 using the appropriate arylsulfonylchloride and aminoheterocycle as described below:

| | | Arylsulfonyl chloride | |
|---|---|---|---|
| Prep | Name | and aminoheterocyle | Data |
| 34 | 3-cyano-4-fluoro-N-(1- | 3-cyano-4- | MS m/z 279 [M − H]⁻ |
| | methyl-1H-pyrazol-4- | fluorobenzenesulfonyl | |
| | yl)benzenesulfonamide | chloride and 1-methyl- | |
| | | 1H-pyrazol-4-ylamine | |
| 35 | 3-cyano-4-fluoro-N-(3- | 3-cyano-4- | ¹H NMR (400 MHz, |
| | methyl-1,2-oxazol-5- | fluorobenzenesulfonyl | CD₃OD): δ ppm 2.19 (s, |
| | yl)benzenesulfonamide | chloride and 3-methyl- | 3H), 7.60 (t, 1H), 7.68 (dd, |
| | | 1,2-oxazol-5-ylamine | 1H), 8.25 (m, 1H), |
| | | | 8.35 (dd, 1H), 8.68 (s, 1H). |
| 36 | 2,4,5-trifluoro-N-(1,3- | 2,4,5- | ¹H NMR (400 MHz, DMSO- |
| | thiazol-2- | trifluorobenzenesulfonyl | d₆): δ ppm 6.90 (m, 1H), |
| | yl)benzenesulfonamide | chloride and 2- | 7.30 (m, 1H), 7.75-7.90 (m, |
| | | aminothiazole | 2H), 13.0 (br s, 1H). |
| 37 | 2-chloro-4-fluoro-N- | 2-chloro-4- | ¹H NMR (400 MHz, DMSO- |
| | (1,3,4-thiadiazol-2- | fluorobenzenesulfonyl | d₆): δ ppm 7.44 (m, 1H), |
| | yl)benzenesulfonamide | chloride and 2-amino- | 7.68 (m, 1H), 8.11 (m, 1H), |
| | | 1,3,4-thiadiazole | 8.81 (s, 1H). |

### Preparation 38

### Tert-butyl[(5-bromo-2,4-difluorophenyl)sulfonyl]1,3-thiazol-4-ylcarbamate

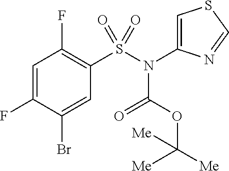 [SMILES: CC(C)(C)OC(=O)N(c1cscn1)S(=O)(=O)c1cc(Br)c(F)cc1F]

To a solution of thiazole-4-yl-carbamic acid tert-butyl ester (WO201004707, 1650 mg, 8.23 mmol) in THF (29.3 mL) was added LiHMDS (8.23 mL, 8.23 mmol) at 0° C. After stirring for 1 hour at this temperature, the reaction mixture was cooled to −78° C. and 2,4-difluoro-5-bromobenzenesulfonamide (2000 mg, 6.86 mmol) in THF (5.0 mL) was added. The mixture was allowed to warm to room temperature over 18 hours. The reaction was quenched by the addition of saturated aqueous ammonium chloride solution (60 mL) and extracted into DCM. The organic layer was collected, dried over MgSO₄ and concentrated in vacuo. The residue was dissolved in DCM (10 mL), TFA (10 mL) was added and the reaction mixture stirred at room temperature for 18 hours. The reaction mixture was concentrated in vacuo and purified using silica gel column chromatography eluting with 50:50 EtOAc:Heptane to afford the title compound as a white solid (2.08 g, 85%).

¹H NMR (400 MHz, DMSO-d₆): δ ppm 1.35 (s, 9H), 7.10 (s, 1H), 7.75 (m, 1H), 8.10 (m, 1H), 8.90 (m, 1H), 11.45 (br s, 1H).

### Preparation 39

### tert-butyl-3-fluoro-4-hydroxy-N-(thiazol-2-yl)benzenesulfonamide

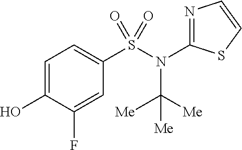 [SMILES: CC(C)(C)N(c1nccs1)S(=O)(=O)c1ccc(O)c(F)c1]

The title compound was prepared in a manner analogous to that for the preparation of a compound of formula (I) by Method Variation 10 (as described hereinabove) at 0° C. using trimethylsilylethanol and tert-butyl [(3,4-difluorophenyl)sulfonyl]1,3-thiazol-2-ylcarbamate (WO2010079443) and isolated as a white solid.

¹H NMR (400 MHz, DMSO-d₆): δ ppm 1.35 (s, 9H), 7.16 (m, 1H), 7.60-7.85 (m, 4H), 11.17 (br s, 1H).

### Preparation 40

### 4-cyano-2-(difluoromethoxy)phenol

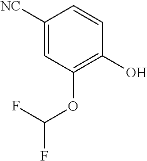 [SMILES: N#Cc1ccc(O)c(OC(F)F)c1]

The title compound was prepared according to the method described for Preparation 41 using 4-cyano-2-(difluoromethoxy)fluorobenzene (Preparation 42).

MS m/z 184 [M−H]⁺

### Preparation 41

### 5-cyano-2-(difluoromethoxy)phenol

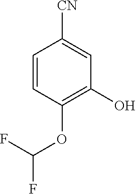 [SMILES: N#Cc1ccc(OC(F)F)c(O)c1]

To a solution of trimethylsilylethanol (0.953 mL, 6.68 mmol) in THF (20 mL) was added sodium hydride (267 mg, 6.68 mmol) at 0° C. The reaction mixture was stirred at this temperature for 30 minutes before the addition of 5-cyano-2-(difluoromethoxy)fluorobenzene (Preparation 43, 500 mg, 2.67 mmol). The reaction mixture was stirred at room temperature for 5 hours. The reaction was quenched by the addition of methanol and concentrated in vacuo. The residue was purified using silica gel column chromatography eluting with 10-80% EtOAc in heptanes. The residue was dissolved in THF (6 mL) and TBAF (2 mL) was added. The reaction mixture was stirred at room temperature for 5 hours before the addition of silica gel and concentrating in vacuo. The residue was purified using silica gel column chromatography eluting with 10-100% EtOAc in heptanes to afford the title compound (260 mg, 41%).

MS m/z 184 [M−H]⁺

### Preparation 42

### 4-cyano-2-(difluoromethoxy)fluorobenzene

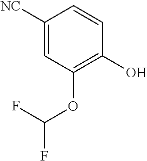 [SMILES: N#Cc1ccc(O)c(OC(F)F)c1]

The title compound was prepared according to the method described for Preparation 47 using 2-fluoro-5-cyanophenol. Taken directly on to the next step.

### Preparation 43

### 5-cyano-2-(difluoromethoxy)fluorobenzene

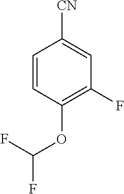 [SMILES: N#Cc1ccc(OC(F)F)c(F)c1]

The title compound was prepared according to the method described for Preparation 47 using 2-fluoro-4-cyanophenol.

¹H NMR (400 MHz, DMSO-d₆): δ ppm 7.30 (t, 1H), 7.60 (m, 1H), 7.85 (m, 1H), 8.00 (m, 1H).

### Preparation 44

### 4-chloro-2-(difluoromethoxy)phenol

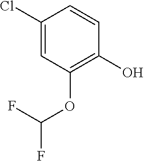 [SMILES: Oc1ccc(Cl)cc1OC(F)F]

The title compound was prepared according to the method described for Preparation 45 using 4-chloro-2-(difluoromethoxy)bromobenzene (Preparation 46).

MS m/z 193 [M−H]⁻

### Preparation 45

### 5-chloro-2-(difluoromethoxy)phenol

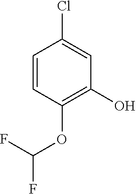 [SMILES: Oc1cc(Cl)ccc1OC(F)F]

To a solution of 5-chloro-2-(difluoromethoxy)bromobenzene (Preparation 46, 200 mg, 0.63 mmol) in THF (6 mL) was added bis-neopentylglycolatodiboron (149 mg, 0.660 mmol), potassium acetate (191 mg, 1.88 mmol) and Pd(dppf)Cl₂ (23 mg, 0.031 mmol). The reaction was heated under reflux for 4 hours before cooling and diluting with EtOAc and water. The organic layer was collected, washed with brine, dried over Na₂SO₄ and concentrated in vacuo. The residue was dissolved in acetone (6 mL) and treated with a solution of oxone (1.64 g, 2.54 mmol) in water (6 mL). The reaction was stirred at room temperature for 15 minutes. The reaction was diluted with diethyl ether (10 mL) and water (5 mL). The organic layer was collected, washed with brine, dried over Na₂SO₄ and concentrated in vacuo. The residue was purified using silica gel column chromatography eluting with 0-15% diethyl ether in DCM to afford the title compound (quant).

MS m/z 193 [M−H]⁻

### Preparation 46

### 4-chloro-2-(difluoromethoxy)bromobenzene

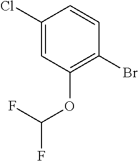 [SMILES: FC(F)Oc1cc(Cl)ccc1Br]

The title compound was prepared according to the method described for Preparation 47 using 2-bromo-5-chlorophenol and used without further purification.

### Preparation 47

### 5-chloro-2-(difluoromethoxy)bromobenzene

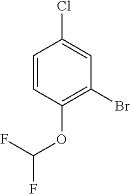 [SMILES: FC(F)Oc1ccc(Cl)cc1Br]

To a solution of 2-bromo-4-chlorophenol (1 g, 4.8 mmol) in DMF/water (45 mL/5 mL) was added sodium chlorodifluoroacetate (1.95 g, 12.0 mmol) and cesium carbonate (3.14 g, 9.64 mmol), and the reaction mixture was heated to 100° C. for 4 hours. The reaction mixture was cooled and diluted with TBME (20 mL) and water (20 mL). The organic layer was collected, washed with saturated aqueous NaHCO₃ solution, brine, dried over Na₂SO₄ and concentrated in vacuo to afford the title compound (1.21 g, 98%).

¹HNMR (400 MHz, CDCl₃): δ ppm 6.52 (m, 1H), 7.18 (t, 1H), 7.31 (m, 1H), 7.64 (d, 1H).

### Preparation 48

### 4-chloro-2-cyclobutyloxyphenol

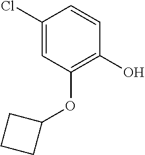 [SMILES: Oc1ccc(Cl)cc1OC1CCC1]

To a solution of 3-chloro-6-methoxyphenol (250 mg, 1.58 mmol), cyclobutanol (114 mg, 1.58 mmol) and triphenylphosphine (496 mg, 1.89 mmol) in THF (4 mL) was added a solution of DIAD (407 mg, 1.89 mmol) in THF (4 mL) at 0° C. The reaction mixture was stirred at room temperature for 18 hours. The reaction mixture was diluted with EtOAc (20 mL) and washed with saturated aqueous NaHCO₃ solution (2×15 mL), brine, dried over Na₂SO₄ and concentrated in vacuo. The residue was purified using silica gel column chromatography eluting with 0-30% EtOAc in heptanes. The residue was added to a solution of 2-diethylaminoethanethiol (268 mg, 1.58 mmol) and sodium tert-butoxide (326 mg, 3.29 mmol) that had stirred together for 15 minutes. The reaction mixture was heated under reflux for 1 hour before cooling to 0° C. The reaction was quenched by the addition of 1N HCl (aq) to pH=1 and extracted into EtOAc, washed with brine, dried over Na₂SO₄ and concentrated in vacuo. The residue was purified using silica gel column chromatography eluting with 0-30% EtOAc in heptanes to afford the title compound. (62 mg, 48%).

MS m/z 197 [M−H]⁻

### Preparation 49

### 4-chloro-2-D₃-methoxyphenol

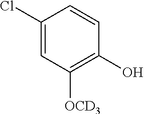

To a solution of 3-D₃-methoxy-4-acetylchlorobenzene (Preparation 50, 200 mg, 1.07 mmol) in DCM (10 mL) was added metachloroperbenzoic acid (276 mg, 1.6 mmol) and TFA (200 uL) and the reaction mixture was stirred at reflux for 18 hours. The cooled reaction mixture was poured onto 10% aqueous sodium metabisulphite (100 mL) and extracted with DCM (3×50 mL). The combined organic extracts were washed with saturated aqueous NaHCO₃ (3×100 mL), dried over MgSO₄ and concentrated in vacuo. The residue was dissolved in THF/water and sodium hydroxide (40 mg, 1 mmol) was added and the reaction stirred at room temperature for 18 hours. The reaction mixture was concentrated in vacuo then partitioned between EtOAc and water. The aqueous layer was collected and acidified with 2N HCl (aq). The aqueous was extracted with EtOAc, dried over MgSO₄ and concentrated in vacuo to afford the title compound as an oil (125 mg, 78%), which was used without further purification.

### Preparation 50

### 3-D₃-methoxy-4-acetylchlorobenzene

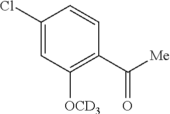

A solution of 3-chloro-4-acetylphenol (5 g, 5.88 mmol) in DMF (5 mL) was added potassium carbonate (974 mg, 7.05 mmol) followed by CD3I (1.02 g, 7.05 mmol) and the reaction mixture was stirred at 50° C. for 18 hours. The reaction mixture was poured into diethyl ether and washed with water, dried over MgSO₄ and concentrated in vacuo to afford the title compound as a pale yellow solid (1.24 g, 94%).

MS m/z 188 [M+H]⁺

### Preparation 51

### 3-cyano-4-fluoro-N-1,3,4-thiadiazol-2-yl-benzenesulfonamide

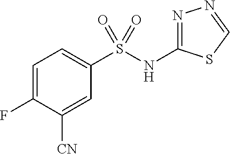 [SMILES: N#Cc1cc(S(=O)(=O)Nc2nncs2)ccc1F]

3-cyano-4-fluoro-benzenesulfonylchloride (169 g, 0.77 mol) was added portionwise over 1 hour to a stirred suspension of 2-amino-1,3,4-thiadiazole (78 g, 0.77 mol) and pyridine (64.6 mL, 1.54 mol) in DCM (1000 mL). The reaction mixture was stirred at room temperature for 72 hours. The reaction mixture was concentrated in vacuo and triturated with water (2×250 mL). The resulting solid was further triturated with 3:1 TBME:acetone (3×100 mL) before being treated with saturated aqueous sodium carbonate solution (1000 mL). The aqueous solution was washed with EtOAc (2×200 mL) and acidified with 1M HCl to pH=6-7. The resulting precipitate was collected by filtration and washed with water to afford the title compound as a brown powder (45 g, 21%).

¹HNMR (400 MHz, DMSO-d₆): δ ppm 7.65 (m, 1H), 8.15 (m, 1H), 8.35 (m, 1H), 8.80 (s, 1H).

### Preparation 52

### 4-(3-chloro-4-formylphenoxy)-3-cyano-N-(1,3,4-thiadiazol-2-yl)benzenesulfonamide

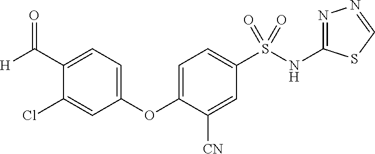 [SMILES: N#Cc1cc(S(=O)(=O)Nc2nncs2)ccc1Oc1ccc(C=O)c(Cl)c1]

To a solution of 3-cyano-4-fluoro-N-(1,3,4-thiadiazol-2-yl)benzenesulfonamide (Preparation 51, 200 mg, 0.70 mmol) in DMSO (3.5 mL) was added 2-chloro-4-hydroxybenzaldehyde (132 mg, 0.84 mmol) and potassium phosphate dibasic (368 mg, 2.11 mmol). The reaction mixture was heated to 100° C. for 20 hours. The reaction mixture was cooled to room temperature and diluted with water (10 mL) and 1M HCl (20 mL). The mixture was extracted with EtOAc (3×50 mL) and the combined organic layers washed with brine (100 mL). The organic layer was dried over MgSO₄ and concentrated in vacuo. The residue was purified by silica gel column chromatography eluting with 20-50% EtOAc in heptanes followed by trituration with TBME to afford the title compound as a white solid (90 mg, 31%).

¹HNMR (400 MHz, DMSO-d₆): δ ppm 7.29 (d, 1H), 7.36 (dd, 1H), 7.61 (d, 1H), 7.95 (d, 1H), 8.06 (dd, 1H), 8.31 (d, 1H), 8.80 (s, 1H), 10.27 (s, 1H).

### Biological Assay

### 1. Generation of a Custom Clonal Cell Line for URAT1 Transporter Activity Assay

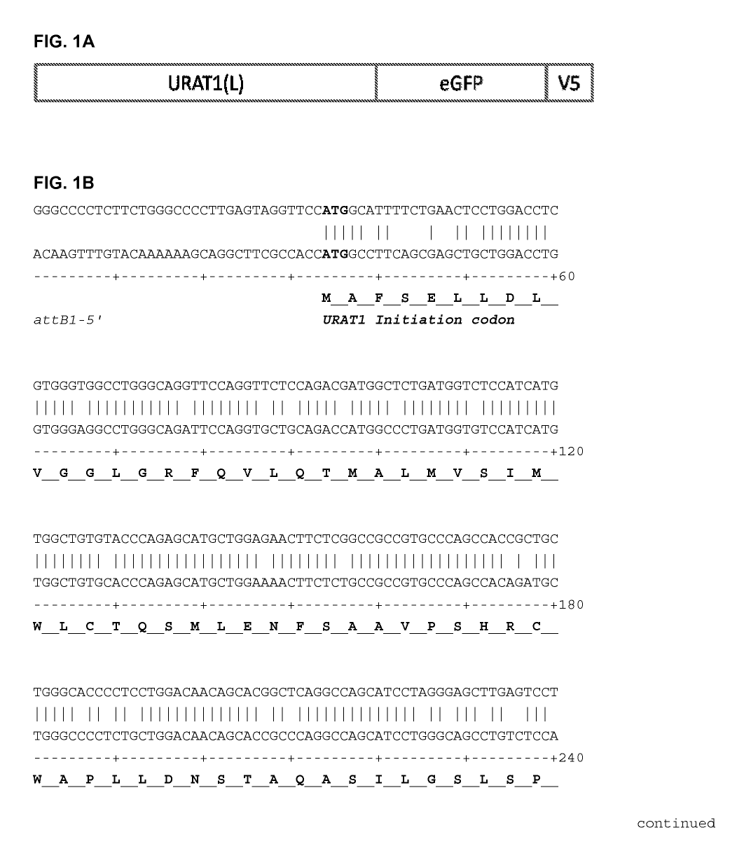

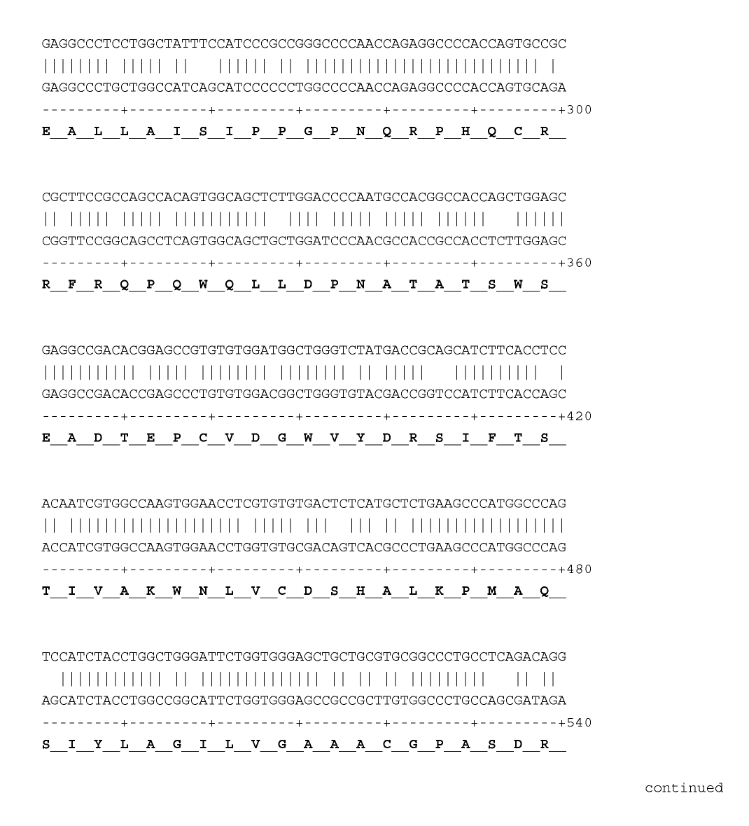

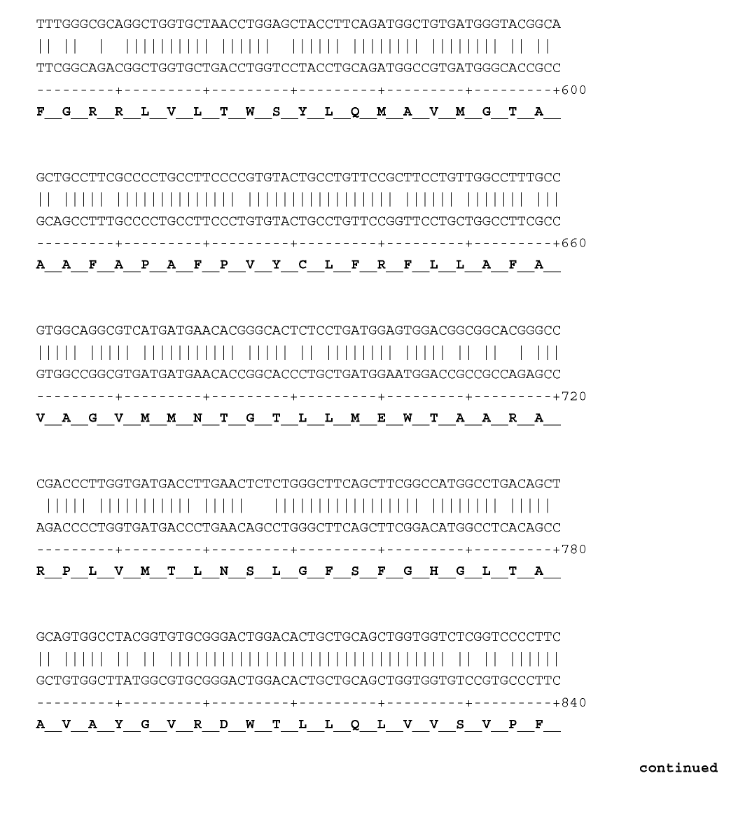

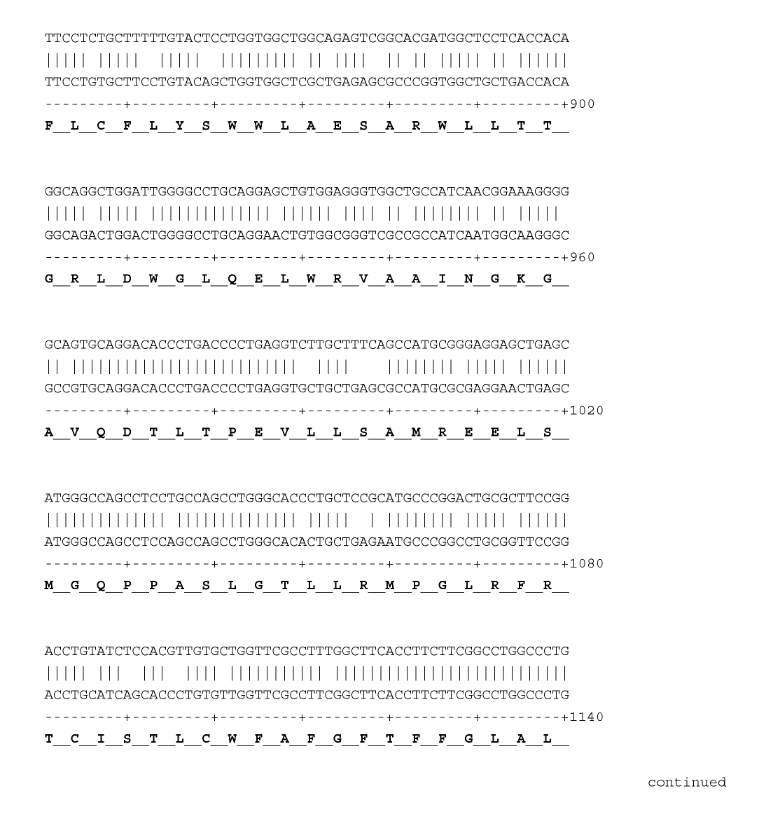

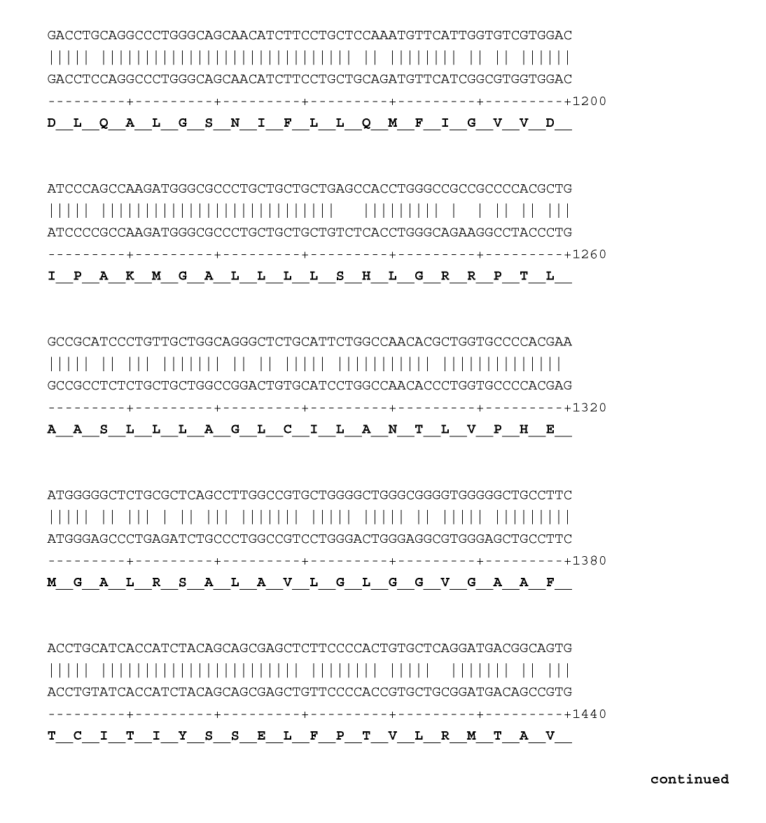

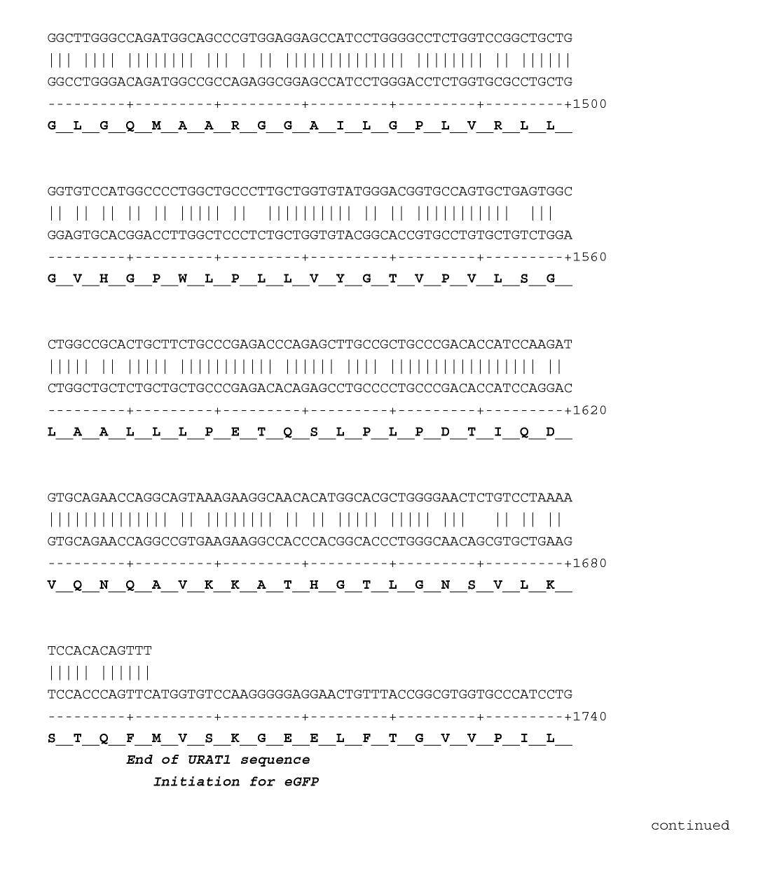

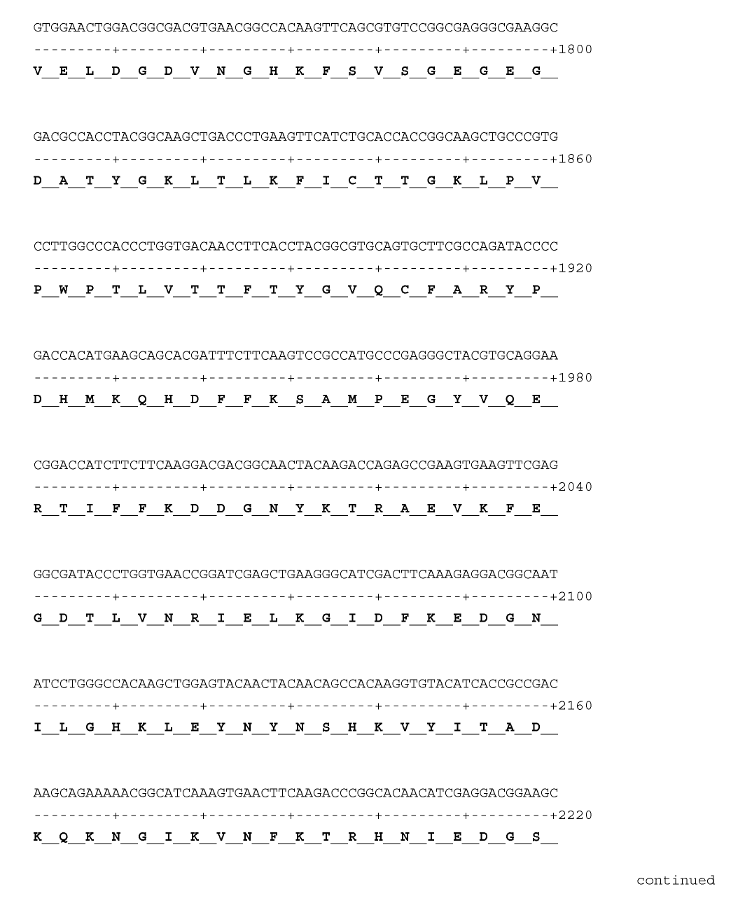

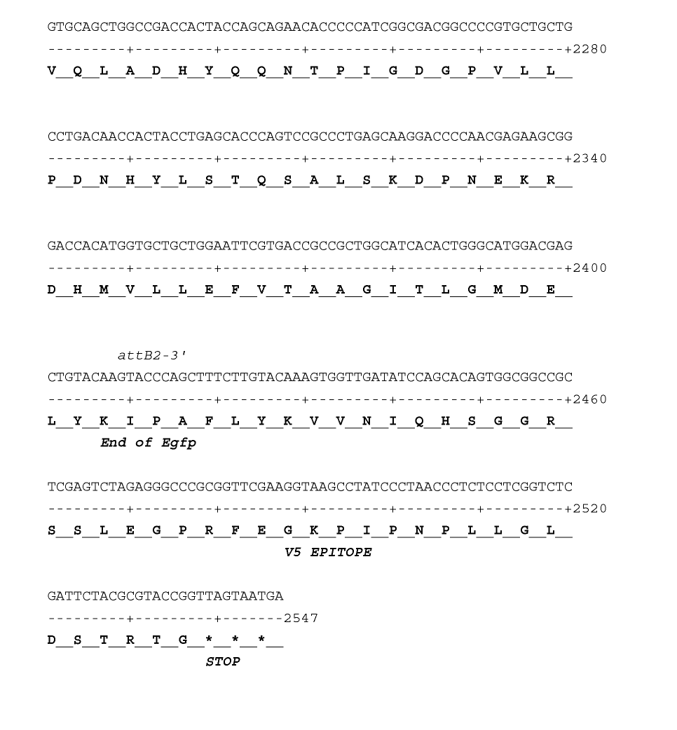

The nucleotide sequence for the long isoform of URAT1 (NM_{—}144585) was C-terminally fused to that of enhanced green fluorescent protein (eGFP) (hereinafter referred to as URAT1(L)GFP). The combined sequence was codon-optimised and custom synthesized. The synthesized sequence was generated in pDONR221 Gateway entry vector (Invitrogen Life Technologies) prior to cloning in pLenti6.3/V5 Gateway destination vector (Invitrogen Life Technologies). A schematic of the URAT1(L)GFP construct is set forth in FIG. 1A. The nucleotide and amino acid sequence of the URAT1(L)GFP construct is set out in FIG. 1B, which also shows alignment of the nucleotide sequence with NM_{—}144585.

Lentiviral particles were generated according to ViraPower HiPerform expression system procedure (Invitrogen Life Technologies) and used to transduce CHO cells. Blasticidin selection enabled the generation of a stable clonal pool of cells, confirmed by expression of GFP and V5 epitope. The clonal pools were sorted using fluorescence-activated cell sorting (FACS) on the basis of GFP expression with the gating set at the top 50% of expression into single cells which were subsequently expanded to generate clonal lines. One clone was identified with the best assay performance as determined by maximal separation between complete inhibition of uric acid transport (with 10 μM benzbromarone) and no inhibition (DMSO). This cell line was used for all screening activities and is referred to as CHO-URAT1(L)GFP#8 or CHO#8.

### 2. URAT-1 Inhibitor Activity

The potency of the compounds of formula (I) as inhibitors of the URAT-1 transporter was determined as follows.

CHO#8 cells were cultured in cell line maintenance flasks in medium consisting of Dulbecco's modified Eagle medium (DMEM) with high glucose and sodium pyruvate (4.5 g of glucose per litre, Invitrogen Life Technologies), supplemented with heat-inactivated foetal bovine serum (FBC, 10% v/v), 1×NEAA (non-essential amino acids) and blasticidin (10 μg/ml). Cultures were grown in 175 cm² tissue culture flasks in a humidified incubator at approximately 37° C. in approximately 95% air/5% CO₂. Near confluent CHO#8 cell cultures were harvested by trypsinisation, re-suspended in culture medium and the process was repeated once or twice weekly to provide sufficient cells for use.

Assay ready flasks were generated by the same method, except the cells were not cultured in blasticidin.

Assay ready frozen cells were generated by freezing 40,000,000 cells in 1 ml of FBS (without blasticidin) containing 10% DMSO per vial. One vial was sufficient for 5 assay plates. Each vial was thawed rapidly to 37° C., washed and re-suspended in pre-warmed culture medium for seeding onto assay plates.

CHO#8 cells were seeded onto Cytostar™ 96-well plates at a density of 5×10⁵ cells per well. The cells were cultured for 1 day at approximately 37° C. in a humidified incubator containing approximately 5% CO₂ in air. After approximately 24 hours culture, cells were used for uptake experiments.

On the day of assay, culture medium was removed from the wells and the cells were washed once with 50 μL of chloride-containing buffer (136.7 mM NaCl, 5.36 mM KCl, 0.952 mM CaCl₂, 0.441 mM KH₂PO₄, 0.812 mM MgSO₄, 5.6 mM D-glucose, 0.383 mM Na₂HPO₄.2H₂O, 10 mM HEPES, pH 7.4 with NaOH). The cells were pre-incubated with another 50 μL of chloride-containing buffer for one hour at approximately 37° C. in a humidified incubator containing approximately 5% CO₂ in air.

Assay compound plates were prepared by diluting the compounds of formula (I) with chloride-free buffer (125 mM Na-gluconate, 4.8 mM K-gluconate, 1.3 mM Ca-gluconate, 1.2 mM KH₂PO₄, 1.2 mM MgSO₄, 5.6 mM D-glucose, 25 mM HEPES, pH 7.4 with NaOH) in 100% DMSO to a final concentration of 1% DMSO. [¹⁴C]-Uric Uric acid working stock was made by addition of radiolabeled compound to a final concentration of 120 nM in chloride-free buffer. In all wells, the final assay concentration of solvent (DMSO) was 0.25%; the final assay concentration of [¹⁴C]-uric acid was 30 nM in chloride-free buffer and the final compound of formula (I) concentrations ranged from 0 to 10 μM. The vehicle comparator was DMSO (i.e. no inhibition of uric acid transport) and the pharmacological blockade (i.e. 100% inhibition of uric acid transport) was defined by benzbromarone at 10 μM final assay concentration.

After pre-incubation, cells were washed with 50 μL of chloride-free buffer and another 50 μL of chloride-free buffer was added. Thereafter, 25 μL of compound of formula (I) was added from the prepared compound plate and the cells were pre-incubated for 15 minutes prior to the addition of 25 mL of [¹⁴C] uric acid. The plate was incubated at room temperature and protected from light for three hours prior to measuring proximity-induced scintillation on a Wallac microbeta at 1 minute/well.

The accumulation of [¹⁴ C]-uric acid into CHO#8 cells was calculated and the ₁₀₅₀ (μM) values, defined as the concentration of inhibitor required for 50% inhibition of transport, were determined from a 4 parameter logistic fit to generate sigmoid curves from dose response data.

### IC₅₀ (μM) Values: Exs 1-332

| Ex. | IC₅₀ |
|---|---|
| 1 | 0.058 |
| 2 | 0.064 |
| 3 | 0.069 |
| 4 | 3.358 |
| 5 | >10.000 |
| 6 | >10.000 |
| 7 | 0.141 |
| 8 | 2.142 |
| 9 | >2.805 |
| 10 | >5.759 |
| 11 | 1.384 |
| 12 | 0.354 |
| 13 | 1.266 |
| 14 | >2.899 |
| 15 | >7.370 |
| 16 | 0.428 |
| 17 | 0.161 |
| 18 | >10.000 |
| 19 | >10.000 |
| 20 | >7.523 |
| 21 | >10.000 |
| 22 | 3.159 |
| 23 | >10.000 |
| 24 | >10.000 |
| 25 | >10.000 |
| 26 | 0.875 |
| 27 | >10.000 |
| 28 | >7.448 |
| 29 | >6.081 |
| 30 | 0.609 |
| 31 | 2.222 |
| 32 | 0.986 |
| 33 | 6.187 |
| 34 | NT |
| 35 | 4.325 |
| 36 | 1.945 |
| 37 | >5.655 |
| 38 | >7.103 |
| 39 | 0.994 |
| 40 | >2.918 |
| 41 | 1.350 |
| 42 | >3.087 |
| 43 | 0.877 |
| 44 | 4.331 |
| 45 | 1.020 |
| 46 | 0.504 |
| 47 | 5.389 |
| 48 | 1.965 |
| 49 | 1.459 |
| 50 | 2.548 |
| 51 | 2.137 |
| 52 | 0.650 |
| 53 | 5.239 |
| 54 | 2.854 |
| 55 | 0.590 |
| 56 | 2.119 |
| 57 | 0.517 |
| 58 | >10.000 |
| 59 | >10.000 |
| 60 | >8.784 |
| 61 | >10.000 |
| 62 | 0.722 |
| 63 | 1.438 |
| 64 | 8.006 |
| 65 | 1.487 |
| 66 | 5.812 |
| 67 | >7.577 |
| 68 | >8.469 |
| 69 | >6.343 |
| 70 | >10.000 |
| 71 | >10.000 |
| 72 | >10.000 |
| 73 | >10.000 |
| 74 | >3.445 |
| 75 | >10.000 |
| 76 | 0.093 |
| 77 | 0.160 |
| 78 | 0.165 |
| 79 | >10.000 |
| 80 | >5.780 |
| 81 | 3.605 |
| 82 | 0.340 |
| 83 | 1.138 |
| 84 | 0.681 |
| 85 | 0.845 |
| 86 | 0.680 |
| 87 | 0.415 |
| 88 | >6.718 |
| 89 | 2.826 |
| 90 | 1.667 |
| 91 | 2.628 |
| 92 | >7.861 |
| 93 | >1.614 |
| 94 | 0.621 |
| 95 | >7.363 |
| 96 | 0.675 |
| 97 | 1.174 |
| 98 | >1.688 |
| 99 | 1.632 |
| 100 | 0.554 |
| 101 | 1.760 |
| 102 | 1.426 |
| 103 | >6.784 |
| 104 | 2.620 |
| 105 | >1.787 |
| 106 | >1.340 |
| 107 | >6.781 |
| 108 | 0.345 |
| 109 | 0.552 |
| 110 | >7.537 |
| 111 | 0.871 |
| 112 | 4.028 |
| 113 | 1.124 |
| 114 | 4.411 |
| 115 | 1.626 |
| 116 | >5.310 |
| 117 | 1.443 |
| 118 | 1.377 |
| 119 | 1.575 |
| 120 | 1.323 |
| 121 | 1.039 |
| 122 | 0.581 |
| 123 | 1.246 |
| 124 | >6.362 |
| 125 | 0.551 |
| 126 | 0.699 |
| 127 | 2.294 |
| 128 | 1.194 |
| 129 | 0.480 |
| 130 | 0.414 |
| 131 | 0.297 |
| 132 | 2.595 |
| 133 | 1.583 |
| 134 | 0.718 |
| 135 | 1.728 |
| 136 | 0.464 |
| 137 | >8.947 |
| 138 | 3.466 |
| 139 | 3.384 |
| 140 | >7.302 |
| 141 | 0.737 |
| 142 | 0.540 |
| 143 | 0.566 |
| 144 | NT |
| 145 | 0.803 |
| 146 | >10.000 |
| 147 | >10.000 |
| 148 | 0.118 |
| 149 | 0.494 |
| 150 | 2.509 |
| 151 | 0.508 |
| 152 | >10.000 |
| 153 | NT |
| 154 | 1.629 |
| 155 | >7.249 |
| 156 | 3.431 |
| 157 | 2.882 |
| 158 | 3.211 |
| 159 | 0.499 |
| 160 | 0.347 |
| 161 | >4.853 |
| 162 | 0.565 |
| 163 | 0.681 |
| 164 | >10.000 |
| 165 | 0.505 |
| 166 | 2.679 |
| 167 | 0.166 |
| 168 | 0.161 |
| 169 | 0.073 |
| 170 | >1.263 |
| 171 | 0.671 |
| 172 | 0.917 |
| 173 | >3.658 |
| 174 | 0.237 |
| 175 | >5.439 |
| 176 | >5.996 |
| 177 | 0.584 |
| 178 | 0.645 |
| 179 | 0.121 |
| 180 | 0.814 |
| 181 | 1.221 |
| 182 | 1.332 |
| 183 | >6.030 |
| 184 | >6.241 |
| 185 | NT |
| 186 | 1.081 |
| 187 | >6.638 |
| 188 | 0.688 |
| 189 | >10.000 |
| 190 | >10.000 |
| 191 | >10.000 |
| 192 | >10.000 |
| 193 | 0.834 |
| 194 | >7.151 |
| 195 | 2.244 |
| 196 | 1.577 |
| 197 | 0.225 |
| 198 | 2.192 |
| 199 | >6.369 |
| 200 | >10.000 |
| 201 | 0.651 |
| 202 | 0.823 |
| 203 | 0.387 |
| 204 | 0.317 |
| 205 | >6.812 |
| 206 | >3.316 |
| 207 | 0.747 |
| 208 | 1.786 |
| 209 | >7.250 |
| 210 | 1.605 |
| 211 | 1.112 |
| 212 | 0.782 |
| 213 | 2.044 |
| 214 | >10.000 |
| 215 | >10.000 |
| 216 | 1.420 |
| 217 | >10.000 |
| 218 | >1.665 |
| 219 | 0.426 |
| 220 | 0.110 |
| 221 | >10.000 |
| 222 | >3.127 |
| 223 | >10.000 |
| 224 | >10.000 |
| 225 | 0.300 |
| 226 | >10.000 |
| 227 | >10.000 |
| 228 | 0.123 |
| 229 | >10.000 |
| 230 | 0.747 |
| 231 | >10.000 |
| 232 | 0.513 |
| 233 | >10.000 |
| 234 | >10.000 |
| 235 | >10.000 |
| 236 | >10.000 |
| 237 | >10.000 |
| 238 | >10.000 |
| 239 | >10.000 |
| 240 | 0.577 |
| 241 | >10.000 |
| 242 | 0.967 |
| 243 | 0.077 |
| 244 | 0.365 |
| 245 | 1.996 |
| 246 | 0.699 |
| 247 | 0.450 |
| 248 | >1.203 |
| 249 | >10.000 |
| 250 | 1.884 |
| 251 | 1.322 |
| 252 | 1.111 |
| 253 | 2.133 |
| 254 | 0.878 |
| 255 | 0.684 |
| 256 | >4.820 |
| 257 | 1.202 |
| 258 | 3.115 |
| 259 | 3.135 |
| 260 | 1.964 |
| 261 | 1.822 |
| 262 | 3.585 |
| 263 | 2.250 |
| 264 | 1.650 |
| 265 | 2.428 |
| 266 | 1.567 |
| 267 | 0.930 |
| 268 | 1.473 |
| 269 | 0.278 |
| 270 | 0.957 |
| 271 | 4.538 |
| 272 | >4.317 |
| 273 | >10.000 |
| 274 | >10.000 |
| 275 | >10.000 |
| 276 | >10.000 |
| 277 | >5.433 |
| 278 | 2.737 |
| 279 | >10.000 |
| 280 | 2.292 |
| 281 | 3.254 |
| 282 | 1.664 |
| 283 | >8.573 |
| 284 | 2.525 |
| 285 | 5.589 |
| 286 | 1.006 |
| 287 | 1.436 |
| 288 | 0.431 |
| 289 | 0.479 |
| 290 | 1.095 |
| 291 | 1.131 |
| 292 | 1.130 |
| 293 | >6.146 |
| 294 | 0.888 |
| 295 | 0.478 |
| 296 | >10.000 |
| 297 | 1.007 |
| 298 | 4.388 |
| 299 | 4.368 |
| 300 | 0.329 |
| 301 | 0.382 |
| 302 | 1.065 |
| 303 | >1.427 |
| 304 | 1.013 |
| 305 | >5.780 |
| 306 | 2.413 |
| 307 | >7.187 |
| 308 | >10.000 |
| 309 | >10.000 |
| 310 | 0.864 |
| 311 | 1.096 |
| 312 | >6.453 |
| 313 | 3.170 |
| 314 | >10.000 |
| 315 | 2.185 |
| 316 | >10.000 |
| 317 | 1.635 |
| 318 | 0.145 |
| 319 | >1.714 |
| 320 | >7.607 |
| 321 | 0.275 |
| 322 | 2.991 |
| 323 | 2.171 |
| 324 | 3.275 |
| 325 | 2.004 |
| 326 | 0.369 |
| 327 | 0.619 |
| 328 | >0.830 |
| 329 | 0.606 |
| 330 | 2.387 |
| 331 | >6.715 |
| 332 | >2.378 |

NT = Not Tested

### IC₅₀ (μM) Values: Exs 333-341

| Ex. | IC₅₀ |
|---|---|
| 333 | 7.537 |
| 334 | 2.029 |
| 335 | 3.259 |
| 336 | 3.910 |
| 337 | 0.112 |
| 338 | 0.797 |
| 339 | >10.000 |
| 340 | 0.484 |
| 341 | 0.009 |

1. A compound of formula (I): 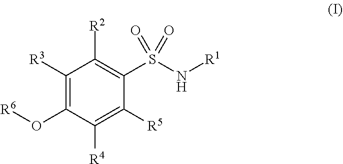
or a pharmaceutically acceptable salt thereof, wherein
R¹ is a ‘C-linked’ 5-membered heteroaryl containing one, two or three heteroatoms selected from: (a) one to three nitrogen atoms, (b) one or two nitrogen atoms and one sulphur atom or (c) one or two nitrogen atoms and one oxygen atom, wherein said heteroaryl is optionally substituted on a ring carbon atom by, valency permitting, one, two or three X¹;
each X¹ is independently selected from: F; Cl; CN; (C₁-C₄)alkyl optionally substituted by one, two or three F; or (C₁-C₄)alkyloxy optionally substituted by one two or three F;
R², R³ and R⁵ are independently selected from: H; halogen; CN; (C₁-C₄)alkyl optionally substituted by one, two or three F; or (C₁-C₄)alkyloxy optionally substituted by one, two or three F;
R⁴ is selected from: halogen; CN; (C₁-C₄)alkyl optionally substituted by one, two or three F; or (C₁-C₄)alkyloxy optionally substituted by one, two or three F;
R⁶ is phenyl substituted by one, two or three X²; or a ‘C-linked’ 6-membered heteroaryl containing one or two nitrogen atoms wherein said heteroaryl is optionally substituted by one, two or three X²;
each X² is independently selected from: F; Cl; CN; —S(C₁-C₄)alkyl; —NR⁷R⁸; (C₁-C₆)alkyloxy optionally substituted by one, two or three F; (C₃-C₆)cycloalkyloxy; (C₁-C₆)alkyl optionally substituted by one, two or three F; or (C₁-C₆)alkyl substituted by OH; and
R⁷ and R⁸ are independently H or (C₁-C₄)alkyl or, together with the nitrogen atom to which they are attached, form a saturated 4- to 6-membered nitrogen containing monocycle.

2. The compound according to claim 1 or a pharmaceutically acceptable salt thereof, wherein R¹ is a ‘C-linked’ 5-membered heteroaryl containing one or two nitrogen atoms and one sulphur atom, wherein said heteroaryl is optionally substituted by one or two X¹.

3. The compound according to claim 2 or a pharmaceutically acceptable salt thereof, wherein said heteroaryl is optionally substituted by one X¹.

4. The compound according to claim 1 or a pharmaceutically acceptable salt thereof, wherein R¹ is a ‘C-linked’ 5-membered heteroaryl containing one or two nitrogen atoms and one oxygen atom, wherein said heteroaryl is optionally substituted by one or two X¹.

5. The compound according to claim 4 or a pharmaceutically acceptable salt thereof, wherein R¹ is a ‘C-linked’ 5-membered heteroaryl containing one nitrogen atom and one oxygen atom, wherein said heteroaryl is substituted by one X¹.

6. The compound according to claim 1 or a pharmaceutically acceptable salt thereof, wherein each X¹ is independently selected from: F; Cl; or (C₁-C₄)alkyl optionally substituted by one, two or three F.

7. The compound according to claim 1 or a pharmaceutically acceptable salt thereof, wherein R², R³ and R⁵ are independently selected from: H; halogen; CN; (C₁-C₃)alkyl; or (C₁-C₃)alkyloxy; and R⁴ is selected from: halogen; CN; (C₁-C₃)alkyl; or (C₁-C₃)alkyloxy.

8. The compound according to claim 1 or a pharmaceutically acceptable salt thereof, wherein R² is H or halogen wherein said halogen is F; R³ is H; R⁴ is halogen wherein said halogen is Cl, Br, or I; CN or (C₁-C₃)alkyl; and R⁵ is H.

9. The compound according to claim 1 or a pharmaceutically acceptable salt thereof, wherein R⁶ is phenyl substituted by one, two or three X².

10. The compound according to claim 1 or a pharmaceutically acceptable salt thereof, wherein R⁶ is ‘C-linked’ pyridinyl substituted by one, two or three X².

11. The compound according to claim 1 or a pharmaceutically acceptable salt thereof, wherein R⁶ is ‘C-linked’ pyridinyl substituted by one X².

12. The compound according to claim 1 or a pharmaceutically acceptable salt thereof, wherein each X² is independently selected from: F; CI; CN; (C₁-C₄)alkyloxy optionally substituted by one, two or three F; (C₃-C₆)cycloalkyloxy; (C₁-C₄)alkyl optionally substituted by one, two or three F; or (C₁-C₄)alkyl substituted by OH.

13. A pharmaceutical composition comprising a compound according to claim 1 or a pharmaceutically acceptable salt thereof, and a pharmaceutically acceptable excipient.

14. The pharmaceutical composition according to claim 13, wherein the composition further comprises one or more additional therapeutic agents.

15. A method of treating a disorder in a human or animal for which a URAT-1 inhibitor is indicated, comprising administering to said human or animal a therapeutically effective amount of a compound according to claim 1, or a pharmaceutically acceptable salt thereof.

16. The method according to claim 15, wherein the disorder for which a URAT-1 inhibitor is indicated is gout.


SEQUENCE LISTING (3 sequences)

SEQ ID NO: 1 - DNA, 1692 bp, Homo sapiens
gggcccctcttctgggccccttgagtaggttccatggcattttctgaactcctggacctcgtgggtggcctgggcaggttccaggttctccagacgatggctctgatggtctccatcatgtggctgtgtacccagagcatgctggagaacttctcggccgccgtgcccagccaccgctgctgggcacccctcctggacaacagcacggctcaggccagcatcctagggagcttgagtcctgaggccctcctggctatttccatcccgccgggccccaaccagaggccccaccagtgccgccgcttccgccagccacagtggcagctcttggaccccaatgccacggccaccagctggagcgaggccgacacggagccgtgtgtggatggctgggtctatgaccgcagcatcttcacctccacaatcgtggccaagtggaacctcgtgtgtgactctcatgctctgaagcccatggcccagtccatctacctggctgggattctggtgggagctgctgcgtgcggccctgcctcagacaggtttgggcgcaggctggtgctaacctggagctaccttcagatggctgtgatgggtacggcagctgccttcgcccctgccttccccgtgtactgcctgttccgcttcctgttggcctttgccgtggcaggcgtcatgatgaacacgggcactctcctgatggagtggacggcggcacgggcccgacccttggtgatgaccttgaactctctgggcttcagcttcggccatggcctgacagctgcagtggcctacggtgtgcgggactggacactgctgcagctggtggtctcggtccccttcttcctctgctttttgtactcctggtggctggcagagtcggcacgatggctcctcaccacaggcaggctggattggggcctgcaggagctgtggagggtggctgccatcaacggaaagggggcagtgcaggacaccctgacccctgaggtcttgctttcagccatgcgggaggagctgagcatgggccagcctcctgccagcctgggcaccctgctccgcatgcccggactgcgcttccggacctgtatctccacgttgtgctggttcgcctttggcttcaccttcttcggcctggccctggacctgcaggccctgggcagcaacatcttcctgctccaaatgttcattggtgtcgtggacatcccagccaagatgggcgccctgctgctgctgagccacctgggccgccgccccacgctggccgcatccctgttgctggcagggctctgcattctggccaacacgctggtgccccacgaaatgggggctctgcgctcagccttggccgtgctggggctgggcggggtgggggctgccttcacctgcatcaccatctacagcagcgagctcttccccactgtgctcaggatgacggcagtgggcttgggccagatggcagcccgtggaggagccatcctggggcctctggtccggctgctgggtgtccatggcccctggctgcccttgctggtgtatgggacggtgccagtgctgagtggcctggccgcactgcttctgcccgagacccagagcttgccgctgcccgacaccatccaagatgtgcagaaccaggcagtaaagaaggcaacacatggcacgctggggaactctgtcctaaaatccacacagttt

SEQ ID NO: 2 - DNA, 2546 bp, Artificial Sequence
  Primer
acaagtttgtacaaaaaagaggcttcgccaccatggccttcagcgagctgctggacctggtgggaggcctgggcagattccaggtgctgcagaccatggccctgatggtgtccatcatgtggctgtgcacccagagcatgctggaaaacttctctgccgccgtgcccagccacagatgctgggcccctctgctggacaacagcaccgcccaggccagcatcctgggcagcctgtctccagaggccctgctggccatcagcatcccccctggccccaaccagaggccccaccagtgcagacggttccggcagcctcagtggcagctgctggatcccaacgccaccgccacctcttggagcgaggccgacaccgagccctgtgtggacggctgggtgtacgaccggtccatcttcaccagcaccatcgtggccaagtggaacctggtgtgcgacagtcacgccctgaagcccatggcccagagcatctacctggccggcattctggtgggagccgccgcttgtggccctgccagcgatagattcggcagacggctggtgctgacctggtcctacctgcagatggccgtgatgggcaccgccgcagcctttgcccctgccttccctgtgtactgcctgttccggttcctgctggccttcgccgtggccggcgtgatgatgaacaccggcaccctgctgatggaatggaccgccgccagagccagacccctggtgatgaccctgaacagcctgggcttcagcttcggacatggcctcacagccgctgtggcttatggcgtgcgggactggacactgctgcagctggtggtgtccgtgcccttcttcctgtgcttcctgtacagctggtggctcgctgagagcgcccggtggctgctgaccacaggcagactggactggggcctgcaggaactgtggcgggtcgccgccatcaatggcaagggcgccgtgcaggacaccctgacccctgaggtgctgctgagcgccatgcgcgaggaactgagcatgggccagcctccagccagcctgggcacactgctgagaatgcccggcctgcggttccggacctgcatcagcaccctgtgttggttcgccttcggcttcaccttcttcggcctggccctggacctccaggccctgggcagcaacatcttcctgctgcagatgttcatcggcgtggtggacatccccgccaagatgggcgccctgctgctgctgtctcacctgggcagaaggcctaccctggccgcctctctgctgctggccggactgtgcatcctggccaacaccctggtgccccacgagatgggagccctgagatctgccctggccgtcctgggactgggaggcgtgggagctgccttcacctgtatcaccatctacagcagcgagctgttccccaccgtgctgcggatgacagccgtgggcctgggacagatggccgccagaggcggagccatcctgggacctctggtgcgcctgctgggagtgcacggaccttggctccctctgctggtgtacggcaccgtgcctgtgctgtctggactggctgctctgctgctgcccgagacacagagcctgcccctgcccgacaccatccaggacgtgcagaaccaggccgtgaagaaggccacccacggcaccctgggcaacagcgtgctgaagtccacccagttcatggtgtccaagggggaggaactgtttaccggcgtggtgcccatcctggtggaactggacggcgacgtgaacggccacaagttcagcgtgtccggcgagggcgaaggcgacgccacctacggcaagctgaccctgaagttcatctgcaccaccggcaagctgcccgtgccttggcccaccctggtgacaaccttcacctacggcgtgcagtgcttcgccagataccccgaccacatgaagcagcacgatttcttcaagtccgccatgcccgagggctacgtgcaggaacggaccatcttcttcaaggacgacggcaactacaagaccagagccgaagtgaagttcgagggcgataccctggtgaaccggatcgagctgaagggcatcgacttcaaagaggacggcaatatcctgggccacaagctggagtacaactacaacagccacaaggtgtacatcaccgccgacaagcagaaaaacggcatcaaagtgaacttcaagacccggcacaacatcgaggacggaagcgtgcagctggccgaccactaccagcagaacacccccatcggcgacggccccgtgctgctgcctgacaaccactacctgagcacccagtccgccctgagcaaggaccccaacgagaagcgggaccacatggtgctgctggaattcgtgaccgccgctggcatcacactgggcatggacgagctgtacaagtacccagctttcttgtacaaagtggttgatatccagcacagtggcggccgctcgagtctagagggcccgcggttcgaaggtaagcctatccctaaccctctcctcggtctcgattctacgcgtaccggttagtaatga

SEQ ID NO: 3 - protein, 835 aa, Homo sapiens [sequence omitted]